SEQUENCE LISTING (1 sequence)

SEQ ID NO: 1 - protein, 291 aa, Thermomyces lanuginosus
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

(19) United States
(12) Patent Application Publication
(10) Pub. No.: US 2025/0361534 A1
(43) Pub. Date: Nov. 27, 2025

(54) BATCH PROCESS FOR ENZYMATIC MODIFICATION OF LIPIDS

(71) Applicants: Bunge SA, Geneva (CH); EnginZyme AB, Solna (SE)

(72) Inventor: Christopher Loren Gene Dayton, Jupiter, FL (US)

(21) Appl. No.: 18/869,649

(22) PCT Filed: May 29, 2023
(86) PCT No.: PCT/SE2023/050519

§ 371 Date: Nov. 26, 2024

Related U.S. Application Data
(60) Provisional application No. 63/346,489, filed on May 27, 2022.

(51) Int. Cl. C12P 7/6458; C12N 1/14; C12N 9/20; C12N 11/14

(52) U.S. Cl. CPC C12P 7/6458; C12N 1/14; C12N 9/20; C12N 11/14; C12Y 301/01003

(57) ABSTRACT

The present disclosure is directed to a batch process for the enzymatic treatment of lipid-containing compositions, and more particularly, to a batch process for the manufacture of interesterified fats using enzymes. Advantageously, the process of the present disclosure can be performed using low amounts of enzymes, and shorter time frames than typical batch enzymatic interesterification processes. The claimed process can be used to replace batch chemical interesterification processes

### CROSS-REFERENCE TO RELATED APPLICATIONS

This application claims priority to U.S. Provisional Application No. 63/346,489, filed on May 27, 2022, which is herein incorporated by reference in its entirety.

### SEQUENCE LISTING

The instant application contains a Sequence Listing which has been submitted electronically in XML format and is hereby incorporated by reference in its entirety. Said XML copy, created on May 25, 2023, is named ‘35893-616SEQ’ and is 2 kilobytes in size.

### BACKGROUND OF THE INVENTION

The present disclosure is directed to a batch process for the enzymatic treatment of lipid-containing compositions, and more particularly, to a batch process for the manufacture of interesterified fats using enzymes. Advantageously, the process of the present disclosure can be performed using low amounts of enzymes, and shorter time frames than typical batch enzymatic interesterification processes. The claimed process can be used to replace batch chemical interesterification processes.

Fats are made of fatty acids attached to a three-carbon glycerol backbone. Fatty acids are made up of chains of carbon atoms with a terminal hydroxyl group. The hydroxyl groups can attach to one, two, or three of the hydroxyl groups on the glycerol backbone to form mono-, di-, or triacylglycerols, or fats. The functional and nutritional qualities of the fats will depend on a variety of factors including whether they are monoacylglycerol (MAG), a diacylglycerol (DAG) or a tri-acylglycerol (TAG); the number of carbons in the fatty acid chains; whether the chains are saturated, mono-unsaturated, or poly-unsaturated; whether any unsaturated double bonds in the chains are in the form of the cis or trans isomer; the location of any double bonds along the chains; and the positions of the different types of fatty acids relative to the three carbons of the glycerol backbone.

Lipids are a classification of a broad variety of chemical substances characterized as fats, oils, waxes, and phospholipids. Included within this broad classification are triglycerides, diglycerides, monoglycerides, fatty acids, fatty alcohols, soaps and detergents, terpenes, steroids, and vitamins A, E, D2, and K2. Lipids can be obtained from oilseeds such as soybeans, canola, rapeseed, sunflower, palm, and olives; animal products such as fish, pork, and beef, and synthetic compounds or synthetically derived compositions such as structured lipids for nutritional applications, oleochemicals for industrial and pharmaceutical applications, and biodiesel for energy. Vegetable oils are obtained by pressing or solvent extraction of the oil from the oilseed. The crude oils contain many minor components. Some of these components are detrimental to the performance or aesthetic properties of the oils; others, such as sterols and tocopherols, are nutritionally beneficial.

In recent years there has been increased interest in providing alternatives to the high trans fats and shortening products used in traditional food preparation. Traditionally, liquid oils were manufactured into functional fats containing solids for various margarine and shortening products by nickel hydrogenation. Such hydrogenation processes led to the formation of trans fatty acids. It is believed that fats having reduced trans fatty acids may provide certain health benefits to the consumer. Accordingly, many large food producers are replacing high trans fats with low or even zero trans fats compositions. Originally, efforts at providing low trans fats products focused on reducing the level of hydrogenation of the fat products. More recently, efforts have focused on changing the structure of a liquid oil to change the melting properties and functionality without changing the fatty acid composition or generating trans fatty acids. One method of achieving this is a process known as interesterification.

Interesterification is a known reaction of triacylglycerol structures whereby individual fatty acid structures at positions of the triglyceride being interesterified are interchanged on the glycerol moiety. This is at times referred to or recognized as a randomization wherein fatty acid moieties from one glycerol component of a triacylglycerol are exchanged with those of a glycerol component of another triacylglycerol. This results in triacylglycerol structures which have interchanged fatty acid moieties that vary from glycerol structure to glycerol structure.

The art of interesterification has developed to enable the production of, for example, triglyceride compositions which provide certain melt profiles that can be of interest in certain applications. Generally these are recognized herein as “structured lipids” to distinguish the interesterified products from physical blends of the same components that have not been subjected to interesterification. Swern, Bailey's Industrial Oil and Fat Products, 3rd edition, pages 941-970 (1964) described the reesterification of fatty acids and glycerol, mono- and poly-hydroxy alcohols, interesterification (acidolysis and alcoholysis), and transesterification of lipids via chemical methods.

Interesterification can be accomplished either chemically or enzymatically. Chemical interesterification (CIE) has been the industry standard for many years, and allows the most flexible product mix and various batch sizes. Chemical interesterification is generally accomplished with a chemical such as sodium methoxide (NaOCH₃). The CIE reaction is fast, with the reaction usually taking around 30 minutes, with a batch cycle time of 4 to 8 hours, including loading/drying, chemical dosing and dispersion, verification of reaction endpoint, reaction inactivation with water or acid solution, and discharge. The reacted oil/fat is moved to a bleacher, where most of the unwanted reaction byproducts are removed through silica and/or bleaching clay, and then filtered.

While chemical interesterification is rapid and can be less costly than enzymatic interesterification in terms of the chemical in comparison with the enzyme, it has several distinct disadvantages. The sodium methoxide chemical is very caustic and reactive with water, so it is dangerous and difficult to handle. The CIE process can also generate large amounts of by-products, such as sodium soap, fatty acid methyl esters (FAME), partial glycerides, and process contaminants, and thus requires further processing steps to remove the unwanted by-products (e.g., water washing to remove the bulk of the by-products; use of adsorbents to remove unwanted color compounds generated during the CIE process, etc.). Additionally, if water washing is used for the neutralization process, then CIE will see an increased neutral oil loss due to emulsion in the separation step. If acid is used, the additional loss will be seen in the removal of the Free Fatty Acids (FFA) in the physical refining process (deodorization). Overall, the basic yield loss calculation for CIE is 10 times the amount of sodium methoxide added. CIE can thus result in relatively high oil losses, and require the use of additional processing steps to remove contaminants and by-products.

The industry has been moving towards the enzymatic interesterification (EIE) process because it is selective for the hydrolysis of fatty acids (the interchange of the fatty acids along the glycerol backbone) and does not generate the losses or require the interesterified material to be re-bleached or otherwise processed to remove contaminants and by-products. Enzymes can also afford a great deal of control over the structure of the final interesterified product. In particular the use of certain enzymes can result in interesterification specifically at the 1- and 3-positions along the glycerol backbone chain, exactly where it is most desired. While enzymatic catalysts were originally used only for high value-added products, they are now being used increasingly in the manufacture of commodity fats and fat blends.

One particularly preferred enzyme catalyst is the lipase from Thermomyces lanuginosus. This enzyme is specific for the 1 and 3 sites on the glycerol backbone, and it is heat stable up to about 75° C. This enzyme, however, can be readily inactivated by radicals such as peroxides, certain polar impurities such as phosphatides and soaps, secondary oxidation products such as ketones and aldehydes, and trace metals. Thus, the quality of the oil feedstock is important.

An immobilized granulated form of the lipase from Thermomyces lanuginosus is sold by Novozymes Corporation under the registered trademark Lipozyme® TL IM. The product literature that comes with this enzyme product discloses a process of use comprising cooling the lipids to 70° C., pumping the lipids to a single reactor column or tank, and passing the oil through the column or mixing the oil with the enzyme in the tank. The lipids contact the enzyme in the column or tank and are continuously interesterified. The interesterified lipids may then be blended with other lipids, or deodorized, or shipped to the final customer.

While EIE may have certain economic advantages in comparison with CIE, part of the economic advantages relates to the continuous production of single base stocks in large pipe-through reactors. Such a design, however, brings more difficult changeover and commingling, and the packed bed limits the flexibility somewhat and is best utilized with large commodity type bases. Additionally, continuous EIE processes require large catalyst (enzyme) in process usage of roughly 1 kg of oil/kg of immobilized enzyme with a contact time of at least 1 hour. The overall usage rate through the life of the enzyme in a continuous packed bed reactor system as described in U.S. Pat. No. 8,361,763 is 0.4 kg of enzyme per metric tonne of oil treated. Current batch EIE processes also require large enzyme process usage (e.g., around 5% w/w), and long reaction times (typically around 24 hours).

It is thus an object of the present disclosure to provide an enzymatic interesterification process that can replace the traditional CIE process, and which thus eliminates formation of the process contaminates present following CIE treatment. It is a further object of the present disclosure to provide an EIE process that does not require large amounts of in process enzyme catalyst, and which has shorter reaction times and greater flexibility than continuous EIE processes.

### SUMMARY OF THE DISCLOSURE

The present disclosure is directed to a batch process for the enzymatic treatment of lipid-containing compositions, and more particularly, to a batch process for the manufacture of interesterified fats using enzymes. Advantageously, the process of the present disclosure can be performed using low amounts of enzymes, and shorter time frames than typical batch enzymatic interesterification processes. The claimed process can replace batch chemical interesterification processes.

In one aspect, the disclosure is directed to a process for batch enzymatic treatment of a lipid-containing composition, the process comprising: providing the lipid-containing composition; contacting the lipid-containing composition with an immobilized enzyme material comprising a carrier and at least one enzyme immobilized on the carrier, wherein the amount of the immobilized enzyme material is about 1% or less (by weight of the lipid-containing composition); and mixing the lipid-containing composition and the immobilized enzyme material to form an enzymatically treated composition.

In another aspect, the disclosure is directed to a process for batch enzymatic treatment of multiple lipid-containing compositions, the process comprising: providing a lipid-containing composition; contacting the lipid-containing composition with an immobilized enzyme material comprising a carrier and at least one enzyme immobilized on the carrier, wherein the amount of the immobilized enzyme material is about 0.72% or less (by weight of the lipid-containing composition); mixing the lipid-containing composition with the immobilized enzyme material to form a first enzymatically treated composition, wherein the mixing occurs for about 10 hours or less; separating the immobilized enzyme material from the first enzymatically treated composition; contacting the immobilized enzyme material with at least one additional lipid-containing composition; and mixing the at least one additional lipid-containing composition with the immobilized enzyme material to form a second enzymatically treated composition.

### DETAILED DESCRIPTION OF THE INVENTION

The present disclosure is directed to a batch process for the enzymatic treatment of lipid-containing compositions, and more particularly, to a batch process for the manufacture of interesterified fats using enzymes. Advantageously, the process of the present disclosure can be performed using low amounts of enzymes, and shorter time frames than typical batch enzymatic interesterification processes. The claimed process can be used to replace batch chemical interesterification processes.

Thus, in one aspect, the present disclosure is directed to a process for batch enzymatic treatment of a lipid-containing composition. The process comprises providing the lipid-containing composition; contacting the lipid-containing composition with an immobilized enzyme material comprising a carrier and at least one enzyme immobilized on the carrier, wherein the amount of the immobilized enzyme material is about 1% or less, or about 0.72% or less (by weight of the lipid-containing composition); and mixing the lipid-containing composition and the immobilized enzyme material to form an enzymatically treated composition. In certain embodiments, the process may further comprise separating the immobilized enzyme material from the enzymatically treated composition.

The enzymatic treatments described herein may be selected from the group consisting of interesterification, intraesterification, alcoholysis, acidolysis, glycerolysis, transesterification, and combinations thereof. In one particular embodiment, the enzymatic treatment is interesterification.

Advantageously, use of the immobilized enzyme materials described herein allow batch enzymatic interesterification to be performed using lower amounts of enzymes, and in a shorter time frame, than previously known EIE processes. Additionally, unlike traditional CIE processes, the EIE processes of the present disclosure do not result in formation of contaminants, and thus do not require additional processing of the compositions to remove process contaminants and/or by-products (e.g., water washing), or the use of adsorbents (e.g., silica, activated bleaching clay, activated carbon) for removal of unwanted color compounds generated in CIE.

It has further been discovered that the immobilized enzyme materials described herein retain their ability to catalyze EIE reactions, even after multiple uses. Without wishing to be bound to any particular theory, it is believed that the bonding of the enzyme to the carrier allows for the enzyme to be loaded at a much higher level than other currently available immobilized enzymes. Additionally, it is believed that the orientation of the enzyme on the carrier reduces enzyme inactivation due to poisons (i.e., substances that block, interfere with the binding sites, or change the active site, and/or increase the thermal stability of the enzyme within the lipid matrix). The immobilized enzyme material of the present disclosure thus provides an unexpected advantage over other immobilized enzymes known for use in EIE processes.

Thus, in another aspect, the present disclosure is directed to a process for batch enzymatic treatment of multiple lipid-containing compositions. The process comprises providing a lipid-containing composition; contacting the lipid-containing composition with an immobilized enzyme material comprising a carrier and at least one enzyme immobilized on the carrier, wherein the amount of the immobilized enzyme material is about 1% or less, or about 0.72% or less (by weight of the lipid-containing composition); mixing the lipid-containing composition with the immobilized enzyme material to form a first enzymatically treated composition; separating the immobilized enzyme material from the first enzymatically treated composition; contacting the immobilized enzyme material with at least one additional lipid-containing composition; and mixing at least one additional lipid-containing composition with the immobilized enzyme material to form a second enzymatically treated composition. In one aspect, the lipid-containing composition and/or the at least one additional lipid-containing composition is mixed with the immobilized enzyme material for about 10 hours or less. In certain embodiments, the process may further comprise separating the immobilized enzyme material from the second enzymatically treated composition, and optionally repeating the process one or more additional times by contacting the immobilized enzyme material with one or more additional lipid-containing compositions in the amounts and under the conditions set forth herein.

As discussed herein, the processes of the present disclosure can be performed using lower amounts of enzyme than is typically required for EIE reactions. In general, the processes of the present disclosure comprise contacting the lipid-containing composition with an immobilized enzyme material of the present disclosure in an amount effective to catalyze the interesterification reaction. In a non-limiting embodiment, the immobilized enzyme material of the present disclosure is used in an amount of about 1 wt. % or less (by weight of the lipid-containing composition). In other embodiments, the amount of the immobilized enzyme material is about 0.72 wt % or less (by weight of the lipid-containing composition). In other embodiments, the amount of the immobilized enzyme material is from about 0.15 wt. % to about 1.0 wt. %, or from about 0.15 wt. % to about 0.72 wt. %, or from about 0.18 wt. % to about 0.72 wt. %, or from about 0.18 wt. % to about 0.5 wt. %, or from about 0.15 wt. % to about 0.48 wt. %, or from about 0.15 wt. % to about 0.4 wt. %, or from about 0.18 wt. % to about 0.36 wt. % (by weight of the lipid-containing composition). Unless otherwise indicated, the amounts of immobilized enzyme material given herein include the total amount of carrier and enzyme present in the material.

As discussed herein, in some embodiments, the immobilized enzyme materials described herein may be used in more than one enzymatic treatment, including in two, three, four, or more enzymatic treatments (referred to herein as a “used” or “reused” immobilized enzyme material). Thus, in some embodiments, 100% of the immobilized enzyme material used in the enzymatic process may be a used immobilized enzyme material. In other embodiments, the immobilized enzyme material used in the process of the present disclosure may be a mixture of a used and previously unused (also referred to herein as “fresh”) immobilized enzyme material. The total amount of used or used plus fresh immobilized enzyme material used in the processes of the present disclosure is about 1 wt. % or less, including about 0.72 wt % or less, or from about 0.15 wt. % to about 1.0 wt. %, or from about 0.15 wt. % to about 0.72 wt %, or from about 0.18 wt. % to about 0.72 wt. %, or from about 0.18 wt. % to about 0.5 wt. %, or from about 0.15 wt. % to about 0.48 wt. %, or from about 0.15 wt. % to about 0.4 wt. %, or from about 0.18 wt. % to about 0.36 wt. % (by weight of the lipid-containing composition). In embodiments where a mixture of used and fresh immobilized enzyme material is used, the fresh immobilized enzyme material is used in an amount of from about 10% to about 75% based on the weight of the used immobilized enzyme material.

In embodiments where the immobilized enzyme material is reused, the total amount of lipid-containing composition treated by the immobilized enzymatic material is more than the amount of lipid-containing composition treated in a single batch enzymatic treatment. Thus, the effective dosage of the immobilized enzyme material may be less than the dosage of immobilized enzyme material included in a single batch treatment. As used herein, “effective dosage” refers to the amount of immobilized enzyme material used by total weight of the lipid-containing composition treated across batches. In certain embodiments, the effective dosage of the immobilized enzyme material used in the processes of the present disclosure is about 1 wt. % or less, including about 0.72 wt % or less, from about 0.15 wt. % to about 1.0 wt. %, from about 0.15 wt. % to about 0.72 wt %, from about 0.18 wt. % to about 0.72 wt. %, from about 0.18 wt. % to about 0.5 wt. %, or from about 0.15 wt. % to about 0.48 wt. %, or from about 0.15 wt. % to about 0.4 wt. %, or from about 0.18 wt. % to about 0.36 wt. %.

In certain embodiments, the amount of enzyme present in the immobilized enzyme material (referred to herein as “enzyme loading”) is from about 3 to about 20% (by weight of the immobilized enzyme material).

The lipid-containing composition may be contacted with an immobilized enzyme material of the present disclosure at a temperature of from about 60° C. to about 100° C., including at a temperature of from about 65° C. to about 95° C., from about 70° C. to about 95° C., or from about 70° C. to about 90° C. In some embodiments, the lipid-containing composition may be contacted with an immobilized enzyme material of the present disclosure under atmospheric pressure. Following treatment, the immobilized enzyme material may be separated from the enzymatically treated composition using any suitable technique, including filtration.

In certain embodiments, the processes provided herein involve charging the lipid-containing composition and immobilized enzyme material into a reactor vessel. The reactor vessel may be any industrial reactor type. The reactor vessel may be a vessel specifically designed for batch enzymatic interesterification, a hydrogenation reactor, and/or a chemical interesterification reactor, such as a stirred-tank reactor operating in batch mode. In certain embodiments, such vessels have means for heating and/or cooling the composition during agitation. In certain embodiments, the lipid-containing composition and immobilized enzyme material may be mixed using any suitable means known in the art for about 10 hours or less, including from about 1.5 hours to about 10 hours.

In certain embodiments, the enzymatic treatment is complete in about 10 hours or less, including from about 1.5 hours to about 10 hours, or from about 1.5 hours to about 8 hours, or in about 10 hours, about 8 hours, about 6 hours, about 4 hours, or about 1.5 hours. In one particular embodiment, the enzymatic treatment is enzymatic interesterification. Reaction completion is achieved when the physical characteristics of the oil does not change with increasing reaction time. In one embodiment, reaction completion is based on complete randomization of the fatty acids on the triacylglycerols, and can be determined by measuring SFC, dropping point, slip point, and carbon number of the triglycerides in the oil, as described in the examples.

The lipid-containing compositions used in the processes of the present disclosure may be either crude oils; refined and bleached; refined, bleached, and either fully or partially hydrogenated; or fractionated, refined, bleached, and deodorized; or any combination thereof. Such compositions can comprise fats or oils from either vegetable sources or animal sources. If from vegetable sources, the oil or fat can be obtained by mechanical pressing or chemical extraction. Oils and fats suitable for use in the lipid-containing composition include, for example and without limitation, canola oil, castor oil, coconut oil, coriander oil, corn oil, cottonseed oil, hazelnut oil, hempseed oil, linseed oil, meadowfoam oil, olive oil, palm oil, palm kernel oil, peanut oil, rapeseed oil, rice bran oil, safflower oil, sasanqua oil, soybean oil, sunflower seed oil, tall oil, tsubaki oil, varieties of “natural” oils having altered fatty acid compositions via Genetically Modified Organisms (GMO) or traditional “breeding” such as high oleic or low linolenic, low saturated oils (high oleic canola oil, low linolenic soybean oil or high stearic sunflower oils), vegetable oil, menhaden, candlefish oil, cod-liver oil, orange roughly oil, sardine oil, herring oils, lard, tallow, algae oil, fish oil, animal-derived fat, waste cooking oil, brown grease, oil triglycerides derived from inedible plant sources, partial glycerides and free fatty acids derived from those oils or any mixture or blend of at least two thereof, at any desired ratio.

Although the methods of the present disclosure are directed to batch enzymatic treatment of a lipid-containing composition, it should be understood that the immobilized enzyme materials used in the processes of the present disclosure may also be used in connection with continuous flow applications. For instance, the immobilized enzyme materials may be used for the continuous enzymatic treatment of a lipid-containing composition using, for instance, a continuous flow reactor. Such a process involves charging the lipid-containing composition and immobilized enzyme material into a continuous flow reactor, such as a continuous stirred tank reactor (CSTR), a slurry bubble column or a continuous fixed bed reactor and known variations thereof. In one embodiment, the reactor is a continuous flow fixed bed reactor.

### Immobilized Enzyme Materials

For an immobilized enzyme to perform as an effective biocatalyst, a certain balance of properties is important. In particular, the carrier should possess certain properties to enable the enzyme to be immobilized with high loadings and a high retention of the enzyme's activity.

A suitable pore diameter is an important property of the carrier. The pore diameter must be of a suitable size to enable the enzyme to enter the pores to facilitate the immobilization of the enzyme throughout the pores of the carrier. Additionally, the carrier must possess a sufficiently high surface area to enable a high enzyme loading onto the surface of the carrier. The pore structure of the carrier should provide a favorable microenvironment without complications due to steric hindrance. The pore structure should result in low solution flow resistance and facilitate the mass transfer of reactants and products throughout the material.

Additionally, the chemical nature of the surface of the carrier needs to facilitate a high enzyme loading and in addition provide a chemical environment which enables retention of the enzyme on the surface of the carrier and furthermore ensures that the enzyme retains its activity once immobilized on the surface of the carrier.

Furthermore, for the enzymatic treatment of lipid-containing compositions, the carrier needs to be stable to the reaction conditions and avoid complications arising from swelling arising from contact with the lipids or chemical incompatibility with the lipids.

Thus, in general, to enable the immobilized enzyme to perform as an effective biocatalyst, a balance of properties such as surface area, pore volume, pore diameter, chemical-functionalization, and chemical stability, can provide in combination an effective biocatalyst suitable for industrial applications.

In one aspect, the immobilized enzyme material used in the methods of the present disclosure comprises:
a. a controlled porosity silica (CPS) as carrier, having a pore diameter from about 20 to about 100 nm; wherein the carrier comprises an amino-functionalized surface; and
b. one or more catalytically active enzyme(s) immobilized to the amino-functionalized surface by a covalent linker comprising a bond selected from amino, amide, ester, ether, imidoamide, imidothioamide, thioether, thioester and thioamide.

In a second aspect, the immobilized enzyme material comprises:
a. a controlled porosity silica (CPS) as carrier, having a pore diameter from about 20 to about 100 nm; wherein the carrier comprises an amino-functionalized surface; and
b. one or more catalytically active enzyme(s) immobilized to the surface via non-covalent interactions.

In a third aspect, the immobilized enzyme material comprises:
a. a controlled porosity silica (CPS) as carrier, having a pore diameter from about 20 to about 100 nm; wherein the carrier surface comprises at least two different coatings, of which at least one provides an amino functionalized surface; and
b. one or more catalytically active enzyme(s) immobilized on the surface, optionally by a covalent linker comprising a bond selected from amino, amide, ester, ether, thioether, imidoamide, imidothioamide, thioester and thioamide.

In a fourth aspect, immobilized enzyme material comprises:
a. a controlled porosity silica (CPS) as carrier, having a pore diameter from about 20 to about 100 nm; wherein the carrier comprises an amino-functionalized surface; and
b. one or more catalytically active enzyme(s) immobilized to the amino-functionalized surface, optionally by a covalent linker comprising a bond selected from amino, amide, ester, ether, thioether, imidoamide, imidothioamide, thioester and thioamide,
   wherein the enzyme(s) is/are selected from list (A), preferably from list (B) disclosed below.

Preferably, the covalent linker in the first, third and fourth aspects, when present, comprises a bond selected from the group consisting of amino, amide, ester, ether, thioether, thioester and thioamide. More preferably, the covalent linker in the first, third and fourth aspects, when present, comprises a bond selected from the group consisting of amino, amide, thioether, thioester and thioamide. Even more preferably, the covalent linker in the first, third and fourth aspects, when present, comprises a bond selected from the group of amino, amide, or thioamide. Yet more preferably, the covalent linker, in the first, third and fourth aspects, when present, comprises a bond selected from the group consisting of amino, amide, and imidoamide.

Preferably, the covalent linker in the first, third and fourth aspects immobilizing the enzymes(s) to the surface comprises a bond selected from amino and amide bonds, when present.

Without being bound by theory, the covalent linker between the amino-functionalized surface and the enzyme contains (i) a bond formed in the reaction between the amino-functionalized surface and a functional group on the cross-linking reagent used to form the covalent linker, (ii) a bond formed in the reaction between reactive functional groups in the enzyme and functional groups on the cross-linking reagent, and (iii) optionally, any spacer moiety that was present between the functional groups of the cross-linking agent, which contains preferably 3-20 atoms comprising any combination of C, N, H and O. The bond referred to as (i) preferably comprises a bond selected from the group consisting of amino, amide and imidoamide. The bond referred to as (ii) may comprise a bond selected from the group consisting of amino, amide, ester, ether, thioether, imidoamide, imidothioamide, thioester and thioamide.

Optionally, the immobilized enzymes may be intermolecularly covalently linked by a linker comprising a bond selected from amino, amide, ester, ether, thioester, imidoamide, imidothioamide, thioether and thioamide. These bonds are formed in the reaction between reactive functional groups in the enzyme and functional groups on the cross-linking reagent. The intermolecular linkers may optionally comprise a spacer moiety that was present between the functional groups of the cross-linking agent, which contains preferably 3-20 atoms comprising any combination of C, N, H and O.

The amino-functionalized surface comprises primary, secondary or tertiary amines.

The particulars and preferred features of the immobilized enzyme material are set out below.

### Carriers

Preferred carrier materials are inorganic particulate materials having a pore diameter from about 20 nm to about 100 nm, preferably from about 20 to about 60 nm, more preferably from about 30 to about 50 nm.

WO 2015/115993 describes chemically-coated controlled porosity glass materials as carriers for the preparation of immobilized enzymes with high enzyme loadings and high activities. The properties of the coated controlled porosity glass materials and the resultant biocatalysts as described in WO 2015/115993 are suitable for invention described herein, namely the enzymatic treatment of lipid-containing compositions.

Another class of carriers suitable for the invention described herein are controlled porosity silica (CPS) materials, and in particular, amino-functionalized CPS materials. The amino-functionalized CPS carriers allow immobilization of many different types of enzymes with high enzyme loadings, high retention of activity and high stability. The physical properties of amino-functionalized controlled porosity silica materials make them highly suitable for the immobilization of enzymes onto the surface of the carrier with high enzyme loadings and high enzyme activity retention.

The controlled porosity silica materials useful for the preparation amino-functionalized CPS materials are available commercially. For example, the Cariact class of controlled porosity silicas is available from Fuji Silysia Chemical Ltd. (https://www.fujisilysia.com/products/cariact/). These inorganic porous silicas can be produced with different surface areas and pore diameters. It has surprisingly been found that these materials are advantageous for the immobilization of enzymes with high enzyme loadings onto the surface of the carrier. The properties of Fuji Cariact Q-30 and Q-50 make these materials particularly advantageous. The materials Fuji Cariact Q-20C, Q30C and Q40C are also suitable. According to the manufacturer, the particles have the following characteristics:

| | surface | particle | | pore | pore |
|---|---|---|---|---|---|
| | area | sizes | | volume | diameter |
| Product | m²/g | available | | cm³/g | nm |
| Q-30 | 100 | 75-150 | μm | 1.00 | 30 |
| | | 75-500 | μm | | |
| | | 1.18-2.36 | mm | | |
| | | 1.70-4.00 | mm | | |
| Q-50 | 80 | 75-150 | μm | 1.00 | 50 |
| | | 75-500 | μm | | |
| | | 1.18-2.36 | mm | | |
| | | 1.70-4.00 | mm | | |
| Q-20C | 140 | 1.18-2.36 | mm | 0.80 | 20 |
| | | 1.70-4.00 | mm | | |
| Q-30C | 95 | 1.18-2.36 | mm | 0.80 | 30 |
| | | 1.70-4.00 | mm | | |
| Q-40C | 70 | 1.18-2.36 | mm | 0.80 | 40 |
| | | 1.70-4.00 | mm | | |

Another suitable commercially available material is Ecovyst E30. The particles have the following characteristics:

| | surface | particle | pore | pore |
|---|---|---|---|---|
| | area | diameter | volume | diameter |
| Material | m²/g | μm | cm³/g | nm |
| E30 | 114 | 70-220 | 0.93 | 32.6 |

Controlled porosity silica materials described herein can be prepared in accordance with the methods described in JPH0930809A and JP2020001936. Both methods describe the details of the production methods that provide sufficient control to modify the properties of the silica to produce controlled porosity silicas with the necessary balance of properties such as the previously specified surface areas, pore volumes, pore diameters and particle sizes. In addition, these production methods produce controlled porosity silica materials with the necessary mechanical strength to enable the features of the present disclosure. In general, two raw materials, sodium silicate and a mineral acid such as sulphuric acid, are used together in a solution process to generate monomeric silicic acid. Monomeric silicic acid polymerizes to generate primary silica particles, referred to as silica sol. These primary particles then aggregate together to form a three-dimensional porous structure. The reaction conditions that enable the growth of the primary particles and conditions to dry the product are used to control and to modify physical properties such as surface area, pore diameter and pore volume.

The controlled porosity silica materials described herein have been specifically designed for use as catalyst supports where mechanical properties such as crush strength, attrition and abrasion resistance are a necessary feature of the material for performance as catalyst supports. The controlled porosity silica materials, which are chemically and thermally stable, also provide precisely controlled porosity diameters and pore diameter distributions. Particle sizes and particle size ranges can be tailored and optimized for different industrial reactor types. For example, they may be deployed in batch or continuous stirred tank reactors (CSTR) as slurry catalysts or slurry bubble columns or fixed bed reactors or known variations thereof.

Suitably sized pores provide a favorable microenvironment without complications due to steric hindrance. The pore structure results in low solution flow resistance and facilitates the mass transfer of reactants and products throughout the material. The rigid non-caged structure of CPS provides a rugged, noncompressible medium suitable for high throughput reactor designs and linear scale up at high flow rates. The material displays limited swelling in solvents and is chemically and dimensionally stable in most organic media and aqueous environments at pH below 10.

In general, controlled porosity silica carriers that are suitable for the invention have pore diameters from about 20 nm to about 100 nm, preferably from about 20 to about 60 nm, more preferably from about 30 to about 50 nm. Preferably, the surface areas from about 50 m²/g to about 200 m²/g (preferably greater than about 60 m²/g, such as greater than about 70 m²/g). Also preferably, the pore volume is from about 0.5 mL/g to about 2.0 mL/g (more preferably at least about 0.6 mL/g, even more preferably at least about 0.7 mL/g, and most preferably about 0.8 mL/g to about 1.5 mL/g). These properties allow the immobilization of enzymes with high loadings onto the surface of the carrier. Preferably, the pore diameter of the carrier is from about 20 to about 60 nm and the surface area is from about 50 m²/g to about 200 m²/g. Even more preferably, the carrier has a pore diameter from about 20 to about 60 nm, a surface area from about 50 m²/g to about 200 m²/g and a pore volume of from about 0.5 mL/g to about 2.0 mL/g. Most preferably, the carrier has a pore diameter from 20 to 60 nm, a surface area from 50 m²/g to 200 m²/g and a pore volume of from 0.5 mL/g to 2.0 mL/g. The ranges disclosed herein include the endpoints.

A CPS support exhibits an enzyme loading capacity that is inversely related to its pore diameter. Thus, a CPS support of a large pore diameter cannot be loaded with as much enzyme as a CPS support of a smaller pore diameter, which is largely due to the inverse relationship between pore diameter and surface area (i.e., a lower available surface area for enzyme immobilization).

The preferred particle size of the carrier is dependent on the type of reactor deployed for the use of the immobilized enzyme material. For example, smaller particles sizes, e.g., 75-150 μm are well suited for the deployment of a batch or a continuous stirred tank reactor where the immobilized enzyme material operates as a catalyst slurry in the reactor. For deployment in a fixed bed reactor, the preferred particle size is dependent on the chemical reaction and the conditions within the reactor. Smaller particles e.g., 75-150 μm are preferred for liquid phase fixed bed reactions whereas larger particle sizes can be deployed for certain other liquid phase reactions and reactions where a gas and a liquid are deployed together within the fixed bed reactor, e.g., particle sizes greater than 200 μm, and preferably greater than 500 μm.

The amino-functionalized CPS carriers are chemically stable, meaning that they can be utilized in a broad range of reaction conditions including reactions containing a substrate such as a lipid.

It should be understood that CPS in the present context is distinct from siliceous mesocellular foams (MCFs) known in the literature. MCFs possess ordered 3D cage-like structures with spherical cavities having diameters of 20-40 nm, interconnected by pores of around 10 nm in size, which are lower than desired for the immobilisation of enzymes. In contrast, CPS is not a caged structure. In the context of MCF materials, literature often uses the terms pore size to define the diameter of the cage and pore window to define the cage entrance diameter (or pore diameter) where the window size is typically smaller than the pore size. Due to the cage-like structure and lower window sizes, the surface areas (m²/g) of MCF materials are typically significantly higher than that of CPS (over 200 m²/g). MCF is currently not commercially available at industrial scale and is prohibitively expensive for industrial applications, so it is not a practically viable alternative to CPS.

Controlled porosity silicas are also distinct from hollow microsphere silicas described in the literature. WO 2013/078551 describes hollow microsphere silicas as microcapsules comprising a silica shell having a thickness of from about 50 nm to about 500 μm, said shell surrounding a hollow capsule having a diameter from about 0.1 μm to about 1500 μm, and having a density of about 0.001 g/cm³ to about 1.0 g/cm³. The hollow feature of these materials is shown clearly in FIGS. 2-5 of WO 2013/078551. These materials are core/shell/functional surface type reservoirs or microcapsules, comprising a core (gaseous or hollow) surrounded by a shell (generally solid) composed essentially of one or more silica-based materials and capped with a functional surface with affinity or adhesion to the matrix of plastics or composites or rubbers or textiles. The microcapsules are designed to be introduced into plastics, composites, rubbers and textiles products in their processing stage. Gaseous or hollow microcapsules are dispersed throughout or partially in plastics, composites, rubbers and textiles products as a density-reducing additive to reduce the density of the final products. Whereas the hollow sphere microcapsules of WO 2013/078551 are designed as a density reducing additive that can provide a material benefit to a formulated product, the controlled porosity silica materials of the invention have been specifically designed for use as catalyst supports where mechanical properties such as crush strength, attrition and abrasion resistance are a necessary feature of the material for performance as catalyst. Such properties would not be provided by such low-density, hollow sphere supports with an inherently low crush strength. The controlled porosity silicas of the invention are not hollow and comprise a three-dimensional silica structure comprising precisely controlled pore diameters and pore diameter distributions throughout the entire solid well suited to the synthesis of efficient and robust biocatalysts.

### Amino Functionalized Surface

The amino-functionalized surface may comprise a structure of formula (I), (II), or (III):

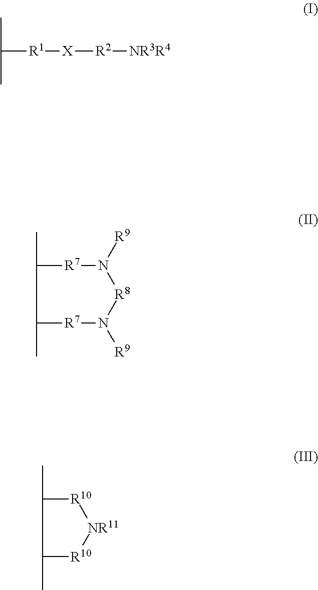

wherein
R¹ is C₁₋₆ alkanediyl; and wherein R¹ provides a covalent attachment to the silica surface (the vertical line on the left);
X is a bond (i.e., X is absent) or is a phenyl ring;
R² is C₁₋₆ alkanediyl;
R³ is W;
R⁴ is W or is selected from the group consisting of C₁₋₁₀ alkyl, C₅₋₇ cycloalkyl, phenyl, phenyl-C₁₋₆-alkyl, amino-C₂₋₈ alkyl, N-(phenyl)amino-C₂₋₈ alkyl and N-(phenyl-C₁₋₆-alkyl)amino-C₂₋₈ alkyl, and aminocarbonyl; and wherein phenyl, at each instance, is optionally substituted with one or more substituents C₁₋₄ alkyl;
or R³ and R⁴, together with the nitrogen atom to which they are attached, combine to form a 5- to 7-membered heterocyclic ring which may contain one or more unsaturated bonds and which may contain one more additional heteroatoms, on which a covalent linker comprising a bond selected from amino, amide, and imidoamide is optionally attached, wherein a catalytically active enzyme also is attached to the linker; or R³ and R⁴ together form ═CR⁵R⁶, wherein R⁵ and R⁶ are each independently C₁₋₄ alkyl;
each R⁷ is independently C₁₋₆ alkanediyl; and wherein R⁷ provides a covalent attachment to the silica surface (the vertical line on the left);
R⁸ is C₁₋₃ alkanediyl;
each R⁹ is independently W or is selected from the group consisting of C₁₋₄ alkyl and hydroxy-C₁₋₄ alkyl;
each R¹⁰ is independently C₁₋₆ alkanediyl; and wherein R¹⁰ provides a covalent attachment to the silica surface (the vertical line on the left);
R¹¹ is W or is selected from the group consisting of C₁₋₁₀ alkyl, C₅₋₇ cycloalkyl, phenyl, phenyl-C₁₋₆-alkyl, amino-C₂₋₈ alkyl, N-(phenyl)amino-C₂₋₈ alkyl and N-(phenyl-C₁₋₆-alkyl)amino-C₂₋₈ alkyl, and aminocarbonyl; and wherein phenyl, at each instance, is optionally substituted with one or more substituents C₁₋₄ alkyl; and
each W is independently hydrogen or a covalent linker comprising a bond selected from amino, amide, and imidoamide, wherein a catalytically active enzyme is also attached to the linker.

In some embodiments, the amino-functionalized surface comprises a structure of formula (I), as defined above, in combination with a structure of formula (IV):

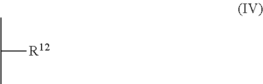

wherein R¹² is selected from C₁₋₁₂ alkyl and amino-C₁₋₁₂ alkyl. Preferably, the amino functional groups in the surface are not part of an aromatic heterocyclic ring, more preferably not any heterocyclic ring. Preferably, the surface does not contain any functional groups that may chelate a metal ion apart from the amino groups. More preferably, the surface comprises amino-functionalized aliphatic moieties.

In some instances, it is beneficial to have at least two different coating on the carrier surface (of which at least one providing the amino-functionalization). The presence of two different coatings can provide, for certain enzymes, a better balance of properties suited to the said certain enzyme, leading to a more efficient biocatalyst. For example, a certain enzyme may prefer a coating which is more hydrophobic in nature and may also benefit from the presence of an amino group, and additionally a metal ion. Such a situation can be accommodated by the presence of at least two coatings, one providing the amino-functionalization and the other providing a hydrophobic coating. The hydrophobic coating may comprise alkyl moieties and/or aromatic moieties. Preferably, the hydrophobic coating comprises C₁₋₁₂ alkyl groups, preferably C₁₋₆ alkyl groups and/or a phenyl group.

For instance, one coating may contain an aliphatic chain, or a phenyl group, providing a hydrophobic environment, and the second may contain an amino-functionalized group, which may further accommodate a metal ion. The relative proportions of the two coatings on the surface can be controlled and optimized to maximize the efficiency of the biocatalyst. The relative amount of the two coatings may be in the range of 9:1 to 1:9 on weight basis, preferably 5:1 to 1:5, more preferably 2:1 to 1:2, most preferably 1:1.

Furthermore, the presence of two coatings can benefit the situation where a covalent linkage is preferred between the enzyme and the surface of the CPS material. The presence of two coatings can tailor the surface with two coatings that can a) provide a chemical group that is preferred by the enzyme, for example a hydrophobic group such as an aliphatic chain or a phenyl group, and b) provide an amino-functionalized group to enable a covalent linkage between the enzyme and the CPS surface.

Preferably, the amino-functionalized surface comprises any of the following structures wherein each W is hydrogen or a covalent linker comprising a bond selected from amino, amide, and imidoamide, wherein a catalytically active enzyme is also attached to the linker:

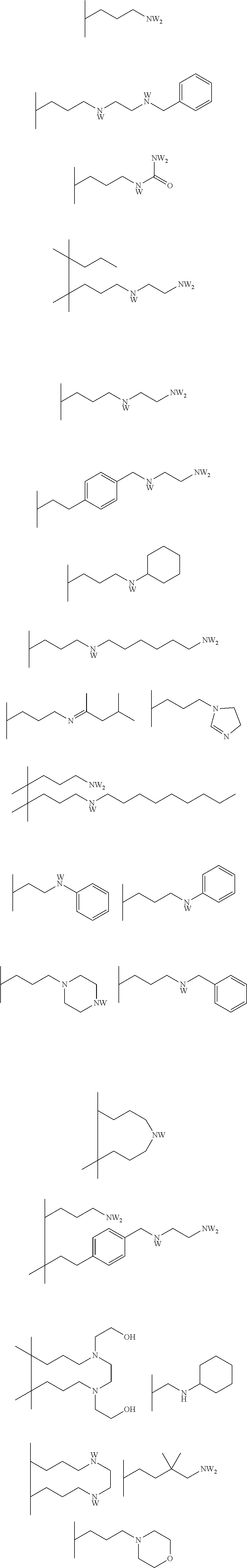

In some embodiments, the amino-functionalized surface comprises structure (I′):

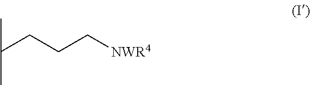

wherein
R⁴ is W or is selected from the group consisting of phenyl, phenyl-C₁₋₆-alkyl, amino-C₂₋₈ alkyl, N-(phenyl)amino-C₂₋₈ alkyl and N-(phenyl-C₁₋₆-alkyl)amino-C₂₋₈ alkyl, and wherein phenyl, at each instance, is optionally substituted with one or more substituents C₁₋₄ alkyl; and
W is hydrogen or a covalent linker comprising a bond selected from amino, amide, and imidoamide, wherein a catalytically active enzyme is also attached to the linker;
   or wherein the amino-functionalized surface comprises structure (I″):

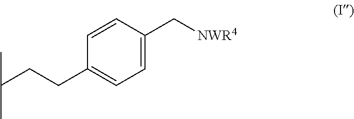

wherein
R⁴ is W or is selected from the group consisting of amino-C₂₋₈ alkyl, N-(phenyl)amino-C₂₋₈ alkyl and N-(phenyl-C₁₋₆-alkyl)amino-C₂₋₈ alkyl, and wherein phenyl, at each instance, is optionally substituted with one or more substituents C₁₋₄ alkyl; and
W is hydrogen or a covalent linker comprising a bond selected from amino, amide, and imidoamide, wherein a catalytically active enzyme is also attached to the linker.

In certain preferred embodiments, the amino-functionalized surface comprises structure I′ wherein R⁴ is W or is selected from the group consisting of phenyl, benzyl, amino-C₂₋₆ alkyl, N-(phenyl)amino-C₂₋₆ alkyl and N-(benzyl)amino-C₂₋₆ alkyl; or the amino-functionalized surface comprises structure I″ wherein R⁴ is W.

In some embodiments, the amino-functionalized surface comprises any of the following structures, wherein each W is hydrogen or a covalent linker comprising a bond selected from amino, amide, and imidoamide, wherein a catalytically active enzyme is also attached to the linker:

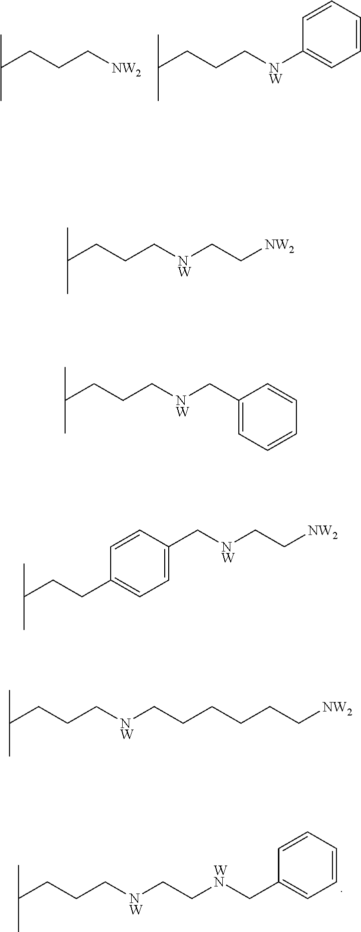

Most preferably, the amino-functionalized surface comprises any of the following structures, wherein each W is hydrogen or a covalent linker comprising a bond selected from amino, amide, and imidoamide, wherein a catalytically active enzyme is also attached to the linker:

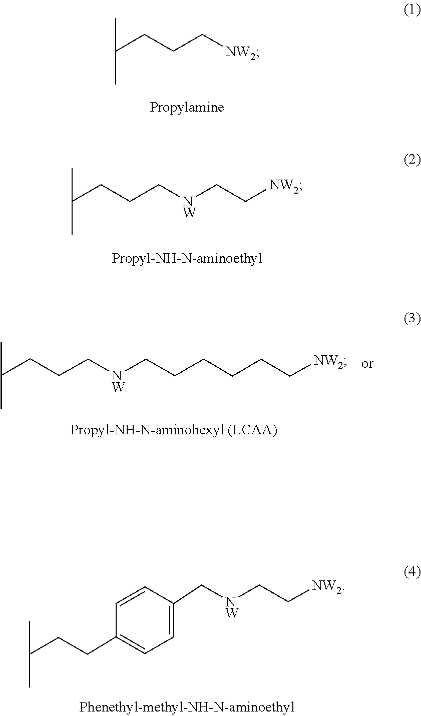

For avoidance of any doubt, it should be understood that the above names for structures (1)-(4) are derived from the situation where W═H, but the structures nonetheless also encompass the situation where W is a covalent linker comprising a bond selected from amino, amide, and imidoamide, wherein a catalytically active enzyme is also attached to the linker.

As used herein, the term “C₁₋₆ alkanediyl” refers to a straight or branched divalent group having from 1 to 6 carbon atoms. Examples of C₁₋₆ alkanediyl include methanediyl, ethanediyl, 1,3-propanediyl and 2,2-dimethyl-1,4-butanediyl.

As used herein, the term “C₁₋₁₀ alkyl” refers to a straight or branched alkyl group having from 1 to 10 carbon atoms, and the term “C₁₋₄ alkyl” refers to a straight or branched alkyl group having from 1 to 4 carbon atoms. Examples of C₁₋₄ alkyl include methyl, ethyl, n-propyl, isopropyl, n-butyl, isobutyl, sec-butyl and tert-butyl.

As used herein, the term “C₅₋₇ cycloalkyl” refers to a monocyclic saturated hydrocarbon ring having from 5 to 7 carbon atoms. Examples of C₅₋₇ cycloalkyl include cyclopentyl, cyclohexyl and cycloheptyl.

As used herein, the term “hydroxy-C₁₋₄ alkyl” refers to a C₁₋₄ alkyl group wherein one of the hydrogen atoms is replaced with a hydroxy group.

As used herein, the term “phenyl-C₁₋₆ alkyl” refers to a C₁₋₆ alkyl group wherein one of the hydrogen atoms is replaced with a phenyl group.

As used herein, the term “amino-C₂₋₈ alkyl” refers to a C₂₋₈ alkyl group wherein one of the hydrogen atoms is replaced with amino.

The term “amino” refers to primary, secondary and tertiary amines.

The term “aminocarbonyl” refers to a group —C(═O)NH₂.

As used herein, the terms “N-(phenyl)amino-C₂₋₈ alkyl” and “N-(phenyl-C₁₋₆-alkyl)amino-C₂₋₈ alkyl” refer to an amino-C₂₋₈ alkyl group wherein one of the hydrogen atoms on the amino group is replaced with a phenyl or a phenyl-C₁₋₆ alkyl group, respectively.

The term surface as used herein in the context of the immobilized enzyme material described herein refers to the entire surface of the CPS carrier and includes both the outer surface and the surface of the pores. Due to the nature of the CPS, the greater part of the surface area is present in the pores.

### Immobilized Enzymes

Any enzyme of interest can be used in the processes of the current disclosure. For example, the enzymes used in the processes of the present invention can be a lipase; esterase; acylase; transferase; oxidoreductase; hydrolase; ligase; isomerase; lyase; those enzymes that facilitate acidolysis reactions, transesterification reactions, ester synthesis, or ester interchange reactions; enzymes having phospholipase or protease activity, including thermostable and thermotolerant hydrolase activity; and polynucleotides. Suitable enzymes include, without limitation, those derived from of Achromobacter, Alcaligenes, Aspergillus, Bacillus, Burkholderia, Candida, Chromobacterium, Corynebacterium, Fusarium, Geotrichum, Humicolo, Humicora, Mucor, Penicillium, Pseudomonas, Rhizomucor, Rhizopus, Staphylococcus, Thermomyces, or Torulopsis. Suitable derived enzymes include without limitation Aspergillus niger Mucor mihei, Mucor javanicus Pseudomonas fluorescens, Rhizopus delemar, Burkholderia cepacia, Candida cylindracea, Candida Antarctica, Candida rogosa, Fusarium solani, Penicillium cyclopium, Rhizomucor miehei and Thermomyces lanuginosus.

In one embodiment, the immobilized enzyme(s) may be selected from enzymes on list (A) consisting of items 1-62 in the following table A and their enzymatically active sequence variants, preferably variants having similar enzymatic activity, and most preferably substantially the same enzymatic activity.

It is well known that the amino-acid sequence of an enzyme can be varied considerably without significantly affecting the enzymatic activity. For instance, artificial sequences such as His-tags can be added to enable metal chelation. In other instances, residues that are not critical to enzymatic activity can be modified to improve properties (such as stability or efficiency of production) or simply to provide an alternative sequence to circumvent patent protection. In some cases, the enzyme sequence can be truncated to produce a more compact enzyme by dispensing with parts of the enzyme not critical to enzyme activity. Thus, enzymatically active sequence variants of the enzymes identified in table A can also be used and are therefore encompassed by list (A).

TABLE A
preferred immobilized enzymes

| # | Enzyme name (organism) | Uniprot id | EC Number |
|---|---|---|---|
| 1 | Lipase (Thermomyces lanuginosus) | O59952 | 3.1.1.3 |
| 2 | Lipase CALB (Pseudozyma antarctica) | P41365 | 3.1.1.3 |
| 3 | Sucrose phosphorylase (Bifidobacterium adolescentis) | A0ZZH6 | 2.4.1.7 |
| 4 | Isopropanol dehydrogenase (Thermoanaerobacter brockii) | P14941 | 1.1.1.80 |
| 5 | Fructose-6-phosphate aldolase (Escherichia coli) | P78055 | 4.1.2.— |
| 6 | Phenylalanine ammonia-lyase (Petroselinum crispum) | P24481 | 4.3.1.24 |
| 7 | Xylose isomerase (Thermotoga neapolitana) | P45687 | 5.3.1.5 |
| 8 | Triosephosphate isomerase (Escherichia coli) | P0A858 | 5.3.1.1 |
| 9 | Glucose oxidase (Aspergillus niger) | P13006 | 1.1.3.4 |
| 10 | Cholesterol oxidase (Streptomyces sp) | P12676 | 1.1.3.6 |
| 11 | Galactose Oxidase (Gibberella zeae) | P0CS93 | 1.1.3.9 |
| 12 | Choline oxidase (Arthrobacter globiformis) | Q7X2H8 | 1.1.3.17 |
| 13 | Alcohol oxidase (Candida boidinii) | Q00922 | 1.1.3.13 |
| 14 | L-alanine dehydrogemase (Bacillus subtilis) | Q08352 | 1.4.1.1 |
| 15 | Alcohol dehydrogenase (Saccharomyces cerevisia) | P00330 | 1.1.1.1 |
| 16 | Glucose-6-phosphate Dehydrogenase (Leuconostoc | P11411 | 1.1.1.363 |
| | mesenteroides) | | |
| 17 | Glycerol dehydrogenase (Citrobacter freundii) | P45511 | 1.1.1.6 |
| 18 | Sucrose phosphorylase (Leuconostoc mesenteroides) | Q59495 | 2.4.1.7 |
| 19 | Formate dehydrogenase (Candida boidinii) | O13437 | 1.17.1.9 |
| 20 | Phosphonate dehydrogenase (Pseudomonas stutzeri) | O69054 | 1.20.1.1 |
| 21 | Glucose dehydrogenase (Bacillus megaterium) | P39484 | 1.1.1.47 |
| 22 | Glucose dehydrogenase (Thermoplasma acidophilum) | P13203 | 1.1.1.47 |
| 23 | Laccase (Bacillus subtilis) | P07788 | 1.10.3.2 |
| 24 | Alpha-galactosidase (Thermotoga maritima) | G4FEF4 | 3.2.1.22 |
| 25 | Beta-galactosidase (Bifidobacterium adolecentis) | A1A399 | 3.2.1.23 |
| 26 | Beta-galactosidase (Aspergillus oryzae) | Q2UCU3 | 3.2.1.24 |
| 27 | Endo-1,4-beta-xylanase (Aspergillus oryzae) | Q9HFA4 | 3.2.1.8 |
| 28 | Alpha-amylase (Bacillus licheniformis) | P06278 | 3.2.1.1 |
| 29 | Xylose isomerase (Streptomyces rubiginosus) | P24300 | 5.3.1.5 |
| 30 | Phosphoglucomutase (Thermococcus kodakarensis) | Q68BJ6 | 5.4.2.2 |
| 31 | Glucose-6-phosphate isomerase (Thermotoga maritima) | Q9X1A5 | 5.3.1.9 |
| 32 | Cellulase (Acetivibrio thermocellus) | P10477 | 3.2.1.4 |
| 33 | Cellulase (Thermoanaerobacterium) | A0A0U4EBH5 | 3.2.1.4 |
| 34 | Acid phosphatase (Salmonella typhimurium) | P26976 | 3.1.3.2 |
| 35 | Polyphosphate--nucleotide phosphotransferase | A0A3D5XRJ5 | 2.7.4.1 |
| | (Erysipelotrichaceae bacterium) | | |
| 36 | Amylosucrase (Neisseria polysaccharea) | Q9ZEU2 | 2.4.1.4 |
| 37 | Glutamate--cysteine ligase (Escherichia coli) | P0A6W9 | 6.3.2.2 |
| 38 | Glycerol kinase (Escherichia coli) | P0A6F3 | 2.7.1.30 |
| 39 | Aminotransferase (Chromobacterium violaceum) | Q7NWG4 | 2.6.1.62 |
| 40 | P450 (Bacillus megaterium) | P14779 | 1.6.2.4 |
| 41 | Acid sugar phosphatase (Bacillus subtilis) | O32125 | 3.1.3.5 |
| 42 | D-xylonate dehydratase (Caulobacter vibrioides) | Q9A9Z2 | 4.2.1.82 |
| 43 | UDP-glucose 4-epimerase (Chloroflexi bacterium) | A0A7C4N9I7 | 5.1.3.2 |
| 44 | Alkylhalidase (Aspergillus niger) | A2QL40 | 3.8.1.1 |
| 45 | D-amino acid oxidase (Sus scrofa) | P00371 | 1.4.3.3 |
| 46 | Adenylate kinase (Homo sapiens) | P13569 | 2.7.4.3 |
| 47 | Alanine racemase (Mycobacterium tuberculosis) | P9WQA9 | 5.1.1.1 |
| 48 | Haloalkane dehalogenase (Xanthobacter autrophicus) | P22643 | 3.8.1.5 |
| 49 | Aromatic peroxygenase (Agrocybe aegerita) | B9W4V6 | 1.11.2.1 |
| 50 | Acetase kinase (Bacillus subtilis) | P37877 | 2.7.2.1 |
| 51 | Fatty acid peroxygenase (Bacillus subtilis) | O31440 | 1.11.2.4 |
| 52 | Glycerol dehydratase (Citrobacter freundii) | P45514 | 4.2.1.30 |
| 53 | Carbonic anhydrase (Gallus gallus) | P07630 | 4.2.1.1 |
| 54 | Catalase (Bacillus subtilis) | P94377 | 1.11.1.6 |
| 55 | Fructokinase (Bacillus subtilis) | O05510 | 2.7.1.4 |
| 56 | NAD(P)H oxidase (Sus scrofa) | Q8HZK2 | 1.6.3.1 |
| 57 | NAD(P)H oxidase (Thermococcus kodakarensis) | Q5JGP4 | 1.6.3.2 |
| 58 | Hexokinase (Saccharomyces cerevisiae) | P04806 | 2.7.1.1 |
| 59 | Amidase (Arabidopsis thaliana) | Q9FR37 | 3.5.1.4 |
| 60 | Aminotransferase (Arthrobacter species) | F7J696 | 2.6.1.18 |
| 61 | Amyloglucosidase 300L (Aspergillus niger) | Ref 1 | 3.2.1.3 |
| 62 | Aminotransferase ATA_633 (Arthrobacter species) | Ref 2 | 2.6.1.26 |

Ref 1: Global Substance Registration System Ver 3.0.3 UNII record A370TYK9KO
Ref 2: Cuetos et al. (2016) Angew Chem Int Ed 2016, 55: 3144-3147

The immobilized enzyme(s) is/are preferably selected from list (B) consisting of: Thermomyces laguginosus lipase (TLL, #1 in Table A), Candida antarctica lipase (CalB, #2 in Table A), Bifidobacterium adolescenti sucrose phosphorylase (SucP, #3 in Table A), Thermoanaerobacter brockii secondary alcohol dehydrogenase (TbSADH, #4 in Table A), Arthrobacter species amine transaminase (ATA, #60 in Table A), Leuconostoc mesenteroides glycosyl transferase (GT, #18 in Table A), amyloglucosidase from Aspergillus niger (#61 in Table A), and aminotransferase (Arthrobacter species) (#62 in Table A); and their enzymatically active sequence variants, preferably variants having similar enzymatic activity, and most preferably substantially same enzymatic activity.

A particularly preferred enzyme catalyst is the lipase from Thermomyces lanuginosus. A Thermomyces lanuginosus lipase having SEQ ID NO: 1 is available in liquid form as Lipozyme® 100 L (Novozymes).

The enzyme activity relevant in the context of lists (A) and (B) is indicated in table A by the “EC Number” column, which refers to the numerical classification scheme of the Enzyme Commission, which categorizes enzymes by their catalyzed reactions.

The Uniprot id-column in table (A) refers to entries on the public Uniprot database at www.uniprot.org, as of 1 Dec. 2021. Preferably, each selected enzyme of list (A) or list (B) has at least 70% sequence identity to the sequence according to the database entry identified by the “Uniprot id” indicated in table A, more preferably at least 80%, even more preferably at least 90%, still more preferably at least 95% and most preferably complete sequence identity.

In some embodiments, the enzyme comprises a metal affinity tag such as a polyhistidine tag, HQ-tag, MAT Tag, or any other suitable tags known in the art, to facilitate immobilization via metal chelation.

### Enzyme Immobilization

In preferred embodiments of the third and fourth aspects, the enzyme(s) is/are immobilized to the surface by a covalent linker comprising a bond selected from amino, amide, and imidoamide, although non-covalent immobilization is also an option.

In preferred embodiments of the first, third and fourth aspects, the enzyme(s) is/are immobilized to the surface by a covalent linker comprising a bond selected from amino, amide, and imidoamide, and prior to formation of the bond, the enzyme(s) had been immobilized to the surface via non-covalent interactions, preferably via an interaction mediated by a chelated metal ion. Without being bound by theory, immobilization after non-covalent binding may allow for more uniform cross-link formation between the CPS support surface and the enzyme, resulting in improved retention of activity. In other embodiments, the initial immobilization is through covalent linkers, optionally additionally followed by intermolecular cross-linking.

In preferred embodiments of the second, third and fourth aspects, the enzyme(s) is/are immobilized to the surface non-covalently via an interaction mediated by a chelated metal ion.

The chelated metal ion referred to above may be selected from Ni²⁺, Cu²⁺, Mg²⁺, Fe³⁺ or Zn²⁺. Preferably, the metal ion is a Zn²⁺ ion. Such Zn²⁺ ion mediated interaction provides superior results for certain enzymes such as CalB and TbSADH.

In certain embodiments of the first, second, third or fourth aspects, the immobilized enzyme(s) are intermolecularly covalently linked by a linker comprising a bond selected from amino, amide, ester, ether, thioether, imidoamide, imidothioamide, thioester and thioamide.

Preferably, the covalent linker between the surface and the enzyme(s), when present, does not comprise imine bonds, which can be susceptible to cleavage by hydrolysis. Preferably, the intermolecular covalent linker, when present, does not comprise imine bonds. Preferably, the covalent linker, when present, comprises a bridge containing between 3 and 20 atoms comprising any combination of C, H, N and O.

In some embodiments, the enzyme comprises a metal affinity tag such as a polyhistidine tag (His-tag of 2-8 consecutive histidines, preferably 6), HQ-tag, MAT Tag, or any other suitable tags known in the art, to facilitate immobilization via metal chelation. It should be noted that many enzymes of industrial relevance can be immobilized via metal chelation even without an affinity tag, due to innate metal-chelating properties of the enzyme. An advantage of immobilization via metal chelation using an affinity tag, however, is selective enzyme binding when compared to physical adsorption, and in addition the steric orientation of the immobilized enzyme may be more uniform compared to the situation where the immobilization occurs for example via non-specific physical adsorption. An affinity tag may also improve activity after immobilization by driving the immobilization in a controlled position allowing better and/or more consistent access to the active site. On the other hand, initial immobilization through covalent linkers can also apply as it can afford a simplified manufacturing process since the step of non-covalent immobilization can be omitted.

Covalent cross-linking (between different enzyme molecules and/or between enzyme molecules and the amino groups of the amino-functionalized surface) has certain advantages. The cross-linking allows better retention of enzyme activity in industrial flow conditions, and the possibility to recycle and reuse the catalyst in batch operations.

Suitable cross-linking reagents for formation of the covalent cross-links are disclosed below.

### Cross-Linking Reagents

Various covalent cross-linking strategies as applied to amino functional groups and enzymes are known in the field of protein chemistry. See for example, the Crosslinking Technical Handbook published by Thermo Scientific incorporated herein by reference, https://tools.thermofisher.com/content/sfs/brochures/1602163-Crosslinking-Reagents-Handbook.pdf.

An effective cross-linking reagent that links an enzyme to the amino-functionalized carrier surface will require at least two (preferably no more than two) reactive groups separated by a linker. An effective cross-linking reagent needs to contain at least a first reactive group that can react with the amino functional group attached to the CPS surface to form a stable covalent bond. Additionally, the cross-linking reagent needs to contain at least a second reactive group that can react with a functional group that is present on the enzyme structure to form a stable covalent bond, thus creating a covalent link between the enzyme and the support surface. Alternatively, the reagent can contain at least two reactive groups that each can react with a functional group that is present on the enzyme structure to form a stable covalent bond, thus creating an intermolecular covalent link between enzyme molecules.

Functional groups on enzymes that are amenable to cross-linking include amine groups, carboxylate groups, thiol (sulfhydryl) and hydroxy groups. With an appropriate selection of a cross-linking reagent, these functional groups can readily form covalent bonds such as amino, amide, ester, ether, thioether, imidoamide, imidothioamide, thioester, and thioamide. As a specific example, in the case of an epoxide cross-linking reagent, the functional groups on the enzymes can react with an epoxide group to produce covalent bonds such as amino, ester, ether and thioether bonds. For example, in case of bis-epoxide cross-linking reagents, one of the epoxide groups can react with the amino functional group situated on the surface of the amino-functionalized controlled porosity silica carrier to form an amino covalent bond. The second epoxide group can react with a functional group situated on an amino acid residue located on the enzyme. For example, an amino functional group from a lysine amino acid residue can react with the epoxide group to form an amino covalent bond. Alternatively, a thiol functional group from a cysteine amino acid residue can react with the epoxide group to form a thioether covalent bond. In yet further embodiments, a carboxylate functional group from a glutamate amino acid residue can react with the epoxide group to form an ester covalent bond or a hydroxyl group from a serine amino acid residue can form an ether covalent bond.

The cross-linking reagents may similarly be used for forming intermolecular linkers between immobilized enzymes, when desired.

The cross-linking reagent can be homobifunctional or heterobifunctional, meaning that the first and second reactive groups can either be the same or different. Preferably, the first and second reactive groups are separated by a linker containing between 3 and 20 atoms comprising any combination of C, H, N and O.

An effective cross-linker should create a covalent link between the enzyme and the support surface and retain the enzyme activity upon cross-linking. Retention of the enzyme activity upon cross-linking is an important desirable feature of a cross-linking reagent which can be tuned with a suitable choice of the reactive groups, the linker group, and the linker length.

The preferred reactive groups selected are chosen for their capability for reacting with amines, hydroxyls, carboxylates or sulfhydryls to create stable covalent linkages via amino, amide, ether, ester, imidoamide, imidothioamide, thioether, thioester or thioamide linkages. A cross-linking reagent capable of cross-linking with the functional groups of the enzyme may contain at least two reactive groups independently selected from epoxides, esters, anhydrides, N-hydroxysuccinimide esters, imidoesters, carbonates, acylisoureas, carbodiimides, maleimides, haloacetyls, thiosulfonates, isocyanates, and vinylsulfones. Reactive groups such as carbodiimides can assist in the formation of a covalent linkage by reacting with, for example, a carboxylate group and then itself being subject to a displacement by a second reactive group to form a covalent bond such as an amide bond.

Preferred cross-linking reagents are defined by the formula below:

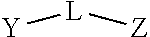

where Y and Z are independently a reactive group selected from an epoxide, ester, anhydride, imidoester, N-hydroxysuccinimide ester, carbonate, acylisourea, a carbodiimide, a maleimide, a haloacetyl, a thiosulfonate, a vinylsulfone and an isocyanate, and preferably, Y and Z are each independently epoxide, N-hydroxysuccinimide ester or imidoester;
L represents a linker between the reactive groups containing between 3 and 20 atoms comprising any combination of C, H, N and O.

More preferred cross-linking reagents are homobifunctional where Y═Z and contain reactive groups selected from an epoxide, ester, anhydride, N-hydroxysuccinimide ester, imidoester, carbonate, acylisourea, maleimide, haloacetyl and vinylsulfone, most preferably epoxide, N-hydroxysuccinimide ester or imidoester.

Even more preferably the cross-linking reagent is a bis-epoxide reagent, still more preferably a bis-epoxide defined by the following formula:

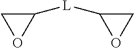

where L represents a linker between the epoxide groups containing between 3 and 20 atoms comprising any combination of C, H, N and O.

Most preferably the bis-epoxide reagent is glycerol diglycidyl ether (GDE, which can be a 1,3- or 1,2-substituted glycerol diglycidyl ether, or a mixture of both isomers), neopentyl glycol diglycidyl ether (NPE), poly(tetra ethylene oxide) diglycidyl ether (PDE), 1,6-hexanediol diglycidyl ether (HDDE) or glycerol triglycidyl ether (GTGE).

Alternative preferred cross-linking reagents include bifunctional sulfonated N-hydroxysuccinimide esters and bifunctional imidoesters such as bis(sulfosuccinimidyl)suberate (BS3) or dimethyl suberimidate (DMS).

A known alternative cross-linker is glutaraldehyde, but it results in the formation of imine bonds so it is not encompassed by the present invention. Glutaraldehyde is a particularly reactive cross-linking reagent that leads to a significant degree of enzyme inactivation. The imine bond formation is also reversible so the links are not very stable.

### Preferred Properties of the Immobilized Enzyme Materials

In a preferred embodiment of the first aspect, the support material has a pore diameter from about 20 to about 60 nm, a surface area from about 50 m²/g to about 200 m²/g and a pore volume of from about 0.5 mL/g to about 1.5 mL/g, and the enzyme(s) is/are selected from list (A), or even more preferably from list (B).

In a preferred embodiment of the second aspect, the support material has a pore diameter from about 20 to about 60 nm, a surface area from about 50 m²/g to about 200 m²/g and a pore volume of from about 0.5 mL/g to about 1.5 mL/g, and the enzyme(s) is/are selected from list (A), or even more preferably from list (B).

In a preferred embodiment of the third aspect, the support material has a pore diameter from about 20 to about 60 nm, a surface area from about 50 m²/g to about 200 m²/g and a pore volume of from about 0.5 mL/g to about 1.5 mL/g, and the enzyme(s) is/are selected from list (A), or even more preferably from list (B).

In some embodiments, the immobilized enzyme retains at least 20%, preferably at least 30%; more preferably at least 40%, even more preferably at least 50%, still more preferably at least 60%, yet more preferably at least 70%, further more preferably at least 80%, and most preferably at least 90% of its activity after immobilization compared to the state prior to immobilization. In rare cases, the immobilization may even enhance activity resulting in more than 100% apparent retention. In some embodiments, the immobilized enzyme retains 20%-100%, preferably 30%-100%; more preferably 40%-100%, even more preferably 50%-100%, still more preferably 60%-100%, yet more preferably 70%-100%, further more preferably 80%-100%, and most preferably 90%-100% of its activity after immobilization compared to the state prior to immobilization.

In some embodiments the immobilized enzyme retains at least 20%, preferably at least 30%; more preferably at least 40%, even more preferably at least 50%, still more preferably at least 60%, yet more preferably at least 70%, further more preferably at least 80%, and most preferably at least 90% of its activity after 20 h under continuous flow conditions compared to the state prior to immobilization. In some embodiments the immobilized enzyme retains 20%-100%, preferably 30%-100%; more preferably 40%-100%, even more preferably 50%-100%, still more preferably 60%-100%, yet more preferably 70%-100%, further more preferably 80%-100%, and most preferably 90%-100% of its activity after 20 h under continuous flow conditions compared to the state prior to immobilization.

SEQUENCES

SEQ ID NO: 1 = Thermomyces lanuginosus lipase
MRSSLVLFFV SAWTALASPI RREVSQDLFN QFNLFAQYSA AAYCGKNNDA
PAGTNITCTG NACPEVEKAD ATFLYSFEDS GVGDVTGFLA LDNTNKLIVL
SFRGSRSIEN WIGNLNFDLK EINDICSGCR GHDGFTSSWR SVADTLRQKV
EDAVREHPDY RVVFTGHSLG GALATVAGAD LRGNGYDIDV FSYGAPRVGN
RAFAEFLTVQ TGGTLYRITH TNDIVPRLPP REFGYSHSSP EYWIKSGTLV
PVTRNDIVKI EGIDATGGNN QPNIPDIPAH LWYFGLIGTC L

### Examples

The processes of the present disclosure are further illustrated by the following Examples. These examples should not be viewed in a limiting sense.

### Materials and Methods

### Analytical Methods

Solid fat content (SFC)—Official Methods of the American Oil Chemist's Society, Cd 16b-93, revised 2017.

Iodine value—Official Methods of the American Oil Chemist's Society, Cd 1d-92, reapproved 2017.

Dropping point—Official Methods of the American Oil Chemist's Society, Cc 18-80, reapproved 2017.

Triglycerides in vegetable oils by HPLC (carbon number)—Official Methods of the American Oil Chemist's Society, Ce 5-86, revised 2017.

### Experimental Equipment

1.2 liter glass reaction vessel, unjacketed, 150 mm Schott O-ring, 12 mm drain valve, 12 mm PTFE stem and control knob on bottom for draining and sample removal.

4 hole lid with clamps and o-rings for Duran reaction vessel.

Electrical heating mantel, jacketed.

Temperature controller, J-Kem Scientific—Gemini.

Overhead glass stirrer with Teflon blade and stirrer bearings (teflon).

Temperature prob—K type.

Vacuum pump.

Buchner Filter, 70 mm.

Wattman No. 1 filter paper, 70 mm

2 liter vacuum flask

Hotplate with magnetic stirrer

Magnetic stir bar, 20 mm

### Enzymes

Lipozyme® TL-IM—An immobilized, granulated form of the lipase from Thermomyces lanuginosus having SEQ ID NO:1. The Thermomyces lanuginosus lipase is a 1,3 specific lipase that is immobilized on a silica gel carrier and formed into an immobilized granulate. Lipozyme® TL-IM is commercially available from Novozymes. The label for Lipozyme® TL-IM indicates a maximum usage temperature of 70° C.

Test enzyme I— Lipozyme® TL 100 L, a Thermomyces lanuginosus lipase having SEQ ID NO: 1, was purchased from Novozymes and immobilized on a carrier, as described in Example 2.

Test enzyme II—Lipozyme® TL 100 L, a Thermomyces lanuginosus lipase having SEQ ID NO: 1, was purchased from Novozymes and immobilized on a carrier, as described in Example 3.

### Oils

The oils used in the examples were an 85:15 or 65:35 blend of fractionated palm stearin oil and palm kernel oil. Table 1 below sets forth the properties of the 85:15 blend of fractionated palm stearin oil/palm kernel oil before enzymatic treatment (unreacted), and the target profile after enzymatic treatment (reacted). The profile of fractionated palm stearin oil, palm kernel oil, and the 65:35 blend of fractionated palm stearin oil/palm kernel oil prior to enzymatic treatment is set forth in Table 2 and Table 2a.

TABLE 1
Profile of 85:15 blend of fractionated palm stearin
oil/palm kernel oil used for trials 1 through 11.

| SFC | Unreacted | Reacted |
|---|---|---|
| 10.0° C. | 74-82 | 74-80 |
| 21.1° C. | | 52-58 |
| 26.7° C. | | 39-44 |
| 33.3° C. | | 24-28 |
| 40.0° C. | 20-30 | 14 max |
| Dropping point ° C. | 51-54 | 46° C. max |
| Iodine value (%) | 30-34 | 30-34 |
| C12:0 | 5.5-8.5 | |
| Carbon number 50 | — | 30% max |

TABLE 2
Profile of fractionated palm stearin oil, palm kernel
oil, and 65:35 blend of fractionated palm stearin
oil/palm kernel oil used for trials 12 through 25.

| | Fractionated | | Unreacted | Reacted |
|---|---|---|---|---|
| | palm stearin | Palm kernel | blend | blend |
| | oil | oil (PKO) | 65:35 | 65:35 |
| SFC | | | | |
| 10.0° C. | 79.5 | 65.4 | 69.7 | 68-76 |
| 21.1° C. | 64.3 | 38.5 | 41.5 | |
| 26.7° C. | 52.9 | 8.9 | 30.7 | |
| 33.3° C. | 39.1 | 0.5 | 20.5 | |
| 40.0° C. | 28.1 | 0.0 | 13.5 | 4.0% max |
| Dropping | 55.1 | 28.5 | 51.3 | 42° C. max |
| point ° C. | | | | |
| Iodine | 34.1 | 17.8 | 28.4 | |
| value (%) | | | | |
| Carbon | | | | |
| number (%) | | | | |
| C24 | | | | |
| C26 | | 0.63 | 0.25 | |
| C28 | | 0.64 | 0.28 | |
| C30 | | 1.26 | 0.49 | |
| C32 | 0.07 | 6.16 | 2.27 | |
| C34 | 1.69 | 8.14 | 2.90 | |
| C36 | 2.56 | 20.62 | 7.33 | |
| C38 | 1.34 | 15.92 | 5.74 | |
| C40 | 0.10 | 9.71 | 3.51 | |
| C42 | 0.13 | 9.20 | 3.36 | |
| C44 | 0.24 | 6.82 | 2.60 | |
| C46 | 2.09 | 5.62 | 3.44 | |
| C48 | 25.81 | 6.38 | 19.81 | |
| C50 | 36.25 | 2.77 | 25.59 | |
| C52 | 23.26 | 2.83 | 16.80 | |
| C54 | 6.08 | 3.08 | 5.13 | |
| >C54 | 0.39 | 0.21 | 0.49 | |
| CN44 + 46 | 2.33 | 12.44 | 6.04 | 27% min |

TABLE 2a
Profile of fractionated palm stearin oil, palm kernel
oil, and 65:35 blend of fractionated palm oil/palm
kernel oil used for trials 26 through 36.

| | | | Unreacted | Reacted |
|---|---|---|---|---|
| | Fractionated | Palm kernel | blend | blend |
| | palm oil | oil (PKO) | 65:35 | 65:35 |
| SFC | | | | |
| 10.0° C. | 81.0 | 69.0 | 73.0 | 68-76 |
| 21.1° C. | 65.5 | 39.7 | 44.8 | |
| 26.7° C. | 55.7 | 13.5 | 33.8 | |
| 33.3° C. | 40.7 | 0.0 | 21.8 | |
| 40.0° C. | 29.1 | 0.0 | 13.7 | 4.0% max |
| Dropping | 54.7 | 31.8 | 51.7 | 42° C. max |
| point ° C. | | | | |
| Carbon | | | | |
| number (%) | | | | |
| <C24 | 0.03 | | 0.03 | |
| C24 | 0.02 | 0.04 | 0.03 | |
| C26 | 0.03 | 0.19 | 0.08 | |
| C28 | 0.04 | 0.33 | 0.11 | |
| C30 | | 1.22 | 0.38 | |
| C32 | | 6.35 | 2.00 | |
| C34 | | 8.55 | 2.69 | |
| C36 | 0.48 | 21.48 | 6.75 | |
| C38 | 1.16 | 15.99 | 5.04 | |
| C40 | 0.73 | 9.58 | 3.05 | |
| C42 | 0.08 | 9.07 | 2.84 | |
| C44 | 0.10 | 6.59 | 2.15 | |
| C46 | 1.93 | 5.38 | 3.08 | |
| C48 | 26.72 | 6.21 | 20.20 | |
| C50 | 38.39 | 2.82 | 28.50 | |
| C52 | 23.32 | 2.92 | 17.54 | |
| C54 | 5.81 | 3.05 | 5.12 | |
| >C54 | 0.65 | 0.23 | 0.42 | |
| CN44 + 46 | 2.03 | 11.97 | 5.23 | 27 min |

### Example 1: Batch Enzymatic Interesterification

In this example, an 85:15 or 65:35 blend of fractionated palm oil and palm kernel oil was subjected to batch enzymatic interesterification under varying reaction conditions using either Lipozyme® TL-IM, or the immobilized Thermomyces lanuginosus lipase prepared as described in Example 2 (“test enzyme I”) and Example 3 (“test enzyme II”).

### Trial 1: 0.12 wt % Dosage of Lipozyme® TL-IM at 70° C.; 85:15 Blend

A 1700 gram sample of the 85:15 blend of fractionated palm oil/palm kernel oil was added to a 2 liter reaction vessel. The temperature was set to 70° C. and heated under stirring to the reaction temperature. A 0.12 wt % dosage (by weight of the oil) of Lipozyme® TL-IM was added to the reaction vessel through an open port. The oil was mixed with the enzyme at 70° C. for 8 hours. While the reaction was occurring, a 200 to 250 mL sample of the oil was pulled from the bottom of the vessel and the reacted product was filtered at the reaction temperature via a vacuum Buchner funnel into a vacuum flask with a stir bar. The vacuum flask was on a hot plate with enough heat to maintain the temperature and with stirring. The oil samples were analyzed using the techniques described above. Unless otherwise indicated, dosages disclosed in the examples are amount of the immobilized enzyme by weight of the oil.

Reaction completion is achieved when the physical characteristics of the oil does not change with increasing reaction time. Reaction completion was based on complete randomization of the fatty acids on the triacylglycerols, and was determined by measuring SFC, dropping point, and carbon number of the triglycerides in the oil.

The profile of the oil produced by Trial 1 is set forth below in Table 3.

TABLE 3
Lipozyme ® TL-IM, 0.12 wt % @ 70° C. (85:15 blend)

Time (hours)

| | 0 | 2 | 4 | 6 | 8 |
|---|---|---|---|---|---|
| SFC | | | | | |
| 10.0° C. | 73.9 | | | | 73.9 |
| 21.1° C. | 54.2 | | | | 53.4 |
| 26.7° C. | 42.5 | | | | 41.5 |
| 33.3° C. | 29.4 | | | | 28.0 |
| 40.0° C. | 19.3 | | | | 17.0 |
| Dropping | 54.1 | 53.8 | 53.7 | 53.5 | 53.2 |
| point (° C.) | | | | | |
| Carbon | | | | | |
| number (%) | | | | | |
| C24 | 0.07 | | | | 0.32 |
| C26 | 0.15 | | | | 0.19 |
| C28 | 0.15 | | | | 0.12 |
| C30 | 0.14 | | | | 0.28 |
| C32 | 1.07 | | | | 0.72 |
| C34 | 1.38 | | | | 1.03 |
| C36 | 3.68 | | | | 3.09 |
| C38 | 3.03 | | | | 2.56 |
| C40 | 1.85 | | | | 2.26 |
| C42 | 1.65 | | | | 2.32 |
| C44 | 1.25 | | | | 2.97 |
| C46 | 2.63 | | | | 4.77 |
| C48 | 23.92 | | | | 22.86 |
| C50 | 33.35 | | | | 32.01 |
| C52 | 20.11 | | | | 19.29 |
| C54 | 5.25 | | | | 4.76 |
| >C54 | 0.33 | | | | 0.45 |

The reaction did not reach completion after 8 hours using a low dosage (0.12 wt % o) of Lipozyme® TL-IM.

### Trial 2: 0.24 wt % Dosage of Lipozyme® TL-IM at 70° C.; 85:15 Blend

Trial 2 was performed using the same procedure as described for Trial 1, except the dose of Lipozyme® TL-TM was 0.24 wt % o, and the reaction was allowed to proceed for 10 hours.

The profile of the oil produced by Trial 2 is set forth below in Table 4.

TABLE 4
Lipozyme ® TL-IM, 0.24 wt % @ 70° C. (85:15 blend)

| | Time (hours | | | | | |
|---|---|---|---|---|---|---|
| | 0 | 2 | 4 | 6 | 8 | 10 |
| SFC | | | | | | |
| 10.0° C. | 73.9 | | | | | 75.2 |
| 21.1° C. | 54.2 | | | | | 53.5 |
| 26.7° C. | 42.5 | | | | | 41.0 |
| 33.3° C. | 29.4 | | | | | 26.6 |
| 40.0° C. | 19.3 | | | | | 15.0 |
| Dropping point (° C.) | 54.1 | 53.4 | 52.4 | 51.8 | 47.6 | 47.3 |
| Carbon number (%) | | | | | | |
| C24 | 0.07 | | | | | |
| C26 | 0.15 | | | | | 0.10 |
| C28 | 0.15 | | | | | 0.10 |
| C30 | 0.14 | | | | | 0.51 |
| C32 | 1.07 | | | | | 0.51 |
| C34 | 1.38 | | | | | 0.71 |
| C36 | 3.68 | | | | | 2.26 |
| C38 | 3.03 | | | | | 2.07 |
| C40 | 1.85 | | | | | 2.77 |
| C42 | 1.65 | | | | | 3.08 |
| C44 | 1.25 | | | | | 5.23 |
| C46 | 2.63 | | | | | 7.62 |
| C48 | 23.92 | | | | | 21.65 |
| C50 | 33.35 | | | | | 30.41 |
| C52 | 20.11 | | | | | 18.17 |
| C54 | 5.25 | | | | | 4.41 |
| >C54 | 0.33 | | | | | 0.38 |

The reaction did not reach completion after 10 hours using a dosage of 0.24 wt % of Lipozyme® TL-JM at 70° C.

### Trial 3: 0.48 wt % Dosage of Lipozyme® TL-IM at 70° C.; 85:15 Blend

Trial 3 was performed using the same procedure as described for Trial 1, except the dose of Lipozyme® TL-TM was 0.48 wt % o, and the reaction was allowed to proceed for 10 hours.

The profile of the oil produced by Trial 3 is set forth below in Table 5.

TABLE 5
Lipozyme TL-IM, 0.48 wt % @ 70° C. (85:15 blend)

| | Time (hours) | | | | | |
|---|---|---|---|---|---|---|
| | 0 | 2 | 4 | 6 | 8 | 10 |
| SFC | | | | | | |
| 10.0° C. | 73.9 | | 75.2 | 75.8 | 77.1 | 76.7 |
| 21.1° C. | 54.2 | | 53.3 | 53.1 | 53.8 | 53.6 |
| 26.7° C. | 42.5 | | 40.6 | 40.5 | 40.9 | 40.7 |
| 33.3° C. | 29.4 | | 26.6 | 26.1 | 26.1 | 25.9 |
| 40.0° C. | 19.3 | | 14.4 | 13.4 | 11.5 | 11.6 |
| Dropping point (° C.) | 54.1 | 52.2 | 47.2 | 47.0 | 46.8 | 46.4 |
| Carbon number (%) | | | | | | |
| C24 | 0.07 | | | | | |
| C26 | 0.15 | | 0.10 | 0.15 | 0.14 | 0.15 |
| C28 | 0.15 | | 0.10 | 0.10 | 0.13 | 0.11 |
| C30 | 0.14 | | 0.51 | 0.49 | 0.60 | 0.64 |
| C32 | 1.07 | | 0.51 | 0.47 | 0.30 | 0.17 |
| C34 | 1.38 | | 0.71 | 0.66 | 0.45 | 0.35 |
| C36 | 3.68 | | 2.26 | 1.94 | 1.30 | 1.09 |
| C38 | 3.03 | | 2.07 | 1.63 | 1.49 | 1.34 |
| C40 | 1.85 | | 2.77 | 2.62 | 2.89 | 2.84 |
| C42 | 1.65 | | 3.08 | 3.09 | 3.57 | 3.69 |
| C44 | 1.25 | | 5.23 | 5.33 | 7.02 | 7.59 |
| C46 | 2.63 | | 7.62 | 7.77 | 9.93 | 10.62 |
| C48 | 23.92 | | 21.65 | 21.85 | 21.00 | 20.87 |
| C50 | 33.35 | | 30.41 | 30.76 | 29.47 | 29.22 |
| C52 | 20.11 | | 18.17 | 18.38 | 17.30 | 17.16 |
| C54 | 5.25 | | 4.41 | 4.44 | 4.00 | 3.80 |
| >C54 | 0.33 | | 0.38 | 0.32 | 0.40 | 0.36 |

The reaction did not reach completion after 10 hours using a dosage of 0.48 wt % of Lipozyme® TL-IM at 70° C.

### Trial 4: 0.72 wt % Dosage of Lipozyme® TL-IM at 70° C.; 85:15 Blend

Trial 4 was performed using the same procedure as described for Trial 1, except the dose of Lipozyme® TL-IM was 0.72 wt %, and the reaction was allowed to proceed for 10 hours. Once the immobilized enzyme was removed from the oil, it was kept in a sealed container for reuse.

The profile of the oil produced by Trial 4 is set forth below in Table 6.

TABLE 6
Lipozyme ® TL-IM, 0.72 wt % @ 70° C. (85:15 blend)

| | Time (hours) | | | | | |
|---|---|---|---|---|---|---|
| | 0 | 2 | 4 | 6 | 8 | 10 |
| SFC | | | | | | |
| 10.0° C. | 73.9 | 74.9 | 76.1 | 76.2 | 76.9 | 77.1 |
| 21.1° C. | 54.2 | 52.7 | 52.7 | 53.1 | 53.2 | 53.9 |
| 26.7° C. | 42.5 | 40.0 | 40.0 | 40.1 | 39.8 | 40.7 |
| 33.3° C. | 29.4 | 26.3 | 25.5 | 25.0 | 24.8 | 25.3 |
| 40.0° C. | 19.3 | 13.6 | 11.0 | 10.9 | 10.4 | 10.9 |
| Dropping point (° C.) | 54.1 | 47.2 | 46.6 | 46.2 | 46.0 | 45.7 |
| Carbon number (%) | | | | | | |
| C24 | 0.07 | | | | | |
| C26 | 0.15 | 0.19 | 0.12 | 0.15 | 0.12 | 0.20 |
| C28 | 0.15 | 0.11 | 0.13 | 0.13 | 0.12 | 0.18 |
| C30 | 0.14 | 0.57 | 0.62 | 0.74 | 0.72 | 1.18 |
| C32 | 1.07 | 0.70 | 0.43 | 0.37 | 0.68 | 0.35 |
| C34 | 1.38 | 0.82 | 0.63 | 0.37 | 0.27 | 0.35 |
| C36 | 3.68 | 2.27 | 1.54 | 1.23 | 0.90 | 1.04 |
| C38 | 3.03 | 2.21 | 1.61 | 1.43 | 1.25 | 1.21 |
| C40 | 1.85 | 2.52 | 2.75 | 2.84 | 2.84 | 2.79 |
| C42 | 1.65 | 2.87 | 3.37 | 3.62 | 3.76 | 3.76 |
| C44 | 1.25 | 4.67 | 6.39 | 7.47 | 8.13 | 8.54 |
| C46 | 2.63 | 6.96 | 9.13 | 10.49 | 11.33 | 11.86 |
| C48 | 23.92 | 21.92 | 21.25 | 20.77 | 20.64 | 20.20 |
| C50 | 33.35 | 30.86 | 29.89 | 29.20 | 28.42 | 28.07 |
| C52 | 20.11 | 18.38 | 17.64 | 17.16 | 16.78 | 16.36 |
| C54 | 5.25 | 4.51 | 4.09 | 3.74 | 3.67 | 3.54 |
| >C54 | 0.33 | 0.45 | 0.41 | 0.30 | 0.38 | 0.37 |

The reaction reached completion after 8 hours using a dosage of 0.72 wt/wt % of Lipozyme® TL-TM at 70° C.

### Trial 5: 0.72 wt % Dosage of Lipozyme® TL-IM at 70° C.; 85:15 Blend; Enzyme Reuse

Trial 5 was performed using the same procedure as described for Trial 4, except the enzyme was 0.72 wt % of previously used Lipozyme® TL-IM from Trial 4.

The profile of the oil produced by Trial 5 is set forth below in Table 7.

TABLE 7
Lipozyme ® TL-IM, 0.72 wt % @ 70°
C. (85:15 blend), enzyme reuse

| | Time (hours) | | | | | |
|---|---|---|---|---|---|---|
| | 0 | 2 | 4 | 6 | 8 | 10 |
| SFC | | | | | | |
| 10.0° C. | 73.9 | 75.5 | 74.8 | 76.5 | 76.6 | 74.4 |
| 21.1° C. | 54.2 | 54.1 | 54.2 | 54.4 | 54.2 | 53.2 |
| 26.7° C. | 42.5 | 41.7 | 41.5 | 41.3 | 41.0 | 40.9 |
| 33.3° C. | 29.4 | 27.8 | 27.3 | 26.4 | 26.7 | 25.8 |
| 40.0° C. | 19.3 | 16.1 | 14.3 | 13.4 | 13.2 | 12.5 |
| Dropping point (° C.) | 54.1 | 52.4 | 47.5 | 47.1 | 46.8 | 46.7 |
| Carbon number (%) | | | | | | |
| C24 | 0.07 | | | | | |
| C26 | 0.15 | 0.27 | 0.26 | 0.19 | 0.12 | 0.11 |
| C28 | 0.15 | 0.22 | 0.17 | 0.09 | 0.13 | 0.22 |
| C30 | 0.14 | 0.94 | 0.53 | 0.60 | 0.56 | 0.72 |
| C32 | 1.07 | 0.95 | 0.65 | 0.36 | 0.26 | 0.56 |
| C34 | 1.38 | 1.30 | 0.96 | 0.65 | 0.47 | 0.56 |
| C36 | 3.68 | 3.07 | 2.49 | 1.74 | 1.42 | 1.28 |
| C38 | 3.03 | 2.43 | 2.09 | 1.90 | 1.50 | 1.45 |
| C40 | 1.85 | 2.38 | 2.70 | 2.82 | 2.82 | 3.06 |
| C42 | 1.65 | 2.52 | 3.05 | 3.34 | 3.54 | 3.78 |
| C44 | 1.25 | 3.58 | 4.97 | 6.09 | 6.86 | 7.54 |
| C46 | 2.63 | 5.51 | 7.26 | 8.71 | 9.67 | 10.43 |
| C48 | 23.92 | 22.17 | 21.59 | 21.33 | 21.14 | 20.89 |
| C50 | 33.35 | 31.07 | 30.32 | 29.90 | 29.52 | 28.75 |
| C52 | 20.11 | 18.53 | 18.04 | 17.79 | 17.45 | 16.58 |
| C54 | 5.25 | 4.78 | 4.57 | 4.24 | 4.17 | 3.63 |
| >C54 | 0.33 | 0.29 | 0.34 | 0.24 | 0.37 | 0.43 |

The reaction did not reach completion after 10 hours using a dosage of 0.72 wt % of reused Lipozyme® TL-IM at 70° C.

### Trial 6: 0.72 wt % Dosage of Test Enzyme T at 70° C.; 85:15 Blend

Trial 6 was performed using the same procedure as described for Trial 1, except the Test Enzyme I prepared as described in Example 2 was used at a dosage of 0.72 wt % o. Once the immobilized enzyme was removed from the oil, it was kept in a sealed container for reuse.

The profile of the oil produced by Trial 6 is set forth below in Table 8.

TABLE 8
Test Enzyme I, 0.72 wt % @ 70° C. (85:15 blend)

Time (hours)

| | 0 | 2 | 4 | 6 | 8 |
|---|---|---|---|---|---|
| SFC | | | | | |
| 10.0° C. | 73.9 | 77.1 | 77.5 | 77.8 | 77.8 |
| 21.1° C. | 54.2 | 54.0 | 55.4 | 55.8 | 56.9 |
| 26.7° C. | 42.5 | 41.0 | 42.0 | 42.6 | 43.4 |
| 33.3° C. | 29.4 | 24.8 | 25.7 | 26.1 | 26.7 |
| 40.0° C. | 19.3 | 10.0 | 10.1 | 10.2 | 10.8 |
| Dropping | 54.1 | 45.6 | 45.5 | 45.5 | 45.5 |
| point (° C.) | | | | | |
| Carbon | | | | | |
| number (%) | | | | | |
| C24 | 0.07 | | | | |
| C26 | 0.15 | 0.10 | 0.16 | 0.10 | 0.11 |
| C28 | 0.15 | 0.13 | 0.07 | 0.12 | 0.10 |
| C30 | 0.14 | 0.68 | 0.66 | 0.68 | 0.73 |
| C32 | 1.07 | 0.69 | 0.67 | 0.70 | 0.74 |
| C34 | 1.38 | 1.01 | 0.25 | 0.69 | 0.98 |
| C36 | 3.68 | 0.57 | 0.44 | 0.43 | 0.42 |
| C38 | 3.03 | 0.86 | 0.81 | 0.79 | 0.80 |
| C40 | 1.85 | 2.76 | 2.62 | 2.58 | 2.63 |
| C42 | 1.65 | 3.93 | 3.82 | 3.80 | 3.81 |
| C44 | 1.25 | 9.37 | 9.76 | 9.76 | 9.80 |
| C46 | 2.63 | 12.94 | 13.54 | 13.52 | 13.49 |
| C48 | 23.92 | 19.99 | 20.14 | 20.28 | 20.42 |
| C50 | 33.35 | 27.72 | 27.54 | 27.06 | 26.70 |
| C52 | 20.11 | 15.82 | 15.98 | 15.90 | 15.67 |
| C54 | 5.25 | 3.24 | 3.30 | 3.33 | 3.28 |
| >C54 | 0.33 | 0.19 | 0.25 | 0.25 | 0.32 |

The reaction reached completion after 2 hours using a dosage of 0.72 wt % of the Test Enzyme I at 70° C.

### Trial 7: 0.72 wt % Dosage of Test Enzyme I at 70° C.; 85:15 Blend, Enzyme Reuse

Trial 7 was performed using the same procedure as described for Trial 6, except the enzyme was 0.72 wt % of previously used Test Enzyme from Trial 6, and the reaction was allowed to proceed for 4 hours. Once the immobilized enzyme was removed from the oil, it was kept in a sealed container for reuse.

The profile of the oil produced by Trial 7 is set forth below in Table 9.

TABLE 9
Test Enzyme I, 0.72 wt % @ 70° C. (85:15 blend), enzyme reuse

| | Time (hours) | | |
|---|---|---|---|
| | 0 | 2 | 4 |
| SFC | | | |
| 10.0° C. | 73.9 | 75.6 | 76.1 |
| 21.1° C. | 54.2 | 53.0 | 53.0 |
| 26.7° C. | 42.5 | 39.9 | 39.9 |
| 33.3° C. | 29.4 | 24.7 | 24.3 |
| 40.0° C. | 19.3 | 10.1 | 9.6 |
| Dropping | 54.1 | 45.6 | 45.5 |
| point (° C.) | | | |
| Carbon | | | |
| number (%) | | | |
| C24 | 0.07 | | |
| C26 | 0.15 | 0.12 | 0.08 |
| C28 | 0.15 | 0.11 | 0.09 |
| C30 | 0.14 | 0.71 | 0.72 |
| C32 | 1.07 | 0.74 | 0.71 |
| C34 | 1.38 | 1.08 | 0.96 |
| C36 | 3.68 | 0.73 | 0.54 |
| C38 | 3.03 | 1.03 | 0.82 |
| C40 | 1.85 | 2.92 | 2.72 |
| C42 | 1.65 | 3.90 | 3.85 |
| C44 | 1.25 | 8.55 | 9.21 |
| C46 | 2.63 | 11.89 | 12.82 |
| C48 | 23.92 | 20.10 | 19.93 |
| C50 | 33.35 | 28.28 | 27.97 |
| C52 | 20.11 | 16.19 | 16.09 |
| C54 | 5.25 | 3.34 | 3.30 |
| >C54 | 0.33 | 0.32 | 0.20 |

The reaction reached completion after 2 hours using a dosage of 0.72 wt % of once reused Test Enzyme I (effective dosage 0.36 wt %) at 70° C.

### Trail 8: 0.72 wt % Dosage of Test Enzyme I at 70° C.; 85:15 Blend, Enzyme Twice Reused

Trial 8 was performed using the same procedure as described for Trial 7, except the enzyme was 0.72 wt % of previously used Test Enzyme I from Trial 7 (previously used in Trials 6 and 7).

The profile of the oil produced by Trial 8 is set forth below in Table 10.

TABLE 10
Test Enzyme I, 0.72 wt % @ 70° C.
(85:15 blend), enzyme twice reused

| | Time (hours) | | |
|---|---|---|---|
| | 0 | 2 | 4 |
| SFC | | | |
| 10.0° C. | 73.9 | 75.7 | 76.2 |
| 21.1° C. | 54.2 | 53.2 | 52.7 |
| 26.7° C. | 42.5 | 40.6 | 40.0 |
| 33.3° C. | 29.4 | 25.9 | 24.5 |
| 40.0° C. | 19.3 | 12.6 | 10.6 |
| Dropping | 54.1 | 46.8 | 45.5 |
| point (° C.) | | | |
| Carbon | | | |
| number (%) | | | |
| C24 | 0.07 | | |
| C26 | 0.15 | 0.12 | 0.08 |
| C28 | 0.15 | 0.10 | 0.10 |
| C30 | 0.14 | 0.56 | 0.61 |
| C32 | 1.07 | 0.41 | 0.67 |
| C34 | 1.38 | 0.37 | 1.01 |
| C36 | 3.68 | 1.54 | 0.78 |
| C38 | 3.03 | 1.46 | 1.05 |
| C40 | 1.85 | 3.08 | 2.94 |
| C42 | 1.65 | 3.72 | 3.89 |
| C44 | 1.25 | 6.69 | 8.26 |
| C46 | 2.63 | 9.51 | 11.52 |
| C48 | 23.92 | 20.63 | 20.10 |
| C50 | 33.35 | 29.74 | 28.44 |
| C52 | 20.11 | 17.62 | 16.49 |
| C54 | 5.25 | 4.01 | 3.48 |
| >C54 | 0.33 | 0.46 | 0.56 |

The reaction reached completion after 4 hours using a dosage of 0.72 wt % of a twice reused Test Enzyme I (effective dosage 0.24 wt %) at 70° C.

### Trial 9: 0.48 wt % Dosage of Lipozyme® TL-IM at 80° C.; 85:15 Blend

Trial 9 was performed using the same procedure as described for Trial 3, except the temperature was 80° C.

The profile of the oil produced by Trial 9 is set forth below in Table 11.

TABLE 11
Lipozyme ® TL-IM, 0.48 wt % @ 80° C. (85:15 blend)

| | Time (hours) | | | | | |
|---|---|---|---|---|---|---|
| | 0 | 2 | 4 | 6 | 8 | 10 |
| SFC | | | | | | |
| 10.0° C. | 73.9 | 75.5 | 76.2 | 76.8 | 76.9 | 77.4 |
| 21.1° C. | 54.2 | 53.5 | 53.6 | 53.6 | 53.3 | 54.1 |
| 26.7° C. | 42.5 | 41.0 | 40.7 | 40.8 | 40.2 | 40.7 |
| 33.3° C. | 29.4 | 27.0 | 26.4 | 35.9 | 25.1 | 25.4 |
| 40.0° C. | 19.3 | 14.8 | 12.8 | 11.9 | 11.7 | 11.7 |
| Dropping point (° C.) | 54.1 | 47.2 | 47 | 46.4 | 46.3 | 46 |
| Carbon number (%) | | | | | | |
| C24 | 0.07 | | | | | |
| C26 | 0.15 | 0.12 | 0.11 | 0.11 | 0.10 | 0.10 |
| C28 | 0.15 | 0.14 | 0.14 | 0.14 | 0.14 | 0.15 |
| C30 | 0.14 | 0.50 | 0.57 | 0.60 | 0.61 | 0.63 |
| C32 | 1.07 | 0.91 | 0.83 | 0.78 | 0.74 | 0.73 |
| C34 | 1.38 | 0.70 | 0.53 | 0.45 | 0.40 | 0.35 |
| C36 | 3.68 | 2.21 | 1.67 | 1.31 | 1.12 | 0.98 |
| C38 | 3.03 | 2.13 | 1.77 | 1.53 | 1.39 | 1.29 |
| C40 | 1.85 | 2.51 | 2.72 | 2.80 | 2.84 | 2.83 |
| C42 | 1.65 | 2.81 | 3.25 | 3.49 | 3.63 | 3.68 |
| C44 | 1.25 | 4.56 | 5.92 | 6.83 | 7.38 | 7.76 |
| C46 | 2.63 | 6.78 | 8.48 | 9.63 | 10.38 | 10.81 |
| C48 | 23.92 | 21.81 | 21.19 | 20.75 | 20.42 | 20.43 |
| C50 | 33.35 | 31.12 | 30.07 | 29.48 | 29.12 | 28.82 |
| C52 | 20.11 | 18.71 | 18.08 | 17.58 | 17.42 | 17.07 |
| C54 | 5.25 | 4.68 | 4.38 | 4.17 | 4.05 | 3.92 |
| >C54 | 0.33 | 0.32 | 0.30 | 0.36 | 0.27 | 0.45 |

The reaction did not reach completion after 10 hours using a dosage of 0.48 wt % of Lipozyme® TL-IM at 80° C.

### Trial 10: 0.48 wt % Dosage of Lipozyme® TL-IM at 85° C.; 85:15 Blend

Trial 10 was performed using the same procedure as described for Trial 9, except the temperature was 85° C.

The profile of the oil produced by Trial 10 is set forth below in Table 12.

TABLE 12
Lipozyme ® TL-IM, 0.48 wt % @ 85° C. (85:15 blend)

| | Time (hours) | | | | | |
|---|---|---|---|---|---|---|
| | 0 | 2 | 4 | 6 | 8 | 10 |
| SFC | | | | | | |
| 10.0° C. | 73.9 | 74.7 | 75.3 | 76.0 | 76.1 | 76.2 |
| 21.1° C. | 54.2 | 52.8 | 53.0 | 52.9 | 53.4 | 52.8 |
| 26.7° C. | 42.5 | 40.4 | 40.3 | 40.2 | 40.7 | 40.1 |
| 33.3° C. | 29.4 | 26.9 | 25.9 | 25.5 | 25.6 | 24.9 |
| 40.0° C. | 19.3 | 14.4 | 12.4 | 12.1 | 11.0 | 10.9 |
| Dropping point (° C.) | 54.1 | 47.3 | 46.6 | 46.6 | 46 | 46.2 |
| Carbon number (%) | | | | | | |
| C24 | 0.07 | | | | | |
| C26 | 0.15 | 0.10 | 0.12 | 0.10 | 0.13 | 0.09 |
| C28 | 0.15 | 0.14 | 0.17 | 0.13 | 0.15 | 0.14 |
| C30 | 0.14 | 0.52 | 0.62 | 0.59 | 0.54 | 0.62 |
| C32 | 1.07 | 0.92 | 0.97 | 0.77 | 0.93 | 0.73 |
| C34 | 1.38 | 0.68 | 0.58 | 0.41 | 0.48 | 0.35 |
| C36 | 3.68 | 2.19 | 1.66 | 1.32 | 1.22 | 1.01 |
| C38 | 3.03 | 2.12 | 2.29 | 1.52 | 1.44 | 1.30 |
| C40 | 1.85 | 2.56 | 2.72 | 2.80 | 2.85 | 2.85 |
| C42 | 1.65 | 2.91 | 3.29 | 3.49 | 3.62 | 3.70 |
| C44 | 1.25 | 4.76 | 6.05 | 6.84 | 7.39 | 7.73 |
| C46 | 2.63 | 7.03 | 8.67 | 9.67 | 10.34 | 10.81 |
| C48 | 23.92 | 21.58 | 20.69 | 20.57 | 20.47 | 20.30 |
| C50 | 33.35 | 30.80 | 29.64 | 29.32 | 28.97 | 28.80 |
| C52 | 20.11 | 18.69 | 17.91 | 17.56 | 17.22 | 17.21 |
| C54 | 5.25 | 4.69 | 4.32 | 4.16 | 3.99 | 4.08 |
| >C54 | 0.33 | 0.31 | 0.30 | 0.74 | 0.27 | 0.29 |

The reaction did not reach completion after 10 hours using a dosage of 0.48 wt % of Lipozyme® TL-IM at 85° C. The sample taken at 8 hours reached all parameters, but then failed at 10 hours due to a higher MDP.

### Trial 11: 0.48 wt % Dosage of Lipozyme® TL-IM at 90° C.; 85:15 Blend

Trial 11 was performed using the same procedure as described for Trial 9, except the temperature was 90° C.

The profile of the oil produced by Trial 11 is set forth below in Table 13.

TABLE 13
Lipozyme ® TL-IM, 0.48 wt % @ 90° C. (85:15 blend)

| | Time (hours) | | | | | |
|---|---|---|---|---|---|---|
| | 0 | 2 | 4 | 6 | 8 | 10 |
| SFC | | | | | | |
| 10.0° C. | 73.9 | 74.2 | 75.2 | 76.6 | 76.2 | 75.9 |
| 21.1° C. | 54.2 | 51.6 | 52.1 | 53.6 | 53.0 | 53.1 |
| 26.7° C. | 42.5 | 39.6 | 39.9 | 41.1 | 40.4 | 40.3 |
| 33.3° C. | 29.4 | 26.1 | 25.5 | 26.4 | 25.4 | 25.1 |
| 40.0° C. | 19.3 | 14.9 | 12.8 | 12.3 | 11.6 | 11.1 |
| Dropping point (° C.) | 54.1 | 46.7 | 46.4 | 46.2 | 46 | 45.8 |
| Carbon number (%) | | | | | | |
| C24 | 0.07 | | | | | |
| C26 | 0.15 | 0.21 | 0.15 | 0.18 | 0.10 | 0.16 |
| C28 | 0.15 | 0.20 | 0.21 | 0.19 | 0.14 | 0.19 |
| C32 | 1.07 | 1.15 | 1.04 | 0.94 | 0.74 | 0.93 |
| C34 | 1.38 | 0.85 | 0.65 | 0.43 | 1.02 | 0.78 |
| C36 | 3.68 | 2.27 | 1.65 | 1.29 | 1.01 | 0.94 |
| C38 | 3.03 | 2.13 | 1.73 | 1.49 | 1.31 | 1.27 |
| C40 | 1.85 | 2.56 | 2.76 | 2.80 | 2.81 | 2.79 |
| C42 | 1.65 | 2.91 | 3.35 | 3.52 | 3.62 | 3.65 |
| C44 | 1.25 | 4.80 | 6.24 | 7.09 | 7.54 | 7.84 |
| C46 | 2.63 | 7.08 | 8.87 | 9.91 | 10.54 | 10.92 |
| C48 | 23.92 | 21.40 | 20.79 | 20.91 | 20.30 | 20.20 |
| C50 | 33.35 | 30.52 | 29.67 | 29.07 | 28.86 | 28.47 |
| C52 | 20.11 | 18.43 | 17.77 | 17.11 | 17.17 | 16.84 |
| C54 | 5.25 | 4.60 | 4.26 | 3.99 | 3.95 | 3.99 |
| >C54 | 0.33 | 0.31 | 0.29 | 0.47 | 0.27 | 0.29 |

The reaction did not reach completion after 8 hours using a dosage of 0.48 wt % of Lipozyme® TL-IM at 90° C.

Trials 9-11 demonstrate that the reaction may reach completion after a minimum of 8 hours when the reaction temperature was increased above the manufacturer's recommended temperature (i.e., 70° C.).

### Trial 12: 0.72 wt % Dosage of Lipozyme® TL-IM at 80° C.; 65:35 Blend

Trial 12 was performed using the same procedure as described for Trial 4, except a 65:35 blend of fractionated palm stearin oil/palm kernel oil was used and the temperature was 80° C.

The profile of the oil produced by Trial 12 is set forth below in Table 14.

TABLE 14
Lipozyme ® TL-IM, 0.72 wt % @ 80° C. (65:35 blend)

| | Time (hours) | | | | | |
|---|---|---|---|---|---|---|
| | 0 | 2 | 4 | 6 | 8 | 10 |
| SFC | | | | | | |
| 10.0° C. | 69.7 | 67.7 | 68.3 | 69.3 | 69.0 | 70.0 |
| 21.1° C. | 41.5 | 41.2 | 41.8 | 42.4 | 41.5 | 43.6 |
| 26.7° C. | 30.7 | 28.9 | 28.9 | 28.8 | 28.1 | 29.2 |
| 33.3° C. | 20.5 | 16.5 | 15.3 | 14.4 | 13.8 | 13.8 |
| 40.0° C. | 13.5 | 7.9 | 4.4 | 3.7 | 2.2 | 2.1 |
| Dropping point (° C.) | 51.3 | 44 | 42.4 | 41.5 | 40.5 | 40.8 |
| Carbon number (%) | | | | | | |
| C24 | | | | | | |
| C26 | 0.25 | 0.36 | 0.39 | 0.36 | 0.36 | 0.30 |
| C28 | 0.28 | 0.40 | 0.41 | 0.39 | 0.39 | 0.32 |
| C30 | 0.49 | 0.99 | 1.18 | 1.22 | 1.23 | 1.20 |
| C32 | 2.27 | 1.13 | 0.81 | 0.54 | 0.36 | 0.42 |
| C34 | 2.90 | 1.59 | 1.08 | 0.80 | 0.62 | 0.59 |
| C36 | 7.33 | 4.31 | 3.29 | 2.70 | 2.22 | 2.07 |
| C38 | 5.74 | 4.10 | 3.56 | 3.21 | 2.90 | 2.74 |
| C40 | 3.51 | 5.37 | 6.23 | 6.57 | 6.68 | 6.58 |
| C42 | 3.36 | 6.15 | 7.53 | 8.18 | 8.54 | 8.55 |
| C44 | 2.60 | 7.62 | 10.30 | 11.76 | 12.86 | 13.24 |
| C46 | 3.44 | 9.89 | 13.26 | 15.16 | 16.63 | 17.13 |
| C48 | 19.81 | 17.97 | 16.81 | 16.36 | 16.16 | 16.17 |
| C50 | 25.59 | 21.67 | 19.18 | 18.01 | 17.20 | 16.99 |
| C52 | 16.80 | 14.15 | 12.32 | 11.51 | 10.87 | 10.82 |
| C54 | 5.13 | 3.99 | 3.30 | 2.95 | 2.69 | 2.66 |
| >C54 | 0.49 | 0.31 | 0.34 | 0.29 | 0.28 | 0.23 |
| CN44 + 46 | 6.04 | 17.51 | 23.56 | 26.92 | 29.49 | 30.38 |

The reaction reached completion after 8 hours using a dosage of 0.72 wt % of Lipozyme® TL-8M at 80° C.

Trial 13: 0.24 wt % dosage of Test Enzyme I at 80° C.; 65:35 Blend

Trial 13 was performed using the same procedure as described for Trial 12, except a 0.24 wt % dosage of Test Enzyme I was used. Once the immobilized enzyme was removed from the oil, it was kept in a sealed container for reuse.

The profile of the oil produced by Trial 13 is set forth below in Table 15.

TABLE 15
Test Enzyme I, 0.24 wt % @ 80° C. (65:35 blend)

| | Time (hours) | | | | | |
|---|---|---|---|---|---|---|
| | 0 | 2 | 4 | 6 | 8 | 10 |
| SFC | | | | | | |
| 10.0° C. | 69.7 | 69.7 | 70.2 | 70.6 | 71.3 | 71.8 |
| 21.1° C. | 41.5 | 42.3 | 43.1 | 43.4 | 43.6 | 43.8 |
| 26.7° C. | 30.7 | 29.8 | 29.2 | 30.1 | 29.7 | 29.9 |
| 33.3° C. | 20.5 | 17.9 | 16.5 | 15.9 | 15.3 | 15.1 |
| 40.0° C. | 13.5 | 6.0 | 4.5 | 3.6 | 3.8 | 3.4 |
| Dropping point (° C.) | 51.3 | 44.5 | 43.5 | 42.6 | 42.2 | 41.6 |
| Carbon number (%) | | | | | | |
| C24 | | | | | 0.08 | |
| C26 | 0.25 | 0.43 | 0.34 | 0.40 | 0.49 | 0.37 |
| C28 | 0.28 | 0.41 | 0.34 | 0.42 | 0.49 | 0.39 |
| C30 | 0.49 | 0.98 | 0.98 | 1.16 | 1.31 | 1.16 |
| C32 | 2.27 | 1.90 | 0.95 | 0.97 | 0.55 | 0.53 |
| C34 | 2.90 | 1.85 | 1.24 | 1.04 | 0.78 | 0.74 |
| C36 | 7.33 | 4.90 | 3.78 | 3.26 | 2.57 | 2.58 |
| C38 | 5.74 | 4.55 | 3.79 | 3.56 | 3.12 | 3.15 |
| C40 | 3.51 | 5.24 | 5.90 | 6.47 | 6.32 | 6.78 |
| C42 | 3.36 | 5.82 | 6.94 | 7.75 | 7.85 | 8.41 |
| C44 | 2.60 | 6.83 | 9.05 | 10.54 | 11.35 | 12.19 |
| C46 | 3.44 | 8.82 | 11.72 | 13.45 | 14.63 | 15.54 |
| C48 | 19.81 | 17.90 | 17.29 | 16.68 | 16.62 | 16.20 |
| C50 | 25.59 | 21.84 | 20.43 | 18.90 | 18.47 | 17.67 |
| C52 | 16.80 | 14.22 | 13.35 | 12.02 | 11.89 | 11.12 |
| C54 | 5.13 | 4.06 | 3.65 | 3.16 | 3.06 | 2.78 |
| >C54 | 0.49 | 0.26 | 0.23 | 0.22 | 0.23 | 0.38 |
| CN44 + 46 | 6.04 | 15.64 | 20.77 | 24.00 | 25.98 | 27.73 |

The reaction reached completion after 10 hours using a very low dosage of 00.24 wt % of the Test Enzyme at 80° C.

### Trial 14: 0.24 wt % Dosage of Test Enzyme I at 80° C.; 65:35 Blend, Enzyme Reused

Trial 14 was performed using the same procedure as described for Trial 13, except the enzyme was a 0.24 wt % dosage of the previously used Test Enzyme I from Trial 13. Once the immobilized enzyme was removed from the oil, it was kept in a sealed container for reuse.

The profile of the oil produced by Trial 14 is set forth below in Table 16.

TABLE 16
Test Enzyme I, 0.24 wt % @ 80° C. (65:35 blend), enzyme reused

| | Time (hours) | | | | | |
|---|---|---|---|---|---|---|
| | 0 | 2 | 4 | 6 | 8 | 10 |
| SFC | | | | | | |
| 10.0° C. | 69.7 | 68.6 | 69.6 | 69.2 | 69.0 | 69.4 |
| 21.1° C. | 41.5 | 41.3 | 41.2 | 41.2 | 41.6 | 42.0 |
| 26.7° C. | 30.7 | 29.1 | 28.5 | 28.8 | 28.9 | 28.5 |
| 33.3° C. | 20.5 | 18.6 | 17.7 | 16.9 | 16.5 | 15.8 |
| 40.0° C. | 13.5 | 10.1 | 9.0 | 7.7 | 6.4 | 5.8 |
| Dropping point (° C.) | 51.3 | 45.5 | 44.5 | 44 | 43.1 | 43 |
| Carbon number (%) | | | | | | |
| C24 | | 0.07 | 0.08 | 0.07 | | |
| C26 | 0.25 | 0.44 | 0.41 | 0.41 | 0.37 | 0.42 |
| C28 | 0.28 | 0.44 | 0.40 | 0.41 | 0.40 | 0.44 |
| C30 | 0.49 | 0.95 | 0.94 | 1.02 | 1.15 | 1.27 |
| C32 | 2.27 | 1.75 | 1.41 | 1.14 | 1.28 | 1.17 |
| C34 | 2.90 | 2.29 | 1.82 | 1.60 | 1.60 | 1.52 |
| C36 | 7.33 | 6.10 | 5.07 | 4.57 | 4.44 | 4.23 |
| C38 | 5.74 | 5.28 | 4.63 | 4.38 | 4.23 | 4.21 |
| C40 | 3.51 | 4.83 | 5.10 | 5.54 | 6.14 | 6.38 |
| C42 | 3.36 | 5.11 | 5.64 | 6.27 | 7.05 | 7.39 |
| C44 | 2.60 | 5.29 | 6.36 | 7.42 | 8.69 | 9.28 |
| C46 | 3.44 | 6.75 | 8.21 | 9.54 | 11.01 | 11.73 |
| C48 | 19.81 | 18.11 | 18.00 | 17.57 | 16.99 | 16.60 |
| C50 | 25.59 | 22.89 | 22.37 | 21.43 | 19.97 | 19.19 |
| C52 | 16.80 | 14.98 | 14.87 | 14.11 | 12.90 | 12.34 |
| C54 | 5.13 | 4.43 | 4.37 | 4.09 | 3.56 | 3.37 |
| >C54 | 0.49 | 0.28 | 0.31 | 0.36 | 0.22 | 0.46 |
| CN44 + 46 | 6.04 | 12.04 | 14.57 | 16.96 | 19.70 | 21.02 |

The reaction did not reach completion after 10 hours using a dosage of 0.24 wt % of once reused Test Enzyme (effective dosage 0.12 wt 1) at 80° C.

### Trial 15: 0.24 wt % Dosage of Test Enzyme I at 80° C.; 65:35 Blend, Enzyme Twice Reused

Trial 15 was performed using the same procedure as described for Trial 13, except the enzyme was 0.24 wt % dosage of the twice previously used Test Enzyme I from Trial 14 (previously used in Trials 13 and 14).

The profile of the oil produced by Trial 15 is set forth below in Table 17.

TABLE 17
Test Enzyme I, 0.24 wt % @ 80° C.
(65:35 blend), enzyme twice reused

| | Time (hours) | | | | | |
|---|---|---|---|---|---|---|
| | 0 | 2 | 4 | 6 | 8 | 10 |
| SFC | | | | | | |
| 10.0° C. | 69.7 | 68.7 | 69.1 | 69.3 | 69.5 | 69.6 |
| 21.1° C. | 41.5 | 40.0 | 40.9 | 41.2 | 41.4 | 41.7 |
| 26.7° C. | 30.7 | 28.5 | 28.1 | 27.9 | 27.9 | 28.1 |
| 33.3° C. | 20.5 | 17.7 | 16.5 | 16.1 | 15.7 | 14.8 |
| 40.0° C. | 13.5 | 8.9 | 7.9 | 6.1 | 5.0 | 4.4 |
| Dropping point (° C.) | 51.3 | 44.4 | 43.8 | 43 | 42.5 | 42 |
| Carbon number (%) | | | | | | |
| C24 | | | | 0.05 | 0.07 | |
| C26 | 0.25 | 0.42 | 0.36 | 0.39 | 0.39 | 0.49 |
| C28 | 0.28 | 0.42 | 0.37 | 0.41 | 0.39 | 0.50 |
| C30 | 0.49 | 0.99 | 0.99 | 1.05 | 1.08 | 1.34 |
| C32 | 2.27 | 1.64 | 1.90 | 0.99 | 0.72 | 0.81 |
| C34 | 2.90 | 2.25 | 1.79 | 1.34 | 1.16 | 1.15 |
| C36 | 7.33 | 5.94 | 4.94 | 4.34 | 3.76 | 3.58 |
| C38 | 5.74 | 5.11 | 4.53 | 4.06 | 3.80 | 3.87 |
| C40 | 3.51 | 5.10 | 5.53 | 5.90 | 6.19 | 6.75 |
| C42 | 3.36 | 5.48 | 6.22 | 6.85 | 7.35 | 7.99 |
| C44 | 2.60 | 5.83 | 7.22 | 8.42 | 9.42 | 10.36 |
| C46 | 3.44 | 7.44 | 9.34 | 10.87 | 12.15 | 13.17 |
| C48 | 19.81 | 17.69 | 16.95 | 17.04 | 16.75 | 16.13 |
| C50 | 25.59 | 22.37 | 21.29 | 20.52 | 19.80 | 18.36 |
| C52 | 16.80 | 14.72 | 14.19 | 13.55 | 13.00 | 11.95 |
| C54 | 5.13 | 4.33 | 4.13 | 3.90 | 3.65 | 3.22 |
| >C54 | 0.49 | 0.26 | 0.26 | 0.25 | 0.23 | 0.33 |
| CN44 + 46 | 6.04 | 13.27 | 16.56 | 19.29 | 21.57 | 23.52 |

The reaction did not reach completion after 10 hours using a dosage of 0.24 wt % of twice reused Test Enzyme I (effective dosage 0.08 wt.) at 80° C.

### Trial 16: 0.24 wt % Dosage of Test Enzyme I at 80° C.; 65:35 Blend

Trial 16 was performed using the same procedure as described for Trial 13.

The profile of the oil produced by Trial 16 is set forth below in Table 18.

TABLE 18
Test Enzyme I, 0.24 wt % @ 80° C. (65:35 blend)

| | Time (hours) | | | | | |
|---|---|---|---|---|---|---|
| | 0 | 2 | 4 | 6 | 8 | 10 |
| SFC | | | | | | |
| 10.0° C. | 69.7 | 70.8 | 71.9 | 73.0 | 73.0 | 73.2 |
| 21.1° C. | 41.5 | 44.3 | 44.6 | 45.1 | 45.1 | 45.0 |
| 26.7° C. | 30.7 | 31.4 | 31.0 | 31.2 | 30.8 | 31.0 |
| 33.3° C. | 20.5 | 17.7 | 16.7 | 16.0 | 15.2 | 14.9 |
| 40.0° C. | 13.5 | 6.5 | 4.5 | 3.9 | 3.3 | 3.3 |
| Dropping point (° C.) | 51.3 | 43.2 | 42.3 | 41.9 | 41.1 | 41.2 |
| Carbon number (%) | | | | | | |
| C24 | | 0.05 | 0.04 | 0.05 | | |
| C26 | 0.25 | 0.22 | 0.23 | 0.23 | 0.22 | 0.22 |
| C28 | 0.28 | 0.27 | 0.24 | 0.25 | 0.25 | 0.23 |
| C30 | 0.49 | 0.83 | 0.84 | 1.00 | 0.87 | 0.86 |
| C32 | 2.27 | 1.50 | 1.13 | 1.11 | 1.05 | 1.00 |
| C34 | 2.90 | 1.22 | 0.77 | 0.62 | 0.54 | 0.47 |
| C36 | 7.33 | 3.42 | 2.78 | 2.24 | 2.11 | 2.08 |
| C38 | 5.74 | 3.38 | 3.31 | 2.82 | 2.57 | 2.73 |
| C40 | 3.51 | 5.83 | 5.99 | 6.16 | 6.16 | 6.06 |
| C42 | 3.36 | 6.66 | 7.43 | 7.74 | 7.91 | 7.90 |
| C44 | 2.60 | 8.90 | 10.75 | 11.80 | 12.39 | 12.65 |
| C46 | 3.44 | 11.68 | 14.12 | 15.46 | 16.30 | 16.67 |
| C48 | 19.81 | 17.26 | 16.69 | 16.49 | 16.34 | 16.32 |
| C50 | 25.59 | 20.96 | 19.81 | 19.09 | 18.75 | 18.53 |
| C52 | 16.80 | 13.42 | 12.47 | 11.86 | 11.61 | 11.42 |
| C54 | 5.13 | 3.58 | 3.14 | 2.87 | 2.74 | 2.68 |
| >C54 | 0.49 | 0.79 | 0.21 | 0.17 | 0.18 | 0.18 |
| CN44 + 46 | 6.04 | 20.58 | 24.87 | 27.27 | 28.69 | 29.32 |

The reaction reached completion after 6 hours using a dosage of 0.24 wt % of Test Enzyme I at 80° C.

### Trial 17: 0.24 wt % Dosage of Test Enzyme I at 85° C.; 65:35 Blend

Trial 17 was performed using the same procedure as described for Trial 13, except the temperature was 85° C.

The profile of the oil produced by Trial 17 is set forth below in Table 19.

TABLE 19
Test Enzyme I, 0.24 wt % @ 85° C. (65:35 blend)

| | Time (hours) | | | | | |
|---|---|---|---|---|---|---|
| | 0 | 2 | 4 | 6 | 8 | 10 |
| SFC | | | | | | |
| 10.0° C. | 69.7 | 70.6 | 71.7 | 72.2 | 72.3 | 72.6 |
| 21.1° C. | 41.5 | 42.9 | 42.9 | 43.0 | 43.2 | 43.1 |
| 26.7° C. | 30.7 | 29.0 | 28.7 | 28.7 | 28.9 | 28.4 |
| 33.3° C. | 20.5 | 16.1 | 14.7 | 14.3 | 13.6 | 13.5 |
| 40.0° C. | 13.5 | 5.0 | 3.7 | 2.9 | 2.6 | 2.3 |
| Dropping point (° C.) | 51.3 | 42.6 | 41.9 | 41.4 | 40.9 | 40.8 |
| Carbon number (%) | | | | | | |
| C24 | | | | | | |
| C26 | 0.25 | 0.23 | 0.22 | 0.25 | 0.23 | 0.23 |
| C28 | 0.28 | 0.24 | 0.25 | 0.27 | 0.26 | 0.28 |
| C30 | 0.49 | 0.83 | 0.84 | 0.91 | 0.90 | 0.97 |
| C32 | 2.27 | 1.36 | 1.19 | 1.19 | 1.13 | 1.17 |
| C34 | 2.90 | 1.24 | 0.87 | 1.11 | 0.73 | 0.58 |
| C36 | 7.33 | 3.74 | 3.18 | 2.70 | 2.49 | 2.30 |
| C38 | 5.74 | 3.96 | 3.51 | 3.34 | 2.88 | 3.10 |
| C40 | 3.51 | 6.19 | 6.51 | 6.63 | 6.64 | 6.61 |
| C42 | 3.36 | 7.24 | 7.91 | 8.20 | 8.38 | 8.45 |
| C44 | 2.60 | 9.39 | 10.97 | 11.78 | 12.40 | 12.73 |
| C46 | 3.44 | 12.17 | 14.24 | 15.29 | 16.10 | 16.58 |
| C48 | 19.81 | 16.90 | 16.36 | 15.97 | 15.97 | 15.77 |
| C50 | 25.59 | 19.99 | 18.79 | 18.04 | 17.77 | 17.44 |
| C52 | 16.80 | 12.83 | 11.89 | 11.30 | 11.04 | 10.80 |
| C54 | 5.13 | 3.46 | 3.04 | 2.76 | 2.67 | 2.57 |
| >C54 | 0.49 | 0.23 | 0.20 | 0.25 | 0.41 | 0.39 |
| CN44 + 46 | 6.04 | 21.56 | 25.22 | 27.07 | 28.50 | 29.31 |

The reaction reached completion after 6 hours using a dosage of 0.24 wt % of Test Enzyme I at 85° C.

### Trial 18: 0.24 wt % Dosage of Test Enzyme I at 90° C.; 65:35 Blend

Trial 18 was performed using the same procedure as described for Trial 13, except the temperature was 90° C.

The profile of the oil produced by Trial 18 is set forth below in Table 20.

TABLE 20
Test Enzyme I, 0.24 wt % @ 90° C. (65:35 blend)

| | Time (hours) | | | | | |
|---|---|---|---|---|---|---|
| | 0 | 2 | 4 | 6 | 8 | 10 |
| SFC | | | | | | |
| 10.0° C. | 69.7 | 71.2 | 70.6 | 71.1 | 70.7 | 70.97 |
| 21.1° C. | 41.5 | 42.7 | 42.8 | 42.8 | 42.8 | 43.0 |
| 26.7° C. | 30.7 | 29.1 | 28.9 | 28.8 | 28.7 | 28.5 |
| 33.3° C. | 20.5 | 15.6 | 14.7 | 14.4 | 14.1 | 13.9 |
| 40.0° C. | 13.5 | 5.0 | 3.9 | 3.2 | 3.2 | 2. |
| Dropping point (° C.) | 51.3 | 43 | 42.1 | 41.3 | 41.2 | 40.8 |
| Carbon number (%) | | | | | | |
| C24 | | | | | | |
| C26 | 0.25 | 0.23 | 0.23 | 0.22 | 0.23 | 0.22 |
| C28 | 0.28 | 0.25 | 0.28 | 0.27 | 0.27 | 0.26 |
| C30 | 0.49 | 0.87 | 0.92 | 0.93 | 0.94 | 0.96 |
| C32 | 2.27 | 1.35 | 1.24 | 1.19 | 1.16 | 1.16 |
| C34 | 2.90 | 1.08 | 0.82 | 0.70 | 0.63 | 0.93 |
| C36 | 7.33 | 3.48 | 2.82 | 2.58 | 2.40 | 2.32 |
| C38 | 5.74 | 3.79 | 3.16 | 3.00 | 3.14 | 3.06 |
| C40 | 3.51 | 6.21 | 6.46 | 6.53 | 6.50 | 6.49 |
| C42 | 3.36 | 7.33 | 7.85 | 8.10 | 8.18 | 8.25 |
| C44 | 2.60 | 9.74 | 11.07 | 11.82 | 12.17 | 12.50 |
| C46 | 3.44 | 12.70 | 14.43 | 15.42 | 15.89 | 16.31 |
| C48 | 19.81 | 16.74 | 16.35 | 16.15 | 16.00 | 15.82 |
| C50 | 25.59 | 19.88 | 18.98 | 18.44 | 18.07 | 17.71 |
| C52 | 16.80 | 12.69 | 11.97 | 11.54 | 11.33 | 10.99 |
| C54 | 5.13 | 3.33 | 3.02 | 2.82 | 2.71 | 2.61 |
| >C54 | 0.49 | 0.33 | 0.41 | 0.28 | 0.38 | 0.41 |
| CN44 + 46 | 6.04 | 22.44 | 25.50 | 27.24 | 28.06 | 28.81 |

The reaction reached completion after 6 hours using a dosage of 0.24 wt % of Test Enzyme I at 90° C.

### Trial 19: 0.24 wt % Dosage of Test Enzyme I at 95° C.; 65:35 Blend

Trial 19 was performed using the same procedure as described for Trial 13, except the temperature was 95° C.

The profile of the oil produced by Trial 19 is set forth below in Table 21.

TABLE 21
Test Enzyme I, 0.24 wt % @ 95° C. (65:35 blend)

| | Time (hours) | | | | | |
|---|---|---|---|---|---|---|
| | 0 | 2 | 4 | 6 | 8 | 10 |
| SFC | | | | | | |
| 10.0° C. | 69.7 | 70.0 | 69.9 | 70.3 | 71.3 | 71.3 |
| 21.1° C. | 41.5 | 42.1 | 42.1 | 42.2 | 42.6 | 42.9 |
| 26.7° C. | 30.7 | 28.9 | 28.7 | 28.2 | 28.7 | 28.6 |
| 33.3° C. | 20.5 | 16.0 | 14.9 | 14.2 | 14.4 | 14.1 |
| 40.0° C. | 13.5 | 5.9 | 4.4 | 3.7 | 3.3 | 3.1 |
| Dropping point (° C.) | 51.3 | 43.5 | 42 | 41.4 | 41.5 | 41.2 |
| Carbon number (%) | | | | | | |
| C24 | | | | | | |
| C26 | 0.25 | 0.24 | 0.25 | 0.22 | 0.24 | 0.24 |
| C28 | 0.28 | 0.27 | 0.28 | 0.27 | 0.28 | 0.27 |
| C30 | 0.49 | 0.88 | 0.88 | 0.93 | 0.94 | 0.94 |
| C32 | 2.27 | 1.54 | 1.33 | 1.28 | 1.23 | 1.18 |
| C34 | 2.90 | 1.35 | 1.06 | 0.84 | 0.77 | 0.65 |
| C36 | 7.33 | 3.86 | 3.33 | 2.93 | 2.61 | 2.71 |
| C38 | 5.74 | 3.92 | 3.73 | 3.48 | 3.12 | 3.23 |
| C40 | 3.51 | 6.08 | 6.46 | 6.50 | 6.62 | 6.56 |
| C42 | 3.36 | 6.93 | 7.65 | 7.88 | 8.14 | 8.14 |
| C44 | 2.60 | 8.65 | 10.26 | 10.94 | 11.57 | 11.90 |
| C46 | 3.44 | 11.25 | 13.31 | 14.27 | 15.06 | 15.52 |
| C48 | 19.81 | 17.12 | 16.45 | 16.17 | 16.16 | 16.00 |
| C50 | 25.59 | 20.60 | 19.18 | 18.76 | 18.37 | 18.08 |
| C52 | 16.80 | 13.30 | 12.22 | 11.89 | 11.58 | 11.35 |
| C54 | 5.13 | 3.62 | 3.15 | 3.02 | 2.87 | 2.78 |
| >C54 | 0.49 | 0.39 | 0.46 | 0.62 | 0.44 | 0.44 |
| CN44 + 46 | 6.04 | 19.90 | 23.58 | 25.21 | 26.63 | 27.42 |

The reaction reached completion after 10 hours using a dosage of 0.24 wt of Test Enzyme I at 95° C. The decrease in reaction rate in comparison to Trials 16-18 is the result enzyme denaturation.

### Trial 20: 0.36 wt % Dosage of Test Enzyme I at 80° C.; 65:35 Blend

Trial 20 was performed using the same procedure as described for Trial 13, except 0.36 wt % of the Test Enzyme I was used.

The profile of the oil produced by Trial 20 is set forth below in Table 22.

TABLE 22
Test Enzyme I, 0.36 wt % @ 80° C. (65:35 blend)

| | Time (hours) | | | | | |
|---|---|---|---|---|---|---|
| | 0 | 2 | 4 | 6 | 8 | 10 |
| SFC | | | | | | |
| 10.0° C. | 69.7 | 71.4 | 72.4 | 72.7 | 72.7 | 72.9 |
| 21.1° C. | 41.5 | 43.1 | 43.4 | 43.7 | 44.3 | 44.1 |
| 26.7° C. | 30.7 | 29.3 | 29.3 | 29.2 | 29.6 | 29.7 |
| 33.3° C. | 20.5 | 15.2 | 14.0 | 13.8 | 13.9 | 13.6 |
| 40.0° C. | 13.5 | 4.4 | 3.3 | 2.6 | 2.1 | 1.9 |
| Dropping point (° C.) | 51.3 | 42.2 | 41.1 | 41 | 40.7 | 40.4 |
| Carbon number (%) | | | | | | |
| C24 | | | | | | |
| C26 | 0.25 | 0.21 | 0.24 | 0.22 | 0.26 | 0.21 |
| C28 | 0.28 | 0.24 | 0.24 | 0.24 | 0.28 | 0.24 |
| C30 | 0.49 | 0.89 | 0.92 | 0.91 | 0.96 | 0.95 |
| C32 | 2.27 | 1.23 | 1.10 | 1.06 | 1.10 | 1.08 |
| C34 | 2.90 | 1.34 | 0.93 | 1.17 | 0.61 | 0.97 |
| C36 | 7.33 | 2.98 | 2.26 | 2.11 | 1.96 | 1.95 |
| C38 | 5.74 | 3.39 | 2.99 | 2.58 | 2.43 | 2.77 |
| C40 | 3.51 | 6.25 | 6.34 | 6.32 | 6.34 | 6.25 |
| C42 | 3.36 | 7.63 | 8.09 | 8.21 | 8.32 | 8.25 |
| C44 | 2.60 | 10.84 | 12.33 | 12.90 | 13.26 | 13.24 |
| C46 | 3.44 | 14.15 | 16.12 | 16.91 | 17.32 | 17.36 |
| C48 | 19.81 | 16.37 | 15.97 | 15.92 | 15.99 | 15.91 |
| C50 | 25.59 | 19.05 | 18.02 | 17.65 | 17.54 | 17.28 |
| C52 | 16.80 | 12.06 | 11.30 | 10.91 | 10.80 | 10.68 |
| C54 | 5.13 | 3.05 | 2.68 | 2.56 | 2.51 | 2.48 |
| >C54 | 0.49 | 0.34 | 0.47 | 0.34 | 0.34 | 0.37 |
| CN44 + 46 | 6.04 | 24.98 | 28.44 | 29.80 | 30.58 | 30.60 |

The reaction reached completion after 4 hours using a low dosage of 0.36 wt % of Test Enzyme I at 80° C.

### Trial 21: 0.48 wt % Dosage of Test Enzyme I at 80° C.; 65:35 Blend

Trial 21 was performed using the same procedure as described for Trial 13, except 0.48 wt % of the Test Enzyme I was used. Once the immobilized enzyme was removed from the oil, it was kept in a sealed container for reuse.

The profile of the oil produced by Trial 21 is set forth below in Table 23.

TABLE 23
Test Enzyme I, 0.48 wt % @ 80° C. (65:35 blend)

| | Time (hours) | | | | | |
|---|---|---|---|---|---|---|
| | 0 | 2 | 4 | 6 | 8 | 10 |
| SFC | | | | | | |
| 10.0° C. | 69.7 | 69.1 | 69.0 | 69.5 | 69.3 | 69.4 |
| 21.1° C. | 41.5 | 39.5 | 40.3 | 40.7 | 41.1 | 41.2 |
| 26.7° C. | 30.7 | 26.1 | 26.2 | 26.5 | 26.8 | 27.3 |
| 33.3° C. | 20.5 | 12.5 | 11.6 | 11.7 | 11.8 | 11.9 |
| 40.0° C. | 13.5 | 1.9 | 0.7 | 0.7 | 0.7 | 0.8 |
| Dropping point (° C.) | 51.3 | 40.3 | 39.6 | 39.5 | 39.5 | 39.7 |
| Carbon number (%) | | | | | | |
| C24 | 0.25 | 0.38 | 0.36 | 0.27 | 0.34 | 0.35 |
| C26 | 0.28 | 0.40 | 0.36 | 0.33 | 0.42 | 0.32 |
| C28 | 0.49 | 1.35 | 1.38 | 1.31 | 1.43 | 1.33 |
| C30 | 2.27 | 1.57 | 1.48 | 1.44 | 1.70 | 1.47 |
| C32 | 2.90 | 1.59 | 1.74 | 2.06 | 1.80 | 1.58 |
| C34 | 7.33 | 1.47 | 1.64 | 1.53 | 1.65 | 1.66 |
| C36 | 5.74 | 2.91 | 3.14 | 2.98 | 3.02 | 3.01 |
| C38 | 3.51 | 6.48 | 6.44 | 6.33 | 6.30 | 6.30 |
| C40 | 3.36 | 8.20 | 8.43 | 8.39 | 8.36 | 8.37 |
| C42 | 2.60 | 12.10 | 12.90 | 13.01 | 12.99 | 13.05 |
| C44 | 3.44 | 15.97 | 17.02 | 17.31 | 17.17 | 17.34 |
| C46 | 19.81 | 15.59 | 15.23 | 15.38 | 15.36 | 15.50 |
| C48 | 25.59 | 17.52 | 16.47 | 16.31 | 16.18 | 16.31 |
| C50 | 16.80 | 11.20 | 10.47 | 10.42 | 10.40 | 10.55 |
| C52 | 5.13 | 2.81 | 2.52 | 2.51 | 2.53 | 2.57 |
| C54 | 0.49 | 0.46 | 0.43 | 0.42 | 0.35 | 0.30 |
| >C54 | 0.25 | 0.38 | 0.36 | 0.27 | 0.34 | 0.35 |
| CN44 + 46 | 6.04 | 28.07 | 29.92 | 30.32 | 30.16 | 30.38 |

The reaction reached completion after 2 hours using a low dosage of 0.48 wt % of Test Enzyme at 80° C.

### Trial 22: 0.48 wt % Dosage of Test Enzyme at 80° C.; 65:35 Blend, Enzyme Reused

Trial 22 was performed using the same procedure as described for Trial 21, except the enzyme was a 0.48 wt % of the previously used Test Enzyme from Trial 21 plus an additional 33 wt % of fresh (unused) Test Enzyme (based on the weight of the previously used Test Enzyme). Once the immobilized enzyme was removed from the oil, it was kept in a sealed container for reuse.

The profile of the oil produced by Trial 22 is set forth below in Table 24.

TABLE 24
Test Enzyme I, 0.48 wt % @ 80° C. (65:35 blend), enzyme reused

| | Time (hours) | | | | | |
|---|---|---|---|---|---|---|
| | 0 | 2 | 4 | 6 | 8 | 10 |
| SFC | | | | | | |
| 10.0° C. | 69.7 | 69.7 | 70.6 | 71.1 | 71.1 | 70.9 |
| 21.1° C. | 41.5 | 41.5 | 41.6 | 41.9 | 42.0 | 42.1 |
| 26.7° C. | 30.7 | 26.8 | 27.2 | 27.0 | 27.5 | 27.8 |
| 33.3° C. | 20.5 | 12.6 | 11.9 | 11.7 | 11.7 | 11.9 |
| 40.0° C. | 13.5 | 1.9 | 0.9 | 0.8 | 1.0 | 0.9 |
| Dropping point (° C.) | 51.3 | 40.7 | 40.1 | 39.8 | 40 | 39.2 |
| Carbon number (%) | | | | | | |
| C24 | | | | | | |
| C26 | 0.25 | 0.23 | 0.28 | 0.25 | 0.25 | 0.26 |
| C28 | 0.28 | 0.29 | 0.31 | 0.25 | 0.34 | 0.30 |
| C30 | 0.49 | 1.07 | 1.02 | 0.93 | 1.08 | 1.06 |
| C32 | 2.27 | 1.33 | 1.20 | 1.29 | 1.31 | 1.23 |
| C34 | 2.90 | 1.17 | 1.27 | 1.29 | 1.25 | 1.20 |
| C36 | 7.33 | 1.15 | 1.09 | 1.16 | 1.22 | 0.98 |
| C38 | 5.74 | 3.62 | 3.26 | 3.15 | 3.12 | 3.03 |
| C40 | 3.51 | 6.92 | 6.84 | 6.78 | 6.72 | 6.73 |
| C42 | 3.36 | 8.62 | 8.87 | 8.91 | 8.87 | 8.90 |
| C44 | 2.60 | 12.00 | 13.09 | 13.42 | 13.47 | 13.56 |
| C46 | 3.44 | 15.63 | 17.16 | 17.61 | 17.78 | 17.86 |
| C48 | 19.81 | 15.76 | 15.45 | 15.48 | 15.42 | 15.58 |
| C50 | 25.59 | 17.63 | 16.66 | 16.39 | 16.17 | 16.17 |
| C52 | 16.80 | 11.34 | 10.60 | 10.42 | 10.31 | 10.33 |
| C54 | 5.13 | 2.88 | 2.56 | 2.49 | 2.47 | 2.50 |
| >C54 | 0.49 | 0.37 | 0.34 | 0.17 | 0.22 | 0.30 |
| CN44 + 46 | 6.04 | 27.62 | 30.25 | 31.04 | 31.25 | 31.43 |

The reaction reached completion after 2 hours using a dosage of 0.48 wt % of once reused Test Enzyme I in combination with an additional 33% of fresh (unused) Test Enzyme (effective dosage 0.32 wt %) at 80° C.

### Trial 23: 0.48 wt % Dosage of Test Enzyme I at 80° C.; 65:35 Blend, Enzyme Reused 2 Times

Trial 23 was performed using the same procedure as described for Trial 21, except the enzyme was a 0.48 wt % of the twice previously used Test Enzyme I from Trial 22 (used in Trials 21 and 22) plus an additional 25 wt % of fresh (unused) Test Enzyme I (based on the weight of the previously used Test Enzyme I). Once the immobilized enzyme was removed from the oil, it was kept in a sealed container for reuse.

The profile of the oil produced by Trial 23 is set forth below in Table 25.

TABLE 25
Test Enzyme I, 0.48 wt % @ 80° C.
(65:35 blend), enzyme twice reused

| | Time (hours) | | | | | |
|---|---|---|---|---|---|---|
| | 0 | 2 | 4 | 6 | 8 | 10 |
| SFC | | | | | | |
| 10.0° C. | 69.7 | 70.7 | 71.9 | 72.1 | 72.7 | 71.8 |
| 21.1° C. | 41.5 | 42.9 | 43.5 | 43.4 | 43.8 | 44.2 |
| 26.7° C. | 30.7 | 28.9 | 28.7 | 28.7 | 29.3 | 30.0 |
| 33.3° C. | 20.5 | 14.3 | 13.5 | 13.2 | 13.5 | 13.5 |
| 40.0° C. | 13.5 | 3.1 | 2.1 | 1.6 | 1.8 | 1.8 |
| Dropping point (° C.) | 51.3 | 41.2 | 40.3 | 40.4 | 40.4 | 40.2 |
| Carbon number (%) | | | | | | |
| C24 | | | | | | |
| C26 | 0.25 | 0.23 | 0.25 | 0.26 | 0.35 | 0.27 |
| C28 | 0.28 | 0.27 | 0.28 | 0.25 | 0.35 | 0.25 |
| C30 | 0.49 | 0.97 | 1.02 | 1.05 | 1.08 | 1.04 |
| C32 | 2.27 | 1.33 | 1.15 | 1.13 | 1.27 | 1.17 |
| C34 | 2.90 | 1.40 | 1.26 | 1.37 | 1.46 | 1.28 |
| C36 | 7.33 | 1.06 | 1.04 | 1.15 | 1.30 | 1.13 |
| C38 | 5.74 | 3.31 | 2.93 | 2.96 | 2.99 | 2.85 |
| C40 | 3.51 | 6.59 | 6.52 | 6.48 | 6.52 | 6.49 |
| C42 | 3.36 | 8.23 | 8.42 | 8.46 | 8.49 | 8.53 |
| C44 | 2.60 | 12.08 | 13.14 | 13.41 | 13.45 | 13.55 |
| C46 | 3.44 | 15.66 | 17.09 | 17.55 | 17.54 | 17.63 |
| C48 | 19.81 | 16.14 | 15.98 | 15.77 | 15.58 | 15.90 |
| C50 | 25.59 | 18.25 | 17.40 | 16.93 | 16.64 | 16.77 |
| C52 | 16.80 | 11.47 | 10.74 | 10.47 | 10.32 | 10.44 |
| C54 | 5.13 | 2.82 | 2.50 | 2.42 | 2.40 | 2.45 |
| >C54 | 0.49 | 0.19 | 0.29 | 0.33 | 0.26 | 0.26 |
| CN44 + 46 | 6.04 | 27.74 | 30.23 | 30.96 | 30.99 | 31.18 |

The reaction reached completion after 2 hours using a dosage of 0.48 wt % of twice reused Test Enzyme I in combination with an additional 25% of fresh (unused) Test Enzyme I (based on the weight of the previously used Test Enzyme I) (effective dosage 0.27 wt %) at 80° C.

### Trial 24: 0.48 wt % Dosage of Test Enzyme I at 80° C.; 65:35 Blend, Enzyme Reused 3 Times

Trial 24 was performed using the same procedure as described for Trial 21, except the enzyme was a 0.48 wt % of the three times previously used Test Enzyme I from Trial 23 (used in Trials 21, 22, and 23) plus an additional 20 wt % of fresh (unused) Test Enzyme I (based on the weight of the previously used Test Enzyme I). Once the immobilized enzyme was removed from the oil, it was kept in a sealed container for reuse.

The profile of the oil produced by Trial 24 is set forth below in Table 26.

TABLE 26
Test Enzyme I, 0.48 wt % @ 80° C.
(65:35 blend), enzyme reused 3 times

| | Time (hours) | | | | | |
|---|---|---|---|---|---|---|
| | 0 | 2 | 4 | 6 | 8 | 10 |
| SFC | | | | | | |
| 10.0° C. | 69.7 | 70.7 | 71.2 | 72.0 | 71.3 | 71.4 |
| 21.1° C. | 41.5 | 41.6 | 41.8 | 42.7 | 42.9 | 43.0 |
| 26.7° C. | 30.7 | 27.5 | 27.3 | 27.9 | 28.3 | 28.3 |
| 33.3° C. | 20.5 | 13.4 | 12.7 | 12.0 | 12.6 | 12.8 |
| 40.0° C. | 13.5 | 2.2 | 1.2 | 1.7 | 1.3 | 1.2 |
| Dropping point (° C.) | 51.3 | 41 | 39.9 | 40.2 | 40.1 | 40.2 |
| Carbon number (%) | | | | | | |
| C24 | | | | | | |
| C26 | 0.25 | 0.27 | 0.24 | 0.28 | 0.22 | 0.20 |
| C28 | 0.28 | 0.27 | 0.27 | 0.28 | 0.24 | 0.29 |
| C30 | 0.49 | 1.00 | 1.00 | 1.02 | 0.99 | 1.00 |
| C32 | 2.27 | 1.23 | 1.15 | 1.23 | 1.17 | 1.42 |
| C34 | 2.90 | 1.22 | 1.21 | 1.34 | 1.39 | 1.36 |
| C36 | 7.33 | 0.98 | 1.00 | 1.15 | 1.15 | 1.09 |
| C38 | 5.74 | 3.29 | 2.99 | 3.04 | 3.01 | 3.05 |
| C40 | 3.51 | 6.70 | 6.62 | 6.57 | 6.55 | 6.58 |
| C42 | 3.36 | 8.43 | 8.64 | 8.66 | 8.67 | 8.61 |
| C44 | 2.60 | 12.14 | 13.16 | 13.37 | 13.47 | 13.47 |
| C46 | 3.44 | 15.87 | 17.28 | 17.55 | 17.78 | 17.72 |
| C48 | 19.81 | 16.01 | 15.73 | 15.68 | 15.70 | 15.73 |
| C50 | 25.59 | 17.94 | 17.11 | 16.68 | 16.56 | 16.44 |
| C52 | 16.80 | 11.49 | 10.81 | 10.52 | 10.40 | 10.36 |
| C54 | 5.13 | 2.85 | 2.58 | 2.49 | 2.47 | 2.47 |
| >C54 | 0.49 | 0.30 | 0.21 | 0.15 | 0.23 | 0.24 |
| CN44 + 46 | 6.04 | 28.02 | 30.45 | 30.92 | 31.25 | 31.19 |

The reaction reached completion after 2 hours using a dosage of 0.48 wt % of three times reused Test Enzyme I in combination with an additional 20% of fresh (unused) Test Enzyme I (based on the weight of the previously used Test Enzyme I) (effective dosage 0.24 wt %) at 80° C.

### Trial 25: 0.48 wt % Dosage of Test Enzyme I at 80° C.; 65:35 Blend, Enzyme Reused 4 Times

Trial 25 was performed using the same procedure as described for Trial 21, except the enzyme was a 0.48 wt % of the four times previously used Test Enzyme I from Trial 24 (used in Trials 21, 22, 23, and 24) plus an additional 17 wt % of fresh (unused) Test Enzyme I (based on the weight of the previously used Test Enzyme I).

The profile of the oil produced by Trial 25 is set forth below in Table 27.

TABLE 27
Test Enzyme I, 0.48 wt % @ 80° C.
(65:35 blend), enzyme reused 4 times

| | Time (hours) | | | | | |
|---|---|---|---|---|---|---|
| | 0 | 2 | 4 | 6 | 8 | 10 |
| SFC | | | | | | |
| 10.0° C. | 69.7 | 72.1 | 72.6 | 72.0 | 72.0 | 72.3 |
| 21.1° C. | 41.5 | 43.4 | 43.7 | 44.0 | 44.1 | 44.4 |
| 26.7° C. | 30.7 | 29.3 | 29.2 | 29.9 | 29.6 | 29.3 |
| 33.3° C. | 20.5 | 13.7 | 13.3 | 13.7 | 13.5 | 13.4 |
| 40.0° C. | 13.5 | 2.5 | 1.8 | 1.4 | 1.9 | 1.9 |
| Dropping point (° C.) | 51.3 | 40.8 | 40.7 | 41.6 | 40.5 | 40.5 |
| Carbon number (%) | | | | | | |
| C24 | | | | | | |
| C26 | 0.25 | 0.26 | 0.27 | 0.26 | 0.25 | 0.22 |
| C28 | 0.28 | 0.28 | 0.28 | 0.27 | 0.28 | 0.27 |
| C30 | 0.49 | 1.03 | 1.05 | 1.08 | 1.10 | 1.14 |
| C32 | 2.27 | 1.17 | 1.16 | 1.19 | 1.15 | 1.14 |
| C34 | 2.90 | 1.24 | 1.32 | 1.34 | 1.31 | 1.44 |
| C36 | 7.33 | 1.03 | 1.07 | 1.07 | 1.09 | 1.28 |
| C38 | 5.74 | 2.99 | 2.86 | 2.83 | 2.94 | 2.96 |
| C40 | 3.51 | 6.54 | 6.50 | 6.45 | 6.45 | 6.43 |
| C42 | 3.36 | 8.39 | 8.44 | 8.48 | 8.49 | 8.45 |
| C44 | 2.60 | 12.87 | 13.42 | 13.51 | 13.56 | 13.50 |
| C46 | 3.44 | 16.66 | 17.41 | 17.65 | 17.62 | 17.61 |
| C48 | 19.81 | 16.06 | 15.85 | 15.84 | 15.93 | 15.86 |
| C50 | 25.59 | 17.71 | 17.19 | 16.89 | 16.90 | 16.68 |
| C52 | 16.80 | 10.92 | 10.53 | 10.42 | 10.48 | 10.38 |
| C54 | 5.13 | 2.60 | 2.45 | 2.42 | 2.44 | 2.44 |
| >C54 | 0.49 | 0.25 | 0.19 | 0.30 | 0.00 | 0.19 |
| CN44 + 46 | 6.04 | 29.53 | 30.83 | 31.17 | 31.18 | 31.11 |

The reaction reached completion after 2 hours using a dosage of 0.48 wt % of four times previously used Test Enzyme I in combination with an additional 17% of fresh (unused) Test Enzyme I (effective dosage 0.23 wt %) at 80° C.

A second immobilization carrier was employed for the next series of examples Test Enzyme II.

### Trial 26: 0.48 wt % Dosage of Test Enzyme II at 80° C.; 65:35 Blend

Trial 26 was performed using the same procedure as described for Trial 21, except the dosage was 0.48 wt % and utilized Test Enzyme II.

The profile of the oil produced by Trial 26 is set forth below in Table 28.

TABLE 28
Test Enzyme II, 0.48 wt % @ 80° C. (65:35 blend)

| | Time (hours) | | | | | |
|---|---|---|---|---|---|---|
| | 0 | 2 | 4 | 6 | 8 | 10 |
| SFC | | | | | | |
| 10.0° C. | 73.0 | 74.4 | 75.5 | 75.2 | 75.2 | 76.3 |
| 21.1° C. | 44.8 | 44.4 | 45.4 | 46.0 | 46.5 | 46.8 |
| 26.7° C. | 33.8 | 30.9 | 32.0 | 32.4 | 33.2 | 32.9 |
| 33.3° C. | 21.8 | 13.7 | 14.5 | 14.6 | 13.7 | 14.5 |
| 40.0° C. | 13.7 | 2.9 | 2.6 | 2.3 | 2.4 | 2.2 |
| Dropping point (° C.) | 51.7 | 41.0 | 40.4 | 40.5 | 40.7 | 40.5 |
| Carbon number (%) | | | | | | |
| C24 | 0.03 | 0.02 | 0.02 | 0.03 | 0.04 | 0.03 |
| C26 | 0.08 | 0.03 | 0.03 | 0.01 | 0.04 | 0.01 |
| C28 | 0.11 | 0.04 | 0.04 | 0.03 | 0.05 | 0.07 |
| C30 | 0.38 | 0.10 | 0.08 | 0.07 | 0.07 | 0.07 |
| C32 | 2.00 | 0.41 | 0.31 | 0.30 | 0.30 | 0.29 |
| C34 | 2.69 | 0.69 | 0.54 | 0.51 | 0.51 | 0.50 |
| C36 | 6.75 | 2.34 | 1.92 | 1.89 | 1.91 | 1.87 |
| C38 | 5.04 | 2.86 | 2.57 | 2.52 | 2.55 | 2.52 |
| C40 | 3.05 | 6.86 | 6.75 | 6.68 | 6.82 | 6.79 |
| C42 | 2.84 | 8.50 | 8.62 | 8.64 | 8.70 | 8.70 |
| C44 | 2.15 | 12.94 | 13.92 | 14.04 | 14.18 | 14.25 |
| C46 | 3.08 | 16.56 | 17.88 | 18.11 | 18.19 | 18.05 |
| C48 | 20.20 | 16.31 | 16.34 | 16.31 | 16.38 | 16.53 |
| C50 | 28.50 | 18.39 | 17.73 | 17.49 | 17.23 | 17.22 |
| C52 | 17.54 | 11.16 | 10.70 | 10.73 | 10.48 | 10.50 |
| C54 | 5.12 | 2.57 | 2.38 | 2.40 | 2.36 | 2.42 |
| >C54 | 0.42 | 0.21 | 0.17 | 0.25 | 0.19 | 0.20 |
| CN44 + 46 | 5.23 | 29.50 | 31.80 | 32.15 | 32.37 | 32.29 |

The reaction did reached completion after only two hours using a dosage of 0.48 wt % of Test Enzyme II at 80° C.

### Trial 27: 0.48 wt % Dosage of Test Enzyme II at 80° C.; 65:35 Blend, Enzyme Reused

Trial 27 was performed using the same procedure as described for Trial 26, except the enzyme was a 0.48 wt % of the previously used Test Enzyme from Trial 26 plus an additional 33 wt % of fresh (unused) Test Enzyme II (based on the weight of the previously used Test Enzyme). Once the immobilized enzyme was removed from the oil, it was kept in a sealed container for reuse.

The profile of the oil produced by Trial 27 is set forth below in Table 29.

TABLE 29
Test Enzyme II, 0.48 wt % @ 80°
C. (65:35 blend), enzyme reused

| | Time (hours) | | | | | |
|---|---|---|---|---|---|---|
| | 0 | 2 | 4 | 6 | 8 | 10 |
| SFC | | | | | | |
| 10.0° C. | 73.0 | 73.8 | 74.3 | 74.9 | 74.3 | 75.1 |
| 21.1° C. | 44.8 | 44.1 | 44.8 | 45.4 | 45.2 | 45.9 |
| 26.7° C. | 33.8 | 31.1 | 31.1 | 31.8 | 31.9 | 32.4 |
| 33.3° C. | 21.8 | 14.9 | 14.0 | 13.6 | 14.0 | 14.2 |
| 40.0° C. | 13.7 | 2.8 | 1.9 | 2.0 | 1.8 | 2.3 |
| Dropping point (° C.) | 51.70 | 40.9 | 40.3 | 40.4 | 40.0 | 40.3 |
| Carbon number (%) | | | | | | |
| C24 | 0.03 | 0.03 | 0.04 | 0.02 | 0.03 | 0.02 |
| C26 | 0.08 | 0.03 | 0.03 | 0.02 | 0.03 | 0.03 |
| C28 | 0.11 | 0.08 | 0.07 | 0.04 | 0.06 | 0.05 |
| C30 | 0.38 | 0.11 | 0.08 | 0.08 | 0.07 | 0.08 |
| C32 | 2.00 | 0.43 | 0.32 | 0.31 | 0.31 | 0.30 |
| C34 | 2.69 | 0.72 | 0.56 | 0.54 | 0.53 | 0.52 |
| C36 | 6.75 | 2.41 | 2.01 | 1.96 | 1.96 | 1.94 |
| C38 | 5.04 | 2.91 | 2.64 | 2.61 | 2.62 | 2.60 |
| C40 | 3.05 | 6.91 | 6.87 | 6.89 | 6.94 | 6.91 |
| C42 | 2.84 | 8.56 | 8.85 | 8.81 | 8.91 | 8.91 |
| C44 | 2.15 | 12.81 | 13.86 | 14.14 | 14.25 | 14.24 |
| C46 | 3.08 | 16.34 | 17.83 | 18.12 | 18.17 | 18.12 |
| C48 | 20.20 | 16.41 | 16.13 | 16.25 | 16.26 | 16.29 |
| C50 | 28.50 | 18.20 | 17.45 | 17.20 | 16.94 | 16.88 |
| C52 | 17.54 | 11.15 | 10.62 | 10.45 | 10.36 | 10.38 |
| C54 | 5.12 | 2.66 | 2.41 | 2.35 | 2.35 | 2.40 |
| >C54 | 0.42 | 0.24 | 0.23 | 0.17 | 0.19 | 0.34 |
| CN44 + 46 | 5.23 | 29.15 | 31.69 | 32.27 | 32.43 | 32.36 |

The reaction reached completion after 2 hours using a dosage of 0.48 wt % of once reused Test Enzyme II in combination with an additional 33% of fresh (unused) Test Enzyme II (effective dosage 0.32 wt %) at 80° C.

Trial 28: 0.48 wt % Dosage of Test Enzyme II at 80° C.; 65:35 Blend, Enzyme Reused 2 times

Trial 28 was performed using the same procedure as described for Trial 27, except the enzyme was a 0.48 wt % of the twice previously used Test Enzyme II from Trial 27 (used in Trials 26 and 27) plus an additional 25 wt % of fresh (unused) Test Enzyme II (based on the weight of the previously used Test Enzyme II). Once the immobilized enzyme was removed from the oil, it was kept in a sealed container for reuse.

The profile of the oil produced by Trial 28 is set forth below in Table 30.

TABLE 30
Test Enzyme II, 0.48 wt % @ 80° C.
(65:35 blend), enzyme twice reused

| | Time (hours) | | | | | |
|---|---|---|---|---|---|---|
| | 0 | 2 | 4 | 6 | 8 | 10 |
| SFC | | | | | | |
| 10.0° C. | 73.0 | 73.6 | 74.1 | 74.7 | 74.2 | 74.2 |
| 21.1° C. | 44.8 | 42.2 | 44.4 | 45.0 | 44.9 | 45.5 |
| 26.7° C. | 33.8 | 31.3 | 30.5 | 31.3 | 31.7 | 32.3 |
| 33.3° C. | 21.8 | 14.5 | 13.6 | 13.8 | 14.3 | 13.9 |
| 40.0° C. | 13.7 | 2.9 | 2.4 | 2.5 | 2.2 | 2.4 |
| Dropping point (° C.) | 51.70 | 41 | 40.4 | 40.3 | 40.3 | 40.5 |
| Carbon number (%) | | | | | | |
| C24 | 0.03 | 0.03 | 0.03 | 0.02 | 0.03 | 0.01 |
| C26 | 0.08 | 0.03 | 0.04 | 0.03 | 0.04 | 0.03 |
| C28 | 0.11 | 0.08 | 0.06 | 0.09 | 0.09 | 0.10 |
| C30 | 0.38 | 0.10 | 0.07 | 0.07 | 0.07 | 0.06 |
| C32 | 2.00 | 0.40 | 0.30 | 0.28 | 0.28 | 0.27 |
| C34 | 2.69 | 0.68 | 0.54 | 0.50 | 0.49 | 0.48 |
| C36 | 6.75 | 2.26 | 1.90 | 1.82 | 1.81 | 1.80 |
| C38 | 5.04 | 2.76 | 2.51 | 2.45 | 2.45 | 2.43 |
| C40 | 3.05 | 6.68 | 6.60 | 6.57 | 6.60 | 6.58 |
| C42 | 2.84 | 8.27 | 8.51 | 8.53 | 8.56 | 8.55 |
| C44 | 2.15 | 12.56 | 13.59 | 13.90 | 13.94 | 13.97 |
| C46 | 3.08 | 16.18 | 17.66 | 18.01 | 18.03 | 18.05 |
| C48 | 20.20 | 16.46 | 16.22 | 16.40 | 16.37 | 16.52 |
| C50 | 28.50 | 18.99 | 18.08 | 17.81 | 17.65 | 17.57 |
| C52 | 17.54 | 11.62 | 11.07 | 10.89 | 10.85 | 10.86 |
| C54 | 5.12 | 2.72 | 2.49 | 2.47 | 2.47 | 2.51 |
| >C54 | 0.42 | 0.19 | 0.35 | 0.16 | 0.24 | 0.15 |
| CN44 + 46 | 5.23 | 28.74 | 31.25 | 31.92 | 31.97 | 32.02 |

The reaction reached completion after 2 hours using a dosage of 0.48 wt % of twice reused Test Enzyme II in combination with an additional 25% of fresh (unused) Test Enzyme II (based on the weight of the previously used Test Enzyme II) (effective dosage 0.27 wt %) at 80° C.

### Trial 29: 0.48 wt % Dosage of Test Enzyme II at 80° C.; 65:35 Blend, Enzyme Reused 3 Times

Trial 29 was performed using the same procedure as described for Trial 26, except the enzyme was a 0.48 wt % of the three times previously used Test Enzyme II from Trial 30 (used in Trials 26, 27, and 28) plus an additional 20 wt % of fresh (unused) Test Enzyme II (based on the weight of the previously used Test Enzyme II). Once the immobilized enzyme was removed from the oil, it was kept in a sealed container for reuse.

The profile of the oil produced by Trial 29 is set forth below in Table 31.

TABLE 31
Test Enzyme II, 0.48 wt % @ 80° C.
(65:35 blend), enzyme reused 3 times

| | Time (hours) | | | | | |
|---|---|---|---|---|---|---|
| | 0 | 2 | 4 | 6 | 8 | 10 |
| SFC | | | | | | |
| 10.0° C. | 73.0 | 73.0 | 74.1 | 73.6 | 73.8 | 73.8 |
| 21.1° C. | 44.8 | 42.5 | 43.8 | 43.5 | 44.0 | 43.9 |
| 26.7° C. | 33.8 | 28.9 | 29.7 | 30.0 | 30.1 | 30.0 |
| 33.3° C. | 21.8 | 13.2 | 12.7 | 12.4 | 12.9 | 13.0 |
| 40.0° C. | 13.7 | 2.5 | 1.4 | 1.4 | 1.4 | 1.4 |
| Dropping point (° C.) | 51.70 | 40.4 | 39.8 | 39.5 | 39.9 | 39.7 |
| Carbon number (%) | | | | | | |
| C24 | 0.03 | 0.03 | 0.03 | 0.06 | 0.03 | 0.03 |
| C26 | 0.08 | 0.05 | 0.08 | 0.13 | 0.05 | 0.06 |
| C28 | 0.11 | 0.09 | 0.06 | 0.06 | 0.09 | 0.04 |
| C30 | 0.38 | 0.11 | 0.08 | 0.08 | 0.08 | 0.07 |
| C32 | 2.00 | 0.45 | 0.34 | 0.29 | 0.32 | 0.32 |
| C34 | 2.69 | 0.74 | 0.59 | 0.55 | 0.56 | 0.55 |
| C36 | 6.75 | 2.47 | 2.08 | 2.03 | 2.02 | 2.00 |
| C38 | 5.04 | 2.99 | 2.71 | 2.65 | 2.69 | 2.67 |
| C40 | 3.05 | 6.97 | 6.99 | 6.97 | 7.01 | 7.00 |
| C42 | 2.84 | 8.63 | 9.03 | 9.01 | 9.02 | 9.08 |
| C44 | 2.15 | 12.56 | 13.81 | 13.93 | 14.06 | 14.08 |
| C46 | 3.08 | 16.27 | 17.87 | 18.09 | 18.22 | 18.20 |
| C48 | 20.20 | 16.04 | 15.93 | 15.90 | 16.00 | 16.12 |
| C50 | 28.50 | 18.24 | 17.12 | 16.96 | 16.72 | 16.62 |
| C52 | 17.54 | 11.37 | 10.58 | 10.55 | 10.50 | 10.46 |
| C54 | 5.12 | 2.73 | 2.44 | 2.44 | 2.45 | 2.46 |
| >C54 | 0.42 | 0.23 | 0.24 | 0.30 | 0.20 | 0.24 |
| CN44 + 46 | 5.23 | 28.83 | 31.67 | 32.01 | 32.28 | 32.27 |

The reaction reached completion after 2 hours using a dosage of 0.48 wt % of three times reused Test Enzyme II in combination with an additional 20% of fresh (unused) Test Enzyme II (based on the weight of the previously used Test Enzyme II) (effective dosage 0.24 wt %) at 80° C.

### Trial 30: 0.48 wt % Dosage of Test Enzyme II at 80° C.; 65:35 Blend, Enzyme Reused 4 Times

Trial 30 was performed using the same procedure as described for Trial 26, except the enzyme was a 0.48 wt % of the four times previously used Test Enzyme II from Trial 31 (used in Trials 26, 27, 28, and 29) plus an additional 17 wt % of fresh (unused) Test Enzyme II (based on the weight of the previously used Test Enzyme II).

The profile of the oil produced by Trial 30 is set forth below in Table 32.

TABLE 32
Test Enzyme II, 0.48 wt % @ 80° C.
(65:35 blend), enzyme reused 4 times

| | Time (hours) | | | | | |
|---|---|---|---|---|---|---|
| | 0 | 2 | 4 | 6 | 8 | 10 |
| SFC | | | | | | Sample |
| | | | | | | Lost |
| 10.0° C. | 73.0 | 74.3 | 74.8 | 75.3 | 75.0 | |
| 21.1° C. | 44.8 | 44.5 | 45.0 | 45.0 | 45.5 | |
| 26.7° C. | 33.8 | 31.1 | 31.5 | 31.8 | 32.2 | |
| 33.3° C. | 21.8 | 14.0 | 13.9 | 13.9 | 14.0 | |
| 40.0° C. | 13.7 | 3.3 | 2.5 | 2.4 | 2.1 | |
| Dropping point (° C.) | 51.7 | 40.8 | 40.2 | 40.3 | 40.3 | |
| Carbon number (%) | | | | | | |
| C24 | 0.03 | 0.03 | 0.05 | 0.05 | 0.04 | |
| C26 | 0.08 | 0.09 | 0.10 | 0.10 | 0.08 | |
| C28 | 0.11 | 0.07 | 0.06 | 0.05 | 0.04 | |
| C30 | 0.38 | 0.10 | 0.08 | 0.07 | 0.07 | |
| C32 | 2.00 | 0.41 | 0.32 | 0.30 | 0.30 | |
| C34 | 2.69 | 0.69 | 0.56 | 0.53 | 0.52 | |
| C36 | 6.75 | 2.30 | 1.97 | 1.92 | 1.90 | |
| C38 | 5.04 | 2.82 | 2.62 | 2.57 | 2.56 | |
| C40 | 3.05 | 6.84 | 6.88 | 6.80 | 6.82 | |
| C42 | 2.84 | 8.50 | 8.70 | 8.76 | 8.74 | |
| C44 | 2.15 | 12.84 | 13.84 | 13.94 | 14.20 | |
| C46 | 3.08 | 16.38 | 17.68 | 18.02 | 18.07 | |
| C48 | 20.20 | 16.41 | 16.20 | 16.05 | 16.34 | |
| C50 | 28.50 | 18.49 | 17.62 | 17.49 | 17.23 | |
| C52 | 17.54 | 11.18 | 10.57 | 10.68 | 10.46 | |
| C54 | 5.12 | 2.59 | 2.39 | 2.41 | 2.38 | |
| >C54 | 0.42 | 0.25 | 0.37 | 0.26 | 0.26 | |
| CN44 + 46 | 5.23 | 29.23 | 31.53 | 31.95 | 32.27 | |

The reaction reached completion after 2 hours using a dosage of 0.48 wt % of four times previously used Test Enzyme II in combination with an additional 17% of fresh (unused) Test Enzyme II (effective dosage 0.23 wt % o) at 80° C.

### Trial 31: 0.48 wt % Dosage of Test Enzyme 11 at 80° C.; 65:35 Blend

Trial 31 was performed using the same procedure as described for Trial 26, except sampling began at 0.5 hours. Samples were taken at 0.5, 1.0, 1.5, 2.0, and 4.0 hours.

The profile of the oil produced by Trial 31 is set forth below in Table 33.

TABLE 33
Test Enzyme II, 0.48 wt % @ 80° C. (65:35 blend)

| | Time (hours) | | | | | |
|---|---|---|---|---|---|---|
| | 0 | 0.5 | 1.0 | 1.5 | 2.0 | 4.0 |
| SFC | | | | | | |
| 10.0° C. | 73.0 | 72.4 | 73.5 | 74.5 | 74.9 | 75.2 |
| 21.1° C. | 44.8 | 42.6 | 43.7 | 44.0 | 44.1 | 44.2 |
| 26.7° C. | 33.8 | 28.9 | 28.5 | 28.4 | 28.5 | 28.6 |
| 33.3° C. | 21.8 | 17.2 | 15.4 | 14.5 | 13.7 | 12.8 |
| 40.0° C. | 13.7 | 7.5 | 4.6 | 3.8 | 3.1 | 1.7 |
| Dropping point (° C.) | 51.7 | 43.3 | 42.0 | 41.3 | 40.9 | 40.0 |
| Carbon number (%) | | | | | | |
| <C24 | 0.03 | 0.04 | 0.03 | 0.04 | 0.04 | 0.03 |
| C24 | 0.03 | | 0.03 | | 0.05 | |
| C26 | 0.08 | 0.07 | 0.11 | 0.09 | 0.09 | 0.06 |
| C28 | 0.11 | 0.04 | 0.03 | 0.04 | 0.03 | 0.03 |
| C30 | 0.38 | 0.41 | 0.08 | 0.04 | 0.06 | 0.03 |
| C32 | 2.00 | 1.01 | 0.53 | 0.36 | 0.23 | 0.20 |
| C34 | 2.69 | 1.47 | 0.92 | 0.67 | 0.51 | 0.30 |
| C36 | 6.75 | 4.34 | 3.12 | 2.62 | 2.28 | 1.80 |
| C38 | 5.04 | 3.87 | 3.33 | 3.02 | 2.81 | 2.50 |
| C40 | 3.05 | 5.62 | 6.23 | 6.73 | 6.83 | 6.83 |
| C42 | 2.84 | 6.28 | 7.62 | 8.06 | 8.08 | 8.97 |
| C44 | 2.15 | 8.00 | 10.75 | 11.78 | 12.58 | 13.66 |
| C46 | 3.08 | 10.31 | 13.89 | 15.38 | 16.52 | 18.13 |
| C48 | 20.20 | 17.94 | 17.35 | 17.03 | 16.86 | 16.58 |
| C50 | 28.50 | 22.61 | 20.39 | 19.72 | 19.14 | 17.91 |
| C52 | 17.54 | 14.10 | 12.48 | 11.66 | 11.46 | 10.72 |
| C54 | 5.12 | 3.71 | 2.94 | 2.64 | 2.34 | 2.16 |
| >C54 | 0.42 | 0.17 | 0.16 | 0.12 | 0.09 | 0.08 |
| CN44 + 46 | 5.23 | 18.31 | 24.64 | 27.16 | 29.10 | 31.79 |

The reaction did reached completion after only one and a half (1.5) hours using a dosage of 0.48 wt % of Test Enzyme II at 80° C.

### Trial 32: 0.48 wt % Dosage of Test Enzyme II at 80° C.; 65:35 Blend, Enzyme Reused

Trial 32 was performed using the same procedure as described for Trial 31, except the enzyme was a 0.48 wt % of the previously used Test Enzyme from Trial 32 plus an additional 33 wt % of fresh (unused) Test Enzyme II (based on the weight of the previously used Test Enzyme). Once the immobilized enzyme was removed from the oil, it was kept in a sealed container for reuse.

The profile of the oil produced by Trial 32 is set forth below in Table 34.

TABLE 34
Test Enzyme II, 0.48 wt % @ 80°
C. (65:35 blend), enzyme reused

| | Time (hours) | | | | | |
|---|---|---|---|---|---|---|
| | 0 | 0.5 | 1.0 | 1.5 | 2.0 | 4.0 |
| SFC | | | | Missed | | |
| | | | | Sample | | |
| | | | | Time | | |
| 10.0° C. | 73.0 | 72.0 | 73.6 | | 74.7 | 75.2 |
| 21.1° C. | 44.8 | 42.5 | 43.5 | | 43.9 | 44.2 |
| 26.7° C. | 33.8 | 28.8 | 28.9 | | 28.9 | 28.2 |
| 33.3° C. | 21.8 | 16.5 | 15.1 | | 13.8 | 13.4 |
| 40.0° C. | 13.7 | 6.2 | 4.1 | | 2.4 | 1.8 |
| Dropping point (° C.) | 51.7 | 42.8 | 41.6 | | 40.5 | 40.2 |
| Carbon number (%) | | | | | | |
| <C24 | 0.03 | 0.03 | 0.03 | | 0.03 | 0.06 |
| C24 | 0.03 | | | | | 0.04 |
| C26 | 0.08 | 0.07 | 0.08 | | 0.06 | 0.08 |
| C28 | 0.11 | 0.03 | 0.03 | | 0.04 | 0.03 |
| C30 | 0.38 | 0.10 | 0.06 | | 0.04 | 0.04 |
| C32 | 2.00 | 0.80 | 0.47 | | 0.26 | 0.10 |
| C34 | 2.69 | 1.02 | 0.75 | | 0.43 | 0.30 |
| C36 | 6.75 | 3.73 | 2.56 | | 2.08 | 1.71 |
| C38 | 5.04 | 3.71 | 2.94 | | 2.30 | 2.41 |
| C40 | 3.05 | 6.15 | 6.59 | | 6.77 | 6.45 |
| C42 | 2.84 | 6.90 | 8.07 | | 8.62 | 8.55 |
| C44 | 2.15 | 9.09 | 11.41 | | 13.38 | 14.32 |
| C46 | 3.08 | 11.44 | 14.62 | | 17.30 | 18.50 |
| C48 | 20.20 | 17.84 | 17.11 | | 16.31 | 16.59 |
| C50 | 28.50 | 22.07 | 20.10 | | 18.67 | 17.85 |
| C52 | 17.54 | 13.56 | 12.22 | | 11.28 | 10.80 |
| C54 | 5.12 | 3.28 | 2.85 | | 2.30 | 2.09 |
| >C54 | 0.42 | 0.16 | 0.13 | | 0.12 | 0.08 |
| CN44 + 46 | 5.23 | 20.53 | 26.03 | | 30.68 | 32.82 |

The reaction reached completion after one and a half (1.5) hours using a dosage of 0.48 wt % of once reused Test Enzyme II in combination with an additional 33% of fresh (unused) Test Enzyme II (effective dosage 0.32 wt %) at 80° C.

### Trial 33: 0.48 wt % Dosage of Test Enzyme II at 80° C.; 65:35 Blend, Enzyme Reused 2 Times

Trial 33 was performed using the same procedure as described for Trial 31, except the enzyme was a 0.48 wt % of the previously used Test Enzyme from Trial 32 (used enzyme in Trials 31 and 32) plus an additional 25 wt % of fresh (unused) Test Enzyme II (based on the weight of the previously used Test Enzyme). Once the immobilized enzyme was removed from the oil, it was kept in a sealed container for reuse.

The profile of the oil produced by Trial 33 is set forth below in Table 35.

TABLE 35
Test Enzyme II, 0.48 wt % @ 80° C.
(65:35 blend), enzyme twice reused

| | Time (hours) | | | | | |
|---|---|---|---|---|---|---|
| | 0 | 0.5 | 1.0 | 1.5 | 2.0 | 4.0 |
| SFC | | | | | | |
| 10.0° C. | 73.0 | 73.1 | 72.0 | 73.1 | 73.9 | 74.7 |
| 21.1° C. | 44.8 | 42.7 | 42.5 | 43.3 | 43.5 | 43.9 |
| 26.7° C. | 33.8 | 28.6 | 28.2 | 28.4 | 28.4 | 28.4 |
| 33.3° C. | 21.8 | 16.4 | 14.9 | 14.5 | 13.7 | 13.3 |
| 40.0° C. | 13.7 | 6.6 | 3.8 | 3.3 | 2.9 | 1.6 |
| Dropping point (° C.) | 51.7 | 42.9 | 41.7 | 41.1 | 40.9 | 39.9 |
| Carbon number (%) | | | | | | |
| <C24 | 0.03 | 0.04 | 0.04 | 0.05 | 0.06 | 0.05 |
| C24 | 0.03 | 0.03 | 0.03 | 0.03 | 0.04 | 0.05 |
| C26 | 0.08 | 0.08 | 0.12 | 0.08 | 0.10 | 0.12 |
| C28 | 0.11 | 0.03 | 0.03 | 0.03 | 0.05 | 0.02 |
| C30 | 0.38 | 0.09 | 0.06 | 0.03 | 0.13 | 0.13 |
| C32 | 2.00 | 0.51 | 0.33 | 0.19 | 0.08 | 0.23 |
| C34 | 2.69 | 0.95 | 0.66 | 0.47 | 0.22 | 0.37 |
| C36 | 6.75 | 3.95 | 2.95 | 2.38 | 2.02 | 1.65 |
| C38 | 5.04 | 3.75 | 3.20 | 2.82 | 2.62 | 2.25 |
| C40 | 3.05 | 6.14 | 6.74 | 6.81 | 6.94 | 6.69 |
| C42 | 2.84 | 6.49 | 7.86 | 8.50 | 8.74 | 8.83 |
| C44 | 2.15 | 8.55 | 10.79 | 12.32 | 13.22 | 14.16 |
| C46 | 3.08 | 11.51 | 14.24 | 15.52 | 17.26 | 18.34 |
| C48 | 20.20 | 18.29 | 17.22 | 16.98 | 15.99 | 16.42 |
| C50 | 28.50 | 22.69 | 20.41 | 19.42 | 18.84 | 17.73 |
| C52 | 17.54 | 13.70 | 12.40 | 11.75 | 11.34 | 10.60 |
| C54 | 5.12 | 3.13 | 2.81 | 2.45 | 2.23 | 2.26 |
| >C54 | 0.42 | 0.10 | 0.11 | 0.16 | 0.12 | 0.10 |
| CN44 + 46 | 5.23 | 20.06 | 25.03 | 27.84 | 30.48 | 32.50 |

The reaction reached completion after one and a half (1.5) hours using a dosage of 0.48 wt % of twice reused Test Enzyme II in combination with an additional 25% of fresh (unused) Test Enzyme II (effective dosage 0.27 wt %) at 80° C.

### Trial 34: 0.48 wt % Dosage of Test Enzyme II at 80° C.; 65:35 Blend, Enzyme Reused 3 Times

Trial 34 was performed using the same procedure as described for Trial 31, except the enzyme was a 0.48 wt % of the previously used Test Enzyme from Trial 33 (used enzyme in Trials 31, 32, and 33) plus an additional 20 wt % of fresh (unused) Test Enzyme II (based on the weight of the previously used Test Enzyme). Once the immobilized enzyme was removed from the oil, it was kept in a sealed container for reuse.

The profile of the oil produced by Trial 34 is set forth below in Table 36.

TABLE 36
Test Enzyme II, 0.48 wt % @ 80° C.
(65:35 blend), enzyme reused 3 times

| | Time (hours) | | | | | |
|---|---|---|---|---|---|---|
| | 0 | 0.5 | 1.0 | 1.5 | 2.0 | 4.0 |
| SFC | | | | | | |
| 10.0° C. | 73.0 | 72.2 | 73.1 | 74.0 | 73.0 | 73.2 |
| 21.1° C. | 44.8 | 42.0 | 42.6 | 42.9 | 42.9 | 42.8 |
| 26.7° C. | 33.8 | 28.0 | 27.9 | 27.9 | 28.1 | 28.0 |
| 33.3° C. | 21.8 | 15.8 | 14.6 | 14.1 | 13.6 | 12.9 |
| 40.0° C. | 13.7 | 5.6 | 4.2 | 2.8 | 2.4 | 1.2 |
| Dropping point (° C.) | 51.7 | 42.6 | 41.5 | 40.8 | 40.7 | 39.7 |
| Carbon number (%) | | | | | | |
| <C24 | 0.03 | 0.04 | 0.06 | 0.05 | 0.03 | 0.06 |
| C24 | 0.03 | 0.04 | 0.04 | 0.03 | 0.03 | 0.04 |
| C26 | 0.08 | 0.09 | 0.17 | 0.15 | 0.07 | 0.17 |
| C28 | 0.11 | 0.04 | 0.03 | 0.03 | 0.04 | 0.04 |
| C30 | 0.38 | 0.08 | 0.08 | 0.09 | 0.03 | 0.04 |
| C32 | 2.00 | 0.81 | 0.56 | 0.37 | 0.17 | 0.26 |
| C34 | 2.69 | 1.22 | 0.88 | 0.71 | 0.37 | 0.45 |
| C36 | 6.75 | 3.82 | 3.01 | 2.55 | 2.19 | 1.92 |
| C38 | 5.04 | 3.80 | 3.33 | 3.03 | 2.74 | 2.58 |
| C40 | 3.05 | 6.33 | 6.79 | 6.96 | 6.96 | 6.97 |
| C42 | 2.84 | 7.18 | 7.59 | 8.41 | 8.47 | 8.92 |
| C44 | 2.15 | 9.05 | 10.81 | 12.18 | 12.81 | 14.09 |
| C46 | 3.08 | 11.75 | 14.14 | 15.71 | 16.82 | 18.01 |
| C48 | 20.20 | 17.61 | 16.96 | 16.50 | 16.54 | 16.18 |
| C50 | 28.50 | 21.30 | 20.19 | 18.92 | 18.77 | 17.55 |
| C52 | 17.54 | 13.34 | 12.38 | 11.60 | 11.32 | 10.60 |
| C54 | 5.12 | 3.32 | 2.84 | 2.59 | 2.57 | 2.08 |
| >C54 | 0.42 | 0.17 | 0.15 | 0.12 | 0.08 | 0.05 |
| CN44 + 46 | 5.23 | 20.80 | 24.95 | 27.89 | 29.63 | 32.10 |

The reaction reached completion after one and a half (1.5) hours using a dosage of 0.48 wt % of three times reused Test Enzyme II in combination with an additional 20% of fresh (unused) Test Enzyme II (effective dosage 0.24 wt %) at 80° C.

### Trial 35: 0.48 wt % Dosage of Test Enzyme II at 80° C.; 65:35 Blend, Enzyme Reused 4 Times

Trial 35 was performed using the same procedure as described for Trial 31, except the enzyme was a 0.48 wt % of the previously used Test Enzyme from Trial 34 (used enzyme in Trials 31, 32, 33, and 34) plus an additional 17 wt % of fresh (unused) Test Enzyme II (based on the weight of the previously used Test Enzyme). Once the immobilized enzyme was removed from the oil, it was kept in a sealed container for reuse.

The profile of the oil produced by Trial 35 is set forth below in Table 37.

TABLE 37
Test Enzyme II, 0.48 wt % @ 80° C.
(65:35 blend), enzyme reused 4 times

| | Time (hours) | | | | | |
|---|---|---|---|---|---|---|
| | 0 | 0.5 | 1.0 | 1.5 | 2.0 | 4.0 |
| SFC | | | | | | |
| 10.0° C. | 73.0 | 72.2 | 73.2 | 74.1 | 74.7 | 73.8 |
| 21.1° C. | 44.8 | 42.8 | 43.1 | 43.7 | 44.1 | 44.1 |
| 26.7° C. | 33.8 | 28.8 | 28.3 | 28.4 | 28.5 | 28.5 |
| 33.3° C. | 21.8 | 16.0 | 14.7 | 13.8 | 14.1 | 13.4 |
| 40.0° C. | 13.7 | 5.7 | 3.8 | 2.7 | 2.4 | 1.5 |
| Dropping point (° C.) | 51.7 | 42.8 | 41.1 | 41.4 | 40.6 | 40.6 |
| Carbon number (%) | | | | | | |
| <C24 | 0.03 | 0.04 | 0.04 | 0.05 | 0.04 | 0.05 |
| C24 | 0.03 | 0.03 | 0.03 | 0.05 | 0.03 | 0.03 |
| C26 | 0.08 | 0.14 | 0.15 | 0.16 | 0.19 | 0.17 |
| C28 | 0.11 | 0.03 | 0.03 | 0.03 | 0.04 | 0.03 |
| C30 | 0.38 | 0.13 | 0.12 | 0.11 | 0.06 | 0.09 |
| C32 | 2.00 | 0.75 | 0.51 | 0.39 | 0.34 | 0.25 |
| C34 | 2.69 | 1.09 | 0.81 | 0.63 | 0.57 | 0.45 |
| C36 | 6.75 | 3.53 | 2.78 | 2.38 | 2.12 | 1.97 |
| C38 | 5.04 | 3.6 | 3.04 | 2.75 | 2.68 | 2.5 |
| C40 | 3.05 | 6.34 | 6.8 | 6.81 | 6.88 | 6.89 |
| C42 | 2.84 | 7.3 | 8.06 | 8.49 | 8.6 | 8.7 |
| C44 | 2.15 | 9.5 | 11.49 | 12.64 | 13.25 | 14.08 |
| C46 | 3.08 | 12.26 | 14.73 | 16.23 | 16.96 | 18.19 |
| C48 | 20.20 | 17.56 | 16.83 | 16.57 | 16.4 | 16.26 |
| C50 | 28.50 | 21.35 | 19.74 | 18.84 | 18.49 | 17.55 |
| C52 | 17.54 | 13.03 | 11.91 | 11.33 | 10.89 | 10.52 |
| C54 | 5.12 | 3.21 | 2.77 | 2.46 | 2.35 | 2.17 |
| >C54 | 0.42 | 0.14 | 0.15 | 0.09 | 0.09 | 0.09 |
| CN44 + 46 | 5.23 | 21.76 | 26.22 | 28.87 | 30.21 | 32.27 |

The reaction reached completion after one and a half (1.5) hours using a dosage of 0.48 wt % of four times reused Test Enzyme II in combination with an additional 17% o of fresh (unused) Test Enzyme II (effective dosage 0.23 wt % o) at 80° C.

### Trial 36: 0.48 wt % Dosage of Test Enzyme II at 80° C.; 65:35 Blend, Enzyme Reused 5 Times

Trial 36 was performed using the same procedure as described for Trial 31, except the enzyme was a 0.48 wt % of the previously used Test Enzyme from Trial 35 (used enzyme in Trials 31, 32, 33, 34, and 35). No additional fresh enzyme was used.

The profile of the oil produced by Trial 36 is set forth below in Table 38.

TABLE 38
Test Enzyme II, 0.48 wt % @ 80° C.
(65:35 blend), enzyme reused 5 times

| | Time (hours) | | | | | |
|---|---|---|---|---|---|---|
| | 0 | 0.5 | 1.0 | 1.5 | 2.0 | 4.0 |
| SFC | | | | | | |
| 10.0° C. | 73.0 | 72.1 | 73.1 | 73.7 | 73.8 | 74.4 |
| 21.1° C. | 44.8 | 42.7 | 43.3 | 43.4 | 43.6 | 43.2 |
| 26.7° C. | 33.8 | 29.1 | 28.9 | 28.5 | 28.3 | 27.8 |
| 33.3° C. | 21.8 | 17.1 | 15.6 | 15.2 | 14.6 | 13.3 |
| 40.0° C. | 13.7 | 6.9 | 4.7 | 3.8 | 3.4 | 2.3 |
| Dropping point (° C.) | 51.7 | 43.4 | 42.8 | 41.7 | 41.7 | 40.2 |
| Carbon number (%) | | | | | | |
| <C24 | 0.03 | 0.04 | 0.04 | 0.05 | 0.05 | 0.06 |
| C24 | 0.03 | 0.03 | 0.05 | 0.06 | 0.02 | 0.09 |
| C26 | 0.08 | 0.21 | 0.16 | 0.2 | 0.19 | 0.23 |
| C28 | 0.11 | 0.09 | 0.08 | 0.03 | 0.03 | 0.03 |
| C30 | 0.38 | 0.15 | 0.66 | 0.14 | 0.14 | 0.13 |
| C32 | 2.00 | 1.00 | 1.01 | 0.56 | 0.47 | 0.37 |
| C34 | 2.69 | 1.52 | 3.26 | 0.86 | 0.7 | 0.58 |
| C36 | 6.75 | 4.23 | 3.47 | 2.84 | 2.57 | 2.05 |
| C38 | 5.04 | 4.01 | 6.54 | 3.25 | 3.02 | 2.66 |
| C40 | 3.05 | 5.97 | 2.05 | 6.82 | 6.86 | 6.86 |
| C42 | 2.84 | 6.58 | 5.43 | 8.02 | 8.20 | 8.65 |
| C44 | 2.15 | 8.00 | 9.96 | 11.09 | 11.78 | 13.39 |
| C46 | 3.08 | 10.35 | 12.83 | 14.19 | 15.24 | 17.33 |
| C48 | 20.20 | 17.97 | 17.51 | 16.93 | 16.62 | 16.28 |
| C50 | 28.50 | 22.34 | 21.1 | 19.9 | 19.29 | 18.02 |
| C52 | 17.54 | 13.77 | 12.83 | 12.09 | 11.79 | 10.78 |
| C54 | 5.12 | 3.54 | 3.01 | 2.83 | 2.92 | 2.32 |
| >C54 | 0.42 | 0.23 | | 0.15 | 0.11 | 0.15 |
| CN44 + 46 | 5.23 | 18.35 | 22.79 | 25.28 | 27.02 | 30.72 |

The reaction reached completion after two hours using a dosage of 0.48 wt % of five times reused Test Enzyme II (effective dosage 0.18 wt % o) at 80° C.

### Results Summary

The results of Trials 1-36 are summarized in Table 39 below.

TABLE 39

| | | | | Enzyme | | | |
|---|---|---|---|---|---|---|---|
| | | | | reuse | | | Reaction |
| | | | | (Trial no. | Effective | | completion |
| | | Oil | Dosage | of previous | Dosage | Temp. | (time to |
| Trial | Enzyme | blend | (wt %) | use) | (wt %) | (° C.) | completion) |
| 1 | Lipozyme | 85:15 | 0.12 | No | 0.12 | 70 | no |
| | TL-IM | | | | | | |
| 2 | Lipozyme | 85:15 | 0.24 | No | 0.24 | 70 | no |
| | TL-IM | | | | | | |
| 3 | Lipozyme | 85:15 | 0.48 | No | 0.48 | 70 | no |
| | TL-IM | | | | | | |
| 4 | Lipozyme | 85:15 | 0.72 | No | 0.72 | 70 | yes (8 hrs) |
| | TL-IM | | | | | | |
| 5 | Lipozyme | 85:15 | 0.72 | Yes (4) | 0.36 | 70 | no |
| | TL-IM | | | | | | |
| 6 | Test | 85:15 | 0.72 | No | 0.72 | 70 | yes (2 hrs) |
| | Enzyme I | | | | | | |
| 7 | Test | 85:15 | 0.72 | Yes (6) | 0.36 | 70 | yes (2 hrs) |
| | Enzyme I | | | | | | |
| 8 | Test | 85:15 | 0.72 | Yes (6, 7) | 0.24 | 70 | yes (4 hrs) |
| | Enzyme I | | | | | | |
| 9 | Lipozyme | 85:15 | 0.48 | No | 0.48 | 80 | yes (10 hrs) |
| | TL-IM | | | | | | |
| 10 | Lipozyme | 85:15 | 0.48 | No | 0.48 | 85 | yes (8 hrs) |
| | TL-IM | | | | | | |
| 11 | Lipozyme | 85:15 | 0.48 | No | 0.48 | 90 | yes (8 hrs) |
| | TL-IM | | | | | | |
| 12 | Lipozyme | 65:35 | 0.72 | No | 0.72 | 80 | Yes (8 hrs) |
| | TL-IM | | | | | | |
| 13 | Test | 65:35 | 0.24 | No | 0.24 | 80 | Yes (10 hrs) |
| | Enzyme I | | | | | | |
| 14 | Test | 65:35 | 0.24 | yes (13) | 0.12 | 80 | No |
| | Enzyme I | | | | | | |
| 15 | Test | 65:35 | 0.24 | yes (13, 14) | 0.08 | 80 | No |
| | Enzyme I | | | | | | |
| 16 | Test | 65:35 | 0.24 | No | 0.24 | 80 | Yes (6 hrs) |
| | Enzyme I | | | | | | |
| 17 | Test | 65:35 | 0.24 | No | 0.24 | 85 | Yes (6 hrs) |
| | Enzyme I | | | | | | |
| 18 | Test | 65:35 | 0.24 | No | 0.24 | 90 | Yes (6 hrs) |
| | Enzyme I | | | | | | |
| 19 | Test | 65:35 | 0.24 | No | 0.24 | 95 | Yes (10 hrs) |
| | Enzyme I | | | | | | |
| 20 | Test | 65:35 | 0.36 | No | 0.36 | 80 | Yes (4 hrs) |
| | Enzyme I | | | | | | |
| 21 | Test | 65:35 | 0.48 | No | 0.48 | 80 | Yes (2 hrs) |
| | Enzyme I | | | | | | |
| 22 | Test | 65:35 | 0.48 | yes (21 + | 0.32 | 80 | Yes (2 hrs) |
| | Enzyme I | | | 33% fresh) | | | |
| 23 | Test | 65:35 | 0.48 | yes (21, 22, + | 0.27 | 80 | Yes (2 hrs) |
| | Enzyme I | | | 25% fresh) | | | |
| 24 | Test | 65:35 | 0.48 | yes (21, 22, | 0.24 | 80 | Yes (2 hrs) |
| | Enzyme I | | | 23, + 20% | | | |
| | | | | fresh) | | | |
| 25 | Test | 65:35 | 0.48 | yes (21, 22, | 0.23 | 80 | Yes (2 hrs) |
| | Enzyme I | | | 23, 24, + | | | |
| | | | | 17% fresh) | | | |
| 26 | Test | 65:35 | 0.48 | No | 0.48 | 80 | Yes (2 hrs) |
| | Enzyme II | | | | | | |
| 27 | Test | 65:35 | 0.48 | yes (26 + | 0.32 | 80 | Yes (2 hrs) |
| | Enzyme II | | | 33% fresh) | | | |
| 28 | Test | 65:35 | 0.48 | yes (26, 27, + | 0.27 | 80 | Yes (2 hrs) |
| | Enzyme II | | | 25% fresh) | | | |
| 29 | Test | 65:35 | 0.48 | yes (26, 27, | 0.24 | 80 | Yes (2 hrs) |
| | Enzyme II | | | 28, + 20% | | | |
| | | | | fresh) | | | |
| 30 | Test | 65:35 | 0.48 | yes (26, 27, | 0.23 | 80 | Yes (2 hrs) |
| | Enzyme II | | | 28, 29, + | | | |
| | | | | 17% fresh) | | | |
| 31 | Test | 65:35 | 0.48 | No | 0.48 | 80 | Yes (1.5 hrs) |
| | Enzyme II | | | | | | |
| 32 | Test | 65:35 | 0.48 | Yes (31 + | 0.32 | 80 | Yes (1.5 hrs) |
| | Enzyme II | | | 33% fresh) | | | |
| 33 | Test | 65:35 | 0.48 | Yes (31, 32 + | 0.27 | 80 | Yes (1.5 hrs) |
| | Enzyme II | | | 25% fresh) | | | |
| 34 | Test | 65:35 | 0.48 | Yes (31, 32, | 0.24 | 80 | Yes (1.5 hrs) |
| | Enzyme II | | | 33 + 20% | | | |
| | | | | fresh) | | | |
| 35 | Test | 65:35 | 0.48 | Yes (31, 32, | 0.23 | 80 | Yes (1.5 hrs) |
| | Enzyme II | | | 33, 34 + 17% | | | |
| | | | | fresh) | | | |
| 36 | Test | 65:35 | 0.48 | Yes (35) | 0.18 | 80 | Yes (2 hrs) |
| | Enzyme II | | | | | | |

### Example 2: Preparation of Test Enzyme I

The support used for the preparation of Test Enzyme I was HybCPG VBC (Fe³⁺), which was prepared in accordance with the procedure described in WO 2015/115993. Lipozyme® TL 100 L, containing Thermomyces lanuginosus lipase, TLL, was immobilized on HybCPG VBC (Fe³⁺) support according to the protocol below.

An enzyme solution was first prepared containing 40 vol % Lipozyme® TL 100 L with 3.84 g/L (20 mM) citric acid and 0.01 g/L (0.05 mM) calcium chloride hexahydrate. The citric acid and calcium chloride hexahydrate were added to the 40 vol % Lipozyme® TL 100 L solution in the form of pre-made stock solutions with concentrations of 250 mM for citric acid, pH adjusted to pH 7, and 200 mM for the calcium chloride. The pH of the final TLL enzyme solution used for immobilization was 6.6. 1 L of enzyme solution was used with 25 g of HybCPG VBC support

The HybCPG VBC support was mixed with the TLL enzyme solution on a roller mixer for 1.5 hours at 60 rpm, whereafter the immobilized catalyst (Lipozyme® TL 100 L on HybCPG VBC) was left to sediment and the supernatant decanted. The immobilized catalyst was washed 3 times with a buffered solution (160 ml per 25 g of support) containing 5.78 g/L (75 mM) ammonium acetate, 23.8 g/L (100 mM) 4-(2-hydroxyethyl)-1-piperazineethanesulfonic acid (HEPES) and 2.19 g/L (10 mM) calcium chloride hexahydrate, pH 7. For each washing step, the immobilized catalyst was mixed with the washing solution for 1 minute on a roller mixer at 60 rpm, left to sediment and thereafter the supernatant was removed by decantation. The washing solution (70 ml per 25 g support) was then used to transfer the immobilized catalyst to a borosilicate glass 3.3 filter funnel (porosity grade 3) and left to dry for 1.5 hours by removing the liquid with the aid of a vacuum pump (150 mbar below atmospheric pressure). The support was then transferred to a crystallizing dish and subjected to a vacuum, (980 mbar below atmospheric pressure) for 48 hours.

The amount of immobilized TLL on EziG was determined by comparing the activity of the TLL enzyme solution before and after immobilization in an activity assay based on hydrolysis of tributyrin using a pH-stat. The reaction mixture was an emulsion of 5 ml tributyrin with 45 ml buffer (2.09 g/l, 10 mM, 3-morpholinopropane-1-sulfonic acid, 43.8 g/l, 0.2 M, calcium chloride hexahydrate and 20 g/l gum arabic, pH 7.5) and. The activities were determined at 40° C. and pH 7.5 for 4 min by measuring the amount of base solution (20 g/l, 0.5 M, sodium hydroxide) added per minute and volume of TLL enzyme solution. The activity of TLL in solution phase has previously been determined to be 5600 μmol per minute per mg of enzyme at 25° C. (M. Martinelle, M. Holmquist, K. Hult, Biochimica et Biophysica Acta (BBA)—Lipids and Lipid Metabolism, 1995, 1258, 272-276. https://doi.org/10.1016/0005-2760(95)00131-U) and was used to quantify the amount of immobilized TLL on the support. Using the protocol described herein the amount of TLL immobilized on the support was 0.08 g per gram of support. However, depending on the specifics of the preparation, the amount of TLL immobilized on the support could be adjusted from 0.08 g to 0.16 g per gram of support.

Test Enzyme I used in trials 6, 7, 8, 13, and 15 had 15.9% w/w bonded enzyme (enzyme loading). Test Enzyme I used in trials 16-25 contained 8.6% w/w bonded enzyme (enzyme loading).

### Example 3: Preparation of Test Enzyme II

The silica support used for the preparation was Cariact Q30 purchased from Fuji Silysia Chemical (Cariact Q30 70-150 μm particle size, 30 nm pore diameter, 99 m² surface area). The silica support (5 g) was transferred to a jacketed glass reactor with overhead stirrer and toluene was added (100 mL). The silica support surface was amino-functionalized using 15 mL (3-aminopropyl)trimethoxysilane, which was added to the reactor and incubated for 22 hours (250-290 rpm, 80° C.). The resultant solid was filtered under vacuum, washed with toluene (2×200 mL) and then washed with EtOH 99.7% (2×200 mL). After the washing steps, the solid was filtered using a glass filter funnel under vacuum for 60 min. The amino-functionalized support was transferred to a crystallizing dish and dried under vacuum overnight at 120° C.

The solution of Lipozyme® TL 100 L, containing Thermomyces lanuginosus lipase (TLL) was prepared for immobilization by dilution of the commercial enzyme solution (60% v/v) in deionised water (pH 6.7 of final solution). The diluted enzyme solution (250 mL) was transferred to a 500 mL Duran bottle containing 50 g of support (CPS Q30 functionalized with (3-aminopropyl)trimethoxysilane)). Samples were incubated on a roller mixer for 1.5 hours (70 rpm, ambient temperature). After immobilization the supernatant was removed and the supports were washed with a solution containing 50 mM HEPES, 50 mM ammonium acetate, 10 mM CaCl₂), pH 7 solution (1×250 mL), followed by a further wash with a solution containing 50 mM HEPES, 50 mM ammonium acetate, 10 mM CaCl₂), 130 mM sucrose, pH 7 solution (1×250 mL). For each washing step, the immobilized catalyst was mixed with the washing solution on a roller mixer for 3 min (70 rpm, ambient temperature). The catalyst was filtered on a borosilicate glass 3.3 filter funnel under vacuum to remove most of the washing solution (6 min), transferred to a 1 L glass flask and dried on a rotary evaporator for 115-120 minutes (40 mbar, 40° C.).

The amount of TLL immobilized on the support was typically 0.05 g per gram of support (5% enzyme loading), determined as described above for Test Enzyme I.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A process for batch enzymatic treatment of multiple lipid-containing compositions, the process comprising:
providing a lipid-containing composition;
contacting the lipid-containing composition with an immobilized enzyme material comprising a carrier and at least one enzyme immobilized on the carrier, wherein the amount of the immobilized enzyme material is about 0.72% or less (by weight of the lipid-containing composition);
mixing the lipid-containing composition with the immobilized enzyme material to form a first enzymatically treated composition, wherein the mixing occurs for about 10 hours or less;
separating the immobilized enzyme material from the first enzymatically treated composition;
contacting the immobilized enzyme material with at least one additional lipid-containing composition; and
mixing the at least one additional lipid-containing composition with the immobilized enzyme material to form a second enzymatically treated composition.

2. The process of claim 1, further comprising separating the immobilized enzyme material from the second enzymatically treated composition.

3. The process of claim 1, wherein the amount of the immobilized enzyme material is from about 0.15% to about 0.72% (by weight of the lipid-containing composition).

4. The process of claim 3, wherein the amount of the immobilized enzyme material is from about 0.18% to about 0.72% (by weight of the lipid-containing composition).

5. The process of claim 4, wherein the amount of the immobilized enzyme material is from about 0.18% to about 0.5% (by weight of the lipid-containing composition).

6. The process of claim 1, wherein the mixing occurs for from about 1.5 hours to about 10 hours.

7. The process of claim 1, wherein the lipid-containing composition is heated to a temperature of from about 70 to about 95° C.

8. The process of claim 1, wherein the enzymatic treatment is selected from the group consisting of interesterification, intraesterification, alcoholysis, acidolysis, glycerolysis, transesterification, and combinations thereof.

9. The process of claim 1, wherein the enzyme is derived from Achromobacter, Alcaligenes, Aspergillus, Bacillus, Burkholderia, Candida, Chromobacterium, Corynebacterium, Fusarium, Geotrichum, Humicolo, Humicora, Mucor, Penicillium, Pseudomonas, Rhizomucor, Rhizopus, Staphylococcus, Thermomyces, or Torulopsis.

10. The process of claim 9, wherein the enzyme is selected from the group consisting of Aspergillus niger Mucor mihei, Mucor javanicus Pseudomonas fluorescens, Rhizopus delemar, Burkholderia cepacia Candida cylindracea, Candida Antarctica Candida rogosa Fusarium solani Penicillium cyclopium, Rhizomucor miehei and Thermomyces lanuginosus.

11. The process of claim 10, wherein the enzyme is Thermomyces lanuginosus.

12. The process of claim 1, wherein the lipid-containing composition comprises an oil or fat selected from the group consisting of canola oil, castor oil, coconut oil, coriander oil, corn oil, cottonseed oil, hazelnut oil, hempseed oil, linseed oil, meadowfoam oil, olive oil, palm oil, palm kernel oil, peanut oil, rapeseed oil, rice bran oil, safflower oil, sasanqua oil, soybean oil, sunflower seed oil, tall oil, tsubaki oil, varieties of “natural” oils having altered fatty acid compositions via Genetically Modified Organisms (GMO) or traditional “breeding” such as high oleic or low linolenic, low saturated oils (high oleic canola oil, low linolenic soybean oil or high stearic sunflower oils), vegetable oil, menhaden, candlefish oil, cod-liver oil, orange roughly oil, sardine oil, herring oils, lard, tallow, algae oil, fish oil, animal-derived fat, waste cooking oil, brown grease, oil triglycerides derived from inedible plant sources, partial glycerides and free fatty acids derived from those oils, and combinations or blends thereof.

13. The process of claim 1, wherein the enzymatic treatment is carried out in a stirred-tank reactor operating in batch mode.
